(19) United States
(12) Patent Application Publication
(10) Pub. No.: US 2024/0284006 A1
(43) Pub. Date: Aug. 22, 2024

(54) DYNAMIC RECOMMENDATION ENGINE

(71) Applicant: Q Bio, Inc., San Carlos, CA (US)

(72) Inventors: Jeffrey Howard Kaditz, Wilson, WY (US); Robert Gardner Ostrow, San Francisco, CA (US)

(21) Appl. No.: 18/584,332

(22) Filed: Feb. 22, 2024

Related U.S. Application Data
(60) Provisional application No. 63/486,321, filed on Feb. 22, 2023.

(51) Int. Cl. H04N 21/466; G16H 30/00

(52) U.S. Cl. CPC H04N 21/4668; G16H 30/00

(57) ABSTRACT

A computer system that dynamically determines related content is described. During operation, the computer system may receive information (such as user-interface activity information) specifying selected content. In response, the computer system may dynamically determine the related content based at least in part on one or more prior expert selections that are associated with the selected content and/or one or more predefined or predetermined relationships with the selected content. Then, the computer system may provide the selected content and the determined related content.

### CROSS-REFERENCE TO RELATED APPLICATION

This application claims priority under 35 U.S.C. § 119(e) to: U.S. Provisional Application Ser. No. 63/486,321, entitled “Dynamic Recommendation Engine,” by Robert Gardner Ostrow and Jeffrey Howard Kaditz, Attorney Docket Number TSLH-P24.00-P, filed on Feb. 22, 2023 the contents of which are herein incorporated by reference.

### FIELD

The described embodiments relate generally to techniques for dynamically determining related content based at least in part on prior one or more expert selections that are associated with selected content and/or one or more predefined or predetermined relationships with the selected content.

### BACKGROUND

Many non-invasive characterization techniques are available for determining one or more physical parameters of a sample. For example, magnetic properties can be studied using magnetic resonance or MR (which is often referred to as ‘nuclear magnetic resonance’ or NMR), a physical phenomenon in which nuclei in a magnetic field absorb and re-emit electromagnetic radiation. Moreover, density variations and short or long-range periodic structures in solid or rigid materials can be studied using characterization techniques such as x-ray imaging, x-ray diffraction, computed tomography, neutron diffraction or electron microscopy, in which electromagnetic waves or energetic particles having small de Broglie wavelengths are absorbed or scattered by the sample. Furthermore, density variations and motion in soft materials or fluids can be studied using ultrasound imaging, in which ultrasonic waves are transmitted and reflected in the sample.

In each of these and other non-invasive characterization techniques, one or more external excitation (such as a flux of particles or incident radiation, static or time-varying scalar fields, and/or static or time-varying vector fields) are applied to the sample, and a resulting response of the sample, in the form a physical phenomenon, is measured to, directly or indirectly, determine the one or more physical parameters. For example, in MR magnetic nuclear spins may be partially aligned (or polarized) in an applied external DC magnetic field. These nuclear spins may precess or rotate around the direction of the external magnetic field at an angular frequency (which is sometimes referred to as the ‘Larmor frequency’) given by the product of a gyromagnetic ratio of a type of nuclei and the magnitude or strength of the external magnetic field. By applying a perturbation to the polarized nuclear spins, such as one or more radio-frequency (RF) pulses (and, more generally, electro-magnetic pulses) having pulse widths corresponding to the angular frequency and at a right-angle or perpendicular to the direction of the external magnetic field, the polarization of the nuclear spins can be transiently changed. The resulting dynamic response of the nuclear spins (such as the time-varying total magnetization) can provide information about the physical and material properties of a sample, such as one or more physical parameters associated with the sample.

Moreover, in general each of the characterization techniques may allow one or more physical parameters to be determined in small volumes or voxels in a sample, which can be represented using a tensor. Using magnetic resonance imaging (MRI) as an example, the dependence of the angular frequency of precession of nuclear spins (such as protons or the isotope 1H) on the magnitude of the external magnetic field can be used to determine images of three-dimensional (3D) or anatomical structure and/or the chemical composition of different materials or types of tissue. Notably, by applying a non-uniform or spatially varying magnetic field to a sample, the resulting variation in the angular frequency of precession of ¹H spins is typically used to spatially localize the measured dynamic response of the ¹H spins to small volumes or voxels, which can be used to generate images, such as of the internal anatomy of a patient.

However, it can be difficult to concisely present measurement data in a meaningful and intuitive manner. For example, non-invasive characterizations techniques typically collect a significant amount of data. Consequently, there is often too much data to present to a user in a user interface or at a given time. Instead, the user usually uses the user interface to navigate through the data over time, such as by scrolling through a sequence of images that are sequentially displayed. This problem may be compounded as additional characterization techniques are performed on the same subject. The difficulty in viewing and understanding the ever-increasing amount of available data is frustrating to healthcare providers, and increases the risk of errors that can adversely impact patient heath and outcomes.

### SUMMARY

A computer system that dynamically determines related content is described. This computer system includes: a processor that executes program instructions; and memory that stores the program instructions. During operation, the computer system receives information (such as user-interface activity information) specifying selected content. In response, the computer system dynamically determines the related content based at least in part on one or more prior expert selections that are associated with the selected content and/or one or more predefined or predetermined relationships with the selected content. Then, the computer system provides the selected content and the determined related content.

For example, the selected content may include an image associated with a non-invasive characterization technique. Notably, the non-invasive characterization technique may include: MRI, computed tomography, x-ray imaging or ultrasound. In some embodiments, the image may correspond to a portion of a human body.

Moreover, the selected content may include or may be associated with a biomarker, and the one or more predefined or predetermined relationships may be between the biomarker and one or more additional biomarkers.

Furthermore, the one or more predefined or predetermined relationships may be based at least in part on: genetics information, chemistry, a behavior, an environmental factor, a disease, a comorbidity and/or a biological pathway.

Additionally, the one or more predefined or predetermined relationships may be based at least in part on medical literature.

In some embodiments, the related content may include a measurement associated with a different measurement technique than the non-invasive characterization technique.

Moreover, the content may be associated with a patient, and the one or more prior expert selections may be by a physician and may be associated with one or more different patients from the patient. Note that the patient and the one or more different patients may share one or more characteristics or attributes. For example, the patient and the one or more different patients may have a disease (such as the patient and the one or more different patients may have been diagnosed as having the disease), a risk of having the disease, and/or may be members of a demographic group or a common population.

Furthermore, providing the selected content and the determined related content may include displaying the selected content and the determined related content on a display.

Additionally, providing the determined related content may include providing a user interface with user-interface features (such as one or more virtual icons) corresponding to the determined related content.

Another embodiment provides a computer-readable storage medium for use with the computer system. This computer-readable storage medium includes program instructions that, when executed by the computer system, causes the computer system to perform at least some of the aforementioned operations.

Another embodiment provides a method for performing automatic set up. This method includes at least some of the aforementioned operations performed by the computer system.

This Summary is provided for purposes of illustrating some exemplary embodiments, so as to provide a basic understanding of some aspects of the subject matter described herein. Accordingly, it will be appreciated that the above-described features are simply examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a block diagram illustrating an example of a system in accordance with an embodiment of the present disclosure.

FIG. 2 is a flow diagram illustrating an example of a method for determining model parameters associated with a sample in accordance with an embodiment of the present disclosure.

FIG. 3 is a drawing illustrating an example of communication among components in the system in FIG. 1 in accordance with an embodiment of the present disclosure.

FIG. 4 is a drawing illustrating an example of a machine-learning model in accordance with an embodiment of the present disclosure.

FIG. 5 is a drawing illustrating an example of a neural model in accordance with an embodiment of the present disclosure.

FIG. 6 is a drawing illustrating an example of classification or segmentation of one or more anatomical structures in the sample in accordance with an embodiment of the present disclosure.

FIG. 7 is a flow diagram illustrating an example of a method for determining coefficients in a representation of coil sensitivities and MR information associated with a sample in accordance with an embodiment of the present disclosure.

FIG. 8 is a drawing illustrating an example of communication among components in the system in FIG. 1 in accordance with an embodiment of the present disclosure.

FIG. 9 is a flow diagram illustrating an example of a method for dynamically determining related content in accordance with an embodiment of the present disclosure.

FIG. 10 is a drawing illustrating an example of communication among components in the system in FIG. 1 in accordance with an embodiment of the present disclosure.

FIGS. 11-46 are drawings illustrating examples of user interfaces in accordance with an embodiment of the present disclosure.

FIG. 47 is a drawing illustrating an example of a protocol diagnostic tool in accordance with an embodiment of the present disclosure.

FIG. 48 is a drawing illustrating an example of a graph relating a demographic with a pathology in accordance with an embodiment of the present disclosure.

FIG. 49 is a drawing illustrating an example of a graph relating a pathology to a biomarker in accordance with an embodiment of the present disclosure.

FIG. 50 is a drawing illustrating an example of a graph relating a pathology to a biomarker in accordance with an embodiment of the present disclosure.

FIG. 51 is a drawing illustrating an example of an evidence relationship in accordance with an embodiment of the present disclosure.

FIG. 52 is a block diagram illustrating an example of an electronic device in accordance with an embodiment of the present disclosure.

FIG. 53 is a drawing illustrating an example of a data structure that is used by the electronic device of FIG. 52 in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

A computer system that dynamically determines related content is described. During operation, the computer system may receive information (such as user-interface activity information) specifying selected content. In response, the computer system may dynamically determine the related content based at least in part on one or more prior expert selections that are associated with the selected content and/or one or more predefined or predetermined relationships with the selected content. Then, the computer system may provide the selected content and the determined related content.

By dynamically determining the related content, the disclosed computing techniques may enable efficient navigation, viewing or use of large datasets. Notably, the computing techniques may allow useful related content to be presented to a user (such as a physician or a healthcare provider) along with selected content, thereby allowing the user to quickly access useful information that is associated with the selected content. This related content may improve monitoring of an individual's health, diagnosing of disorders or diseases, selecting treatment(s) and/or assessing outcomes of the treatment(s). In the process, the computing techniques may allow the user to use related portions of large datasets to improve patient health. Consequently, the computing techniques may improve the user experience when using the computer system.

While MR measurements are used as an illustration in the present discussion, in general the set-up techniques may be used in conjunction with a variety of characterization techniques and/or forward models that quantitatively simulate the response physics occurring within the sample to a given excitation. For example, the characterization techniques may involve: x-ray measurements (such as x-ray imaging, x-ray diffraction or computed tomography), neutron measurements (neutron diffraction), electron measurements (such as electron microscopy or electron spin resonance), optical measurements (such as optical imaging or optical spectroscopy that determines a complex index of refraction at one or more wavelengths), infrared measurements (such as infrared imaging or infrared spectroscopy that determines a complex index of refraction at one or more wavelengths), ultrasound measurements (such as ultrasound imaging), proton measurements (such as proton scattering), MR measurements or an MR technique (such as MRI, MR spectroscopy or MRS with one or more types of nuclei, magnetic resonance spectral imaging or MRSI, MR elastography or MRE, MR thermometry or MRT, magnetic-field relaxometry, diffusion-tensor imaging and/or another MR technique, e.g., functional MRI, metabolic imaging, molecular imaging, blood-flow imaging, etc.), impedance measurements (such as electrical impedance at DC and/or an AC frequency) and/or susceptibility measurements (such as magnetic susceptibility at DC and/or an AC frequency). Therefore, the excitation may include at least one of: an electromagnetic beam in an x-ray band of wavelengths (such as between 0.01 and 10 nm), a neutron beam, an electron beam, an electromagnetic beam in an optical band of wavelengths (such as between 300 and 800 nm), an electromagnetic beam in an infrared band of wavelengths (such as between 700 nm and 1 mm), a sound wave in an ultrasound band of wavelengths (such as between 0.2 and 1.9 mm), a proton beam, an electric field associated with an impedance measurement device, a radio-frequency wave associated with an MR apparatus or scanner, and/or a magnetic field associated with a susceptibility measurement device. However, another non-invasive characterization technique (such as positron emission spectroscopy), an integrated therapy (such as proton beam therapy or proton implantation, radiation therapy, magnetically guided nano particles, etc.) and/or a different range of wavelengths (such as ultraviolet wavelengths between 10 and 400 nm) may be used. In general, the set-up techniques may be used with a wide variety of excitation may be used to ‘excite’ a region of space as long as there is a forward model that describes the response physics for these excitations. In the discussion that follows, an MR technique is used as an illustrative example of a characterization technique.

Note that the sample may include an organic material or an inorganic material. For example, the sample may include: an inanimate (i.e., non-biological) sample, a biological lifeform (such as a person or an animal, i.e., an in-vivo sample), or a tissue sample from an animal or a person (i.e., a portion of the animal or the person). In some embodiments, the tissue sample was previously removed from the animal or the person. Therefore, the tissue sample may be a pathology sample (such as a biopsy sample), which may be formalin fixed-paraffin embedded. In the discussion that follows, the sample is a person or an individual, which is used as an illustrative example.

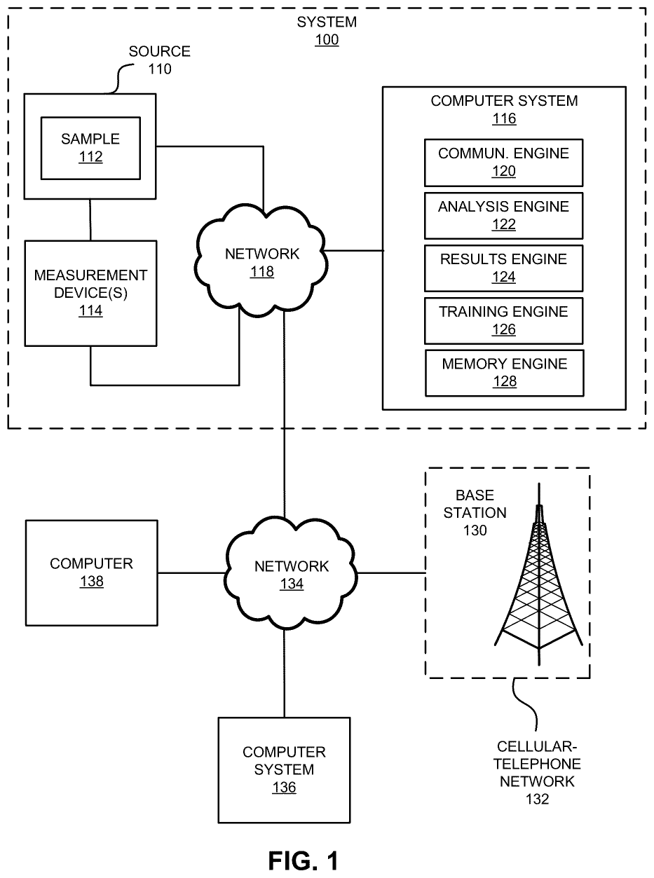

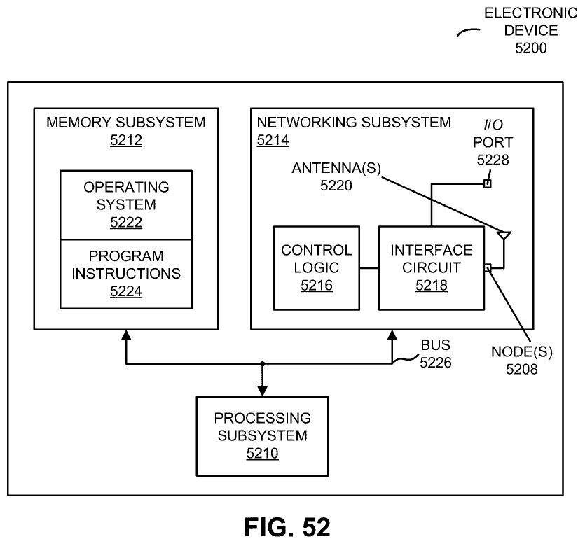

We now describe embodiments of a system. FIG. 1 presents a block diagram illustrating an example of a system 100. In system 100, a source 110 selectively provides an excitation to a sample 112, and a measurement device 114 selectively performs measurements on sample 112 to measure a response of sample 112 to the excitation. Moreover, system 100 includes a computer system 116, which may include one or more computers. As described further below with reference to FIG. 52, computer system 116 may include subsystems, such as a processing subsystem, a memory subsystem and a networking subsystem. For example, the processing subsystem may include a processor that executes program instructions, the memory subsystem may include a memory that stores the program instructions, and networking subsystem may include an interface that communicates instructions or commands to source 110 and measurement device 114 (such as one or more sensors), that receives measurements from measurement device 114, and that selectively provides determined model parameters.

During operation, a communication engine (or module) 120 in computer system 116 may provide, via an optional network 118 (such as one or more wired and/or wireless links or interconnects), an instruction or a command to source 110, which may cause source 110 to apply, to sample 112, the excitation. This excitation may have at least a wavelength and an intensity or a flux. For example, the excitation may include: electromagnetic radiation, a radio-frequency wave, a particle beam, a sound wave, a magnetic field, and/or an electric field.

In some embodiments, the excitation may include an external magnetic field that polarizes one or more types of nuclei in sample 112, an optional gradient in the magnetic field, and/or an RF pulse sequence (which are sometimes referred to as ‘measurement conditions’ or ‘scanning instructions’). Thus, source 110 may include a magnet that applies the external magnetic field, an optional gradient coil that applies the optional gradient, and/or an RF coil that applies the RF pulse sequence.

Then, communication engine 120 may provide, via network 118, an instruction or a command to measurement device 114, which may cause measurement device 114 to perform measurements of the response of at least a portion of sample 112 to the excitation. Moreover, measurement device 114 may provide, via network 118, the measurement results to communication engine 120. Note that measurement device 114 may include: an x-ray detector, a neutron detector, an electron detector, an optical detector, an infrared detector, an ultrasound detector, a proton detector, an MR apparatus or scanner, the impedance measurement device (such as a gel-covered table in an MR apparatus or scanner) and/or the susceptibility measurement device.

In some embodiments, measurement device 114 may include one or more RF pickup coils or another magnetic sensor (such as a magnetometer, a superconducting quantum interference device, opto-electronics, etc.) that measure time-varying or time-domain electrical signals corresponding to the dynamic behavior of nuclear spins in the one or more types of nuclei or at least an average component of the magnetization corresponding to the aggregate dynamic behavior of the nuclear spins (which is sometimes referred to as a ‘magnetic response’) of at least the portion of sample 112. For example, measurement device 114 may measure the transverse magnetization of at least a portion of sample 112 as it precesses in the xy plane.

Note that the measurements provided by measurement device 114 may be other than or different from an image. For example, the measurements may be other than MRI results. For example, the measurements may include or may correspond to (such as one or more components of) a free-induction-decay of the nuclear spins in sample 112. Consequently, in some embodiments the measurements may not involve performing a Fourier transform on the measured electrical signals (and, thus, may not be performed in k-space and may not involve pattern matching in k-space, such as MR fingerprinting). However, in general, the measurements may be specified in the time domain and/or the frequency domain. Therefore, in some embodiments, a variety of signal processing (such as filtering, image processing, etc.), noise cancellation and transformation techniques (such as a discrete Fourier transform, a Z transform, a discrete cosine transform, data compression, etc.) may be performed on the measurements.

After receiving the measurements, analysis engine (or module) 122 in computer system 116 may analyze the measurements. This analysis may involve determining a (possibly time-varying) 3D position of sample 112 relative to measurement device 114 (which is sometimes referred to as ‘3D registration information’). For example, the aligning may involve performing point-set registration, such as with reference markers at known spatial locations. The registration may use a global or a local positioning system to determine changes in the position of sample 112 relative to measurement device 114. Alternatively or additionally, the registration may be based at least in part on variation in the Larmor frequency and the predetermined spatial magnetic-field inhomogeneity or variation in the magnetic field of source 110 and/or measurement device 114 (such as an MR apparatus or scanner). In some embodiments, the analysis involves aligning the voxels based at least in part on the registration information with desired voxel locations, and/or resampling and/or interpolating the measured signals to different voxel locations, which may facilitate subsequent comparisons with previous measurements or results.

Moreover, analysis engine 122 may use the measurements to determine model parameters for a forward model with multiple voxels that represent sample 112, and that simulates the response physics occurring in sample 112 to a given excitation in a range of possible excitations (i.e., the forward model may be more general than one that determines the predicted response to a particular or a specific excitation). Notably, with the appropriate model parameters for the voxels in sample 112, analysis engine 122 may use the forward model to accurately and quantitatively simulate or calculate a predicted response of sample 112 to the excitation (such as a predicted component of the magnetization). Note that the forward model may be based at least in part on or may use one or more differential equations or one or more phenomenological equations that approximates the response physics of sample 112 on a voxel-by-voxel basis. For example, the forward model may be based at least in part on or may use the Bloch equations, the Bloch-Torrey equations (thus, the forward model may include simulations of dynamics, such as motion associated with: respiration, a heartbeat, blood flow, mechanical motion, etc.), full Liouvillian computations (such as a Liouville supermatrix of interactions between two or more elements), a full Hamiltonian, Maxwell's equations (e.g., the forward model may calculate magnetic and electrical properties of sample 112), thermal diffusion equations, the Pennes equations, and/or another simulation technique that represents the physics of a response of sample 112 to a type of excitation. Because in some embodiments the assumptions underlying the Bloch equations are invalid (such as the parallel and antiparallel components of the magnetization are coupled, e.g., when the state of the magnetization is not reset prior to an RF pulse sequence), additional error terms may be added to the Bloch equations. Therefore, the forward model may be able to compute a dynamic (e.g., time-varying) state of sample 112 in response to an arbitrary excitation in a range of possible excitations or excitation values.

In some analysis approaches, computer system 116 may determine the model parameters by solving an inverse problem by iteratively modifying the model parameters associated with the voxels in the forward model until a difference between the predicted response and the measured dynamic magnetic response is less than a predefined value (such as 0.1, 1, 5 or 10%). (Note that ‘an inverse problem’ starts with one or more result(s) or output(s) and then calculates the inputs or causes. This is the inverse of a ‘forward problem,’ which starts with the inputs and then calculates the one or more results or the outputs.) However, in this ‘iterative approach,’ source 110 may repeatedly apply different excitations, and measurement device 114 may repeatedly perform corresponding measurements. Consequently, the iterative approach may be time-consuming, expensive and complicated. Thus, the iterative approach may consume significant resources in system 100 until the appropriate model parameters are determined.

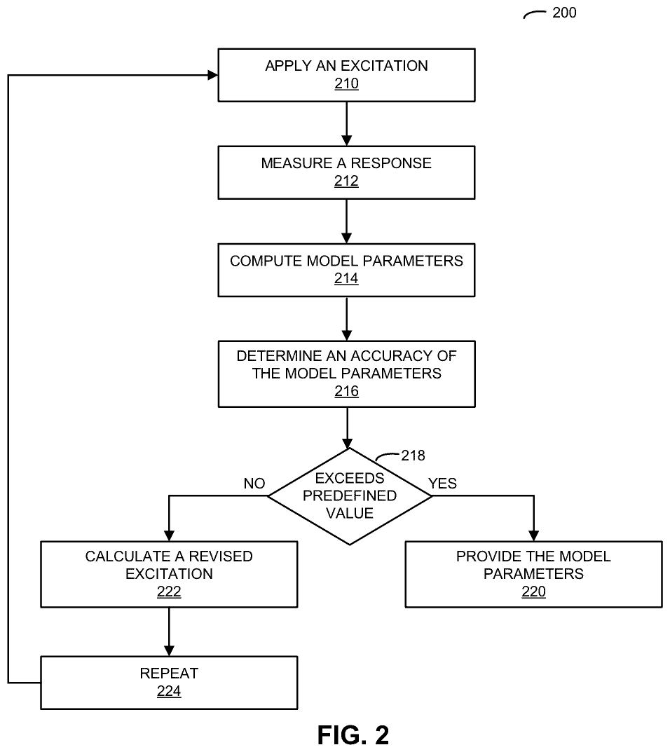

As described further below with reference to FIGS. 2-5, in order to address these problems, in disclosed computation techniques analysis engine 122 may use one or more predetermined or pretrained predictive models (such as a machine-learning model or a neural network, which may be specific to a particular sample or an individual, e.g., the predictive model may be a personalized predictive model) to, at least in part, compute the model parameters on a voxel-by-voxel basis. For example, analysis engine 122 may use the measurements and information specifying the excitation as inputs to a predictive model, which provides, as an output, the model parameters associated with the voxels. Therefore, the predictive model may be trained on or may incorporate model-parameter information based at least in part on measurements or measurement results. In some embodiments, the predictive model may correct the measurements for extrinsic characteristics or a signature of a specific source 110 and/or measurement device 114 (such as RF noise or spatial magnetic-field inhomogeneity) and/or a particular excitation or measurement condition, so that the determined model parameters are intrinsic to sample 112 at a particular time when the measurements were performed.

Note that the model parameters may include: a spin-lattice relaxation time T₁ (which is the time constant associated with the loss of signal intensity as components of the nuclear-spin magnetization vector of a type of nuclei relax to be parallel with the direction of an external magnetic field), a spin-spin relaxation time T₂ (which is the time constant associated with broadening of the signal during relaxation of components of the nuclear-spin magnetization vector of a type of nuclei perpendicular to the direction of the external magnetic field), an adjusted spin-spin relaxation time T_{2*}, proton or nuclei density (and, more generally, the densities of one or more type of nuclei), diffusion (such as components in a diffusion tensor), velocity/flow, temperature, off-resonance frequency, electrical conductivity or a dielectric constant, and/or a magnetic susceptibility or permittivity.

If a subsequent simulation using these model parameters provided by the predictive model, the forward model and one or more excitations of one or more predicted responses of sample 112 (such as a simulated or predicted MR signal) agrees with the corresponding measurements (such as a difference between a predicted response and a measurement is less than a predefined value, e.g., 0.1, 1, 5 or 10%, or alternatively when an accuracy exceeds a predefined value), results engine (or module) 124 in computer system 116 may provide the determined model parameters, such as by providing an output to a user, to another electronic device, to a display and/or to the memory. In some embodiments, results engine 124 may output a tensor field map for sample 112 with model parameters for 3 spatial x one temporal x up to N measurement dimensions, where each measurement may be a vector or scalar quantity.

Thus, when the accuracy exceeds the predefined value (such as 90, 95, 99 or 99.9%), the model parameters may be computed in a single pass without further iteration. Consequently, the model parameters having an accuracy exceeding the predefined value may be computed using fewer (or no) iterations with the predetermined predictive model (and, thus, more rapidly) than in the iterative approach without the predetermined predictive model.

Alternatively, when the accuracy is less than the predefined value, computer system 116 may perform one or more iterations in which one or more different, modified or revised excitations (such as a different RF pulse sequence) are applied to sample 112 by source 114, and one or more corresponding additional measurements are performed by measurement device 114. These one or more additional measurements may be used by computer system 116 to determine the model parameters with an accuracy less than the predefined value.

For example, analysis engine 122 may use a second predetermined predictive model (such as a second machine-learning model or a second neural network) to determine a revised excitation. Notably, using information specifying the excitation and the accuracy as inputs, the second predictive model may output the revised excitation. Then, system 100 may repeat the applying, measuring, computing and determining operations with the revised excitation instead of the excitation. Therefore, the second predictive model may be trained on or may incorporate excitation information based at least in part on remaining differences between the predicted response and the measurement in order to reduce or eliminate the remaining differences in one or more subsequent iterations of the operations performed by system 100. In some embodiments, the second predictive model may revise a sampling frequency, a characterization technique, etc. to determine additional information that allows the determination of the model parameters using the first predictive model to converge (i.e., to have an accuracy less than the predefined value). Stated differently, the next perturbation or disturbance may be chosen to minimize the error or the difference across the hyper-dimensional space.

In some embodiments, when the accuracy is less than the predefined value, training engine (or module) 126 in computer system 116 may: add the excitation and the measured response to a training dataset; and determine, using the training dataset, a revised instance of the predictive model for subsequent use in determining the model parameters. Thus, the measurements performed by system 100 may be selectively used in an adaptive learning technique to improve the predictive model and, therefore, the determined model parameters for a range of excitations (such as different values of the wavelength and the intensity or the flux).

Using the model parameters and the forward model, analysis engine 122 may simulate or predict a response of sample 112 to an arbitrary excitation, such as an arbitrary external magnetic field strength or direction (such as 0 T, 6.5 mT, 1.5 T, 3 T, 4.7 T, 9.4 T, and/or 15 T, or a time-varying direction, e.g., a slowly rotating external magnetic field), an arbitrary optional gradient, an arbitrary RF pulse sequence, an arbitrary magnetic state or condition (e.g., in which the magnetization or polarization of sample 112 is not returned to, been reset to or re-magnetized to an initial state prior to a measurement), etc. Therefore, the model parameters and the forward model may be used to facilitate fast and more accurate measurements, such as: soft-tissue measurements, morphological studies, chemical-shift measurements, magnetization-transfer measurements, MRS, measurements of one or more types of nuclei, Overhauser measurements, and/or functional imaging. For example, in embodiments where computer system 116 determines the model parameters concurrently with measurements performed on sample 112 by source 110 and measurement device 114 (i.e., in real time), system 100 may rapidly characterize one or more physical parameters of sample 112 (at the voxel level or on average) on time scales smaller than T₁ or T₂ in an arbitrary type of tissue. This capability may allow system 100 to perform initial measurements to determine the model parameters, and then to use the determined model parameters to simulate or predict MR signals to complete or fill in ongoing measurements being performed by system 100, so that the results can be obtained more rapidly (and, thus, with a shorter MR scan time). Note that, in some embodiments, system 100 may determine the results (such as detecting an anomaly or a change in sample 112) based at least in part on quantitative comparisons of previous results obtained on sample 112, such as stored model parameters for the voxels in sample 112 that were determined during a previous MR scan(s) of sample 112. Such comparisons may be facilitated by 3D registration information that allows the voxel positions in sample 112 at different times to be aligned. In some embodiments, the results are based at least in part on a physician's instructions, medical lab test results (e.g., a blood test, urine-sample testing, biopsies, genetic or genomic testing, etc.), an individual's medical history, the individual's family history, quantitative tensor field maps with voxel-dependent multi-dimensional data for sample 112 or other samples, impedance of sample 112, a hydration level of sample 112 and/or other inputs.

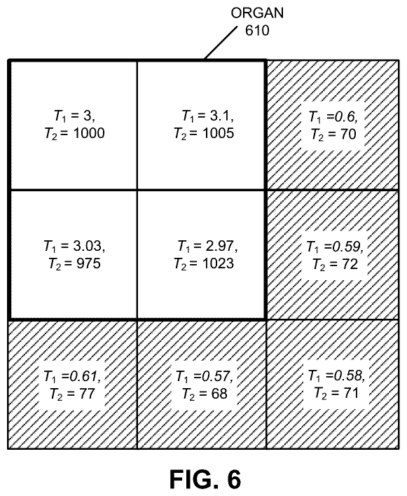

Furthermore, as described further below with reference to FIG. 6, in some embodiments analysis engine 122 may classify or segment one or more anatomical structures in sample 112 using the determined model parameters and a third predetermined predictive model (such as a third machine-learning model and/or a third neural network). For example, using the simulated or predicted response of sample 112 at the voxel level or the determined model parameters at the voxel level, the third predictive model may output the locations of different anatomical structures and/or may output classifications of different voxels (such as a type of organ, whether they are associated with a particular disease state, e.g., a type of cancer, a stage of cancer, etc.). Therefore, in some embodiments, the third predictive model may be trained on or may incorporate classification of segmentation information based at least in part on variation in the model parameters across boundaries between different voxels (such as discontinuous changes). This capability may allow analysis engine 122 to identify different anatomical structures (which may assist in the determination of the model parameters) and/or to diagnose or to make a diagnosis recommendation about a medical condition or a disease state. In some embodiments, the classification or segmentation is performed prior to, concurrently or following the determination of the model parameters.

In some embodiments, training engine 126 may have, at least in part, trained the predictive model, the second predictive model and/or the third predictive model using a simulated dataset. For example, training engine 126 may have generated the simulated dataset using the forward model, a range of model parameters and a range of excitations. In this way, simulated data may be used to accelerate training of one or more predictive models.

Notably, because the computation techniques may capture all relevant information during the measurements (such as an MR scan), the forward model can be used in an off-line mode to curate an extensive, labeled dataset that includes a large number of possible scenarios (such as different measurement conditions). This database can then be used to train predictive models. This capability may address the difficulty in obtaining MR data that is accurately labeled, reproducible, and artifact-free.

In conjunction with the generated dataset, one or more predictive models can be used to select regularization that accelerates the initial data acquisition and/or denoising. Moreover, the one or more predictive models can also be used to accelerate simulations or reconstruction using the forward model. For example, a predictive model can provide initial model parameters for use in the forward model, which may reduce the number of iterations required for the measurements and the simulations to converge on a solution that has an accuracy exceeding the predefined value. Thus, if the initial model parameters result in predicted response that are very different from the measurements, this can be feedback into the subsequent measurements and simulations to improve the model parameters and, thus, the predicted response.

Furthermore, if there is a portion of the model-parameter space that is not covered by the predictive model(s), new data points can be accurately generated and labeled to train the predictive model(s). Additionally, the predictive model(s) may be trained based on different metrics corresponding to different applications. For example, the predictive model(s) may be training to optimize the excitations used in difference scenarios (such as fast scanning for asymptomatic population, high accuracy for specific tissue properties, robustness to variations in the signal-to-noise ratio, different hardware imperfections, etc.).

In some embodiments, analysis engine 122 may run a neural network that determines first model parameters based at least in part on measured or simulated data and may performs brute-force nonlinear numerical calculations to solve an inverse problem using the measured or the simulated data to determine second model parameters. The difference between the first and the second model parameters from these two ‘inverse solvers’ may be used as the error in the neural-network-based approach. This approach may allow the neural network to learn because the numerical approach may be able to give real-time feedback to the neural network and to back propagate/update the weights in the neural network. This hybrid approach would still not require or need a priori training, but would be able to leverage the pattern-matching benefits of large neural networks with the determinism and accuracy of simulation/numerical techniques to solve the inverse problem. The hybrid approach may assist the neural network when it has an input unlike any of the examples used to train it. Similarly, the hybrid approach may be used to go directly from time-domain measurement to the model-parameterized output (i.e., the inverse problem outputs). In some embodiments, the hybrid approach is implemented using a generative adversarial network (GAN).

Note that, in some embodiments, the forward model may be independent of a particular MR apparatus or scanner. Instead, the forward model may be, e.g., specific to an individual. The predicted response computed using the forward model may be adjusted to include characteristics or a signature of a particular MR apparatus or scanner, such as magnetic-field inhomogeneity or spatial variation in the magnetic field, RF noise, a particular RF pickup coil or another magnetic sensor, variation in the characteristics or the signature with the external magnetic-field strength or the measurement conditions (such as the voxel size), geographic location, time (due to, e.g., magnetic storms), etc. Thus, the predicted response may be machine-specific.

While the preceding discussion illustrated the computation techniques using a single predictive model for sample 112, in other embodiments there may be multiple predictive models for sample 112. For example, different predictive models may be used to determine the model parameters for different portions of sample 112 (such as different organs or different types of tissue) and, thus, for different voxels. Therefore, in some embodiments different predictive models may be used to provide T₁ and T₂ values in different types of tissue, such as the values summarized in Table 1.

TABLE 1

| Tissue | T₁ (s) | T₂ (ms) |
|---|---|---|
| Cerebrospinal Fluid | 0.8-20 | 110-2000 |
| White Matter | 0.76-1.08 | 61-100 |
| Gray Matter | 1.09-2.15 | 61-109 |
| Meninges | 0.5-2.2 | 50-165 |
| Muscle | 0.95-1.82 | 20-67 |
| Adipose | 0.2-0.75 | 53-94 |

Additionally, in some embodiments, analysis engine 122 may receive information associated with or specifying results of measurements (which are sometimes referred to as non-invasive measurements) performed on an individual from measurement device 114 or may access or obtain the information in local or remote memory in or associated with system 100 via memory engine 128 (or module). Moreover, analysis engine 122 may access or obtain, via memory engine 128, historical non-invasive measurements (which were performed on the individual or a group of individuals, and which may include or may exclude the individual) and a dictionary of predetermined features or basis functions associated with the historical non-invasive measurements.

Note that the non-invasive measurements and the historical non-invasive measurements may include or correspond to MR measurements. For example, the MR measurements may include MR or MRI scans. Moreover, the non-invasive measurements and the historical non-invasive measurements may include MR parameters associated with voxels in the individual. Notably, the parameters may include: a density of a type of nuclei, a longitudinal relaxation time along a direction parallel to an external magnetic field and/or a transverse relaxation time along a direction perpendicular to the external magnetic field.

Then, analysis engine 128 may update the dictionary of predetermined features based at least in part on the non-invasive measurements and the historical non-invasive measurements. Notably, the updating may include performing a minimization technique with a cost or error function having an L2-norm term and an LO-norm term. Next, analysis engine 128 may determine (e.g., using a gradient-descent technique) weights associated with features in the updated dictionary of predetermined features (such as weights in a weighted linear superposition of the features in the updated dictionary) based at least in part on the non-invasive measurements. For example, the weights may be determined by performing a least-squares fit of the non-invasive measurements based at least in part on the features in the updated dictionary and/or by using the gradient-descent technique. In some embodiments, the weights may be determined using a pretrained machine-learning model (such as a supervised-learning model or a neural network) that outputs the weights based at least in part on the in the updated dictionary of predetermined features and the non-invasive measurements. In conjunction with the features in the updated dictionary, the weights may provide a sparse representation of the non-invasive measurements. Note that the dictionary of predetermined features and the updated dictionary of predetermined features may correspond to a portion of an anatomy of the individual.

Furthermore, results engine 124 may present or provide the sparse representation, such as by providing an output to a user, to another electronic device, to a display and/or to the memory (via memory engine 128).

In some embodiments, analysis engine 122 may perform analysis on the sparse representation of the non-invasive measurements. For example, the non-invasive measurement may include at least a component of a magnetization associated with the individual, and analysis engine 122 may: calculate at least a predicted component of the magnetization for the voxels associated with the individual based at least in part on the measured component of the magnetization, a forward model, the external magnetic field and an RF pulse sequence used to measure the non-invasive measurement; and solve an inverse problem by iteratively modifying the parameters associated with the voxels in the forward model until a difference between the predicted component of the magnetization and the measured component of the magnetization is less than a predefined value (such as a difference or error of 1, 5 or 10%).

Alternatively or additionally, analysis engine 122 may perform a longitudinal analysis of the non-invasive measurements based at least in part on the sparse representation and sparse representations of at least a subset of the historical non-invasive measurements. Note that the longitudinal analysis may not require or use a pixel-level registration operation between the non-invasive measurements and the subset of the historical non-invasive measurements. Moreover, by using the prior information, the longitudinal analysis may be used to detect changes, e.g., in an anatomical feature and, more generally, in the non-invasive measurements relative to a baseline provided by at least the subset of the historical non-invasive measurements. Thus, if it is known that an individual is at risk for liver cancer, more time may be spent acquiring high-quality images of their liver. Based at least in part on the detected changes, communication engine 120 may provide instructions to source 110 and/or measurement device 114 to perform an additional non-invasive measurement of at least a portion of the individual associated with the change. Results engine 124 may present or provide results of the longitudinal analysis (e.g., information specifying the detected change), such as by providing an output to the user, to another electronic device, to the display and/or to the memory (via memory engine 128).

While system 100 may include separate components (such as computer system 116, source 110 and measurement device 114) that communicate via network 118, in some embodiments some or all of these components may be integrated into a single device. Therefore, in some embodiments, computer system 116 may include source 110 and/or measurement device 114. In some embodiments, system 100 may be implemented using a single computer, thereby eliminating a need for network 118.

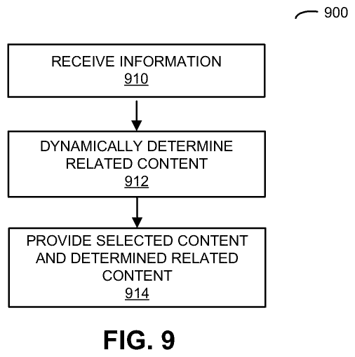

Moreover, as described further below with reference to FIGS. 9-46, in some embodiments computer system 116 or computer system 136 may perform the disclosed computing techniques. In the discussion that follows, computer system 136 is used to illustrate the computing techniques.

In some embodiments, electronic devices or components in FIG. 1 communicate packets in accordance with a wireless communication protocol, such as: a wireless communication protocol that is compatible with an IEEE 802.11 standard (which is sometimes referred to as ‘Wi-Fi®,’ from the Wi-Fi Alliance of Austin, Texas), Bluetooth or Bluetooth low energy (BLE), an IEEE 802.15.4 standard (which is sometimes referred to as Zigbee), a low-power wide-area network (LoRaWAN), a cellular-telephone network or data network communication protocol (such as a third generation or 3G communication protocol, a fourth generation or 4G communication protocol, e.g., Long Term Evolution or LTE or 5GC (from the 3rd Generation Partnership Project of Sophia Antipolis, Valbonne, France), LTE Advanced or LTE-A, another cellular-telephone data communication protocol, a fifth generation or 5G communication protocol, or other present or future developed advanced cellular communication protocol), and/or another type of wireless interface (such as another wireless-local-area-network interface). For example, an IEEE 802.11 standard may include one or more of: IEEE 802.11a, IEEE 802.11b, IEEE 802.11g, IEEE 802.11-2007, IEEE 802.11n, IEEE 802.11-2012, IEEE 802.11-2016, IEEE 802.11ac, IEEE 802.11ax, IEEE 802.11ba, IEEE 802.11be, or other present or future developed IEEE 802.11 technologies. Moreover, an access point, a radio node, a base station or a switch in the wireless network and/or the cellular-telephone network may communicate with a local or remotely located computer (such as a controller) using a wired communication protocol, such as a wired communication protocol that is compatible with an IEEE 802.3 standard (which is sometimes referred to as ‘Ethernet’), e.g., an Ethernet II standard. However, a wide variety of communication protocols may be used in the system, including wired and/or wireless communication. In the discussion that follows, Ethernet, Wi-Fi and/or cellular-telephone data communication protocol are used as illustrative examples.

In FIG. 1, computer system 116 may communicate with computer system 136 via network 118 and/or cellular-telephone network 132 (which may include a base station 130 and/or one or more radio nodes, which are not shown) and network 134 (such as a LAN, an intra-net or the Internet). Note that network 118 may include a WLAN, and communication in the WLAN may occur via one or more access points (not shown). In some embodiments, an access points may include a physical access point and/or a virtual access point that is implemented in software in an environment of an electronic device or a computer, such as computer system 116. Alternatively or additionally, computer system 116 may perform the functions of an access point. Moreover, the one or more radio nodes 118 may include: an Evolved Node B (eNodeB), a Universal Mobile Telecommunications System (UMTS) NodeB and radio network controller (RNC), a New Radio (NR) gNB or gNodeB (which communicates with a network with a cellular-telephone communication protocol that is other than LTE), etc. Thus, computer system 116 and computer system 136 may communicate with each other using a wired communication protocol and/or a wireless communication protocol. Note that networks 118 and 134 may be the same or different networks. Moreover, in the discussion that follows, an access point, a radio node or a base station are sometimes referred to generically as a ‘computer network device.’

As described further below with reference to FIG. 52, source 110, measurement device(s) 112, computer system 116, and/or computer system 136 may include subsystems, such as a networking subsystem, a memory subsystem and a processor subsystem. In some embodiments, source 110, measurement device(s) 112 and/or computer system 116 may include radios (not shown) in the networking subsystems. More generally, source 110, measurement device(s) 112 and/or computer system 116 can include (or can be included within) any electronic devices with the networking subsystems that enable source 110, measurement device(s) 112 and/or computer system 116 to wirelessly communicate with one or more other electronic devices. This wireless communication can comprise transmitting access on wireless channels to enable electronic devices to make initial contact with or detect each other, followed by exchanging subsequent data/management frames (such as connection requests and responses) to establish a connection, configure security options, transmit and receive frames or packets via the connection, etc.

During the communication in FIG. 1, source 110, measurement device(s) 112 and/or computer system 116 may wired or wirelessly communicate while: transmitting access requests and receiving access responses on wireless channels, detecting one another by scanning wireless channels, establishing connections (for example, by transmitting connection requests and receiving connection responses), and/or transmitting and receiving frames or packets (which may include information as payloads).

For example, wireless signals (not shown) may be transmitted by a radio in computer system 116. Notably, the radio computer system 116 may transmit information (such as one or more packets or frames) using wireless signals. These wireless signals are received by a radio (not shown) in, e.g., source 110 or measurement device(s) 112. This may allow computer system 116 to communicate information to source 110 and/or measurement device(s) 112. Note that the wireless signals may convey one or more packets or frames.

In the described embodiments, processing a packet or a frame in source 110, measurement device(s) 112 and/or computer system 116 may include: receiving the wireless signals with the packet or the frame; decoding/extracting the packet or the frame from the received wireless signals to acquire the packet or the frame; and processing the packet or the frame to determine information contained in the payload of the packet or the frame.

Note that the wired and/or the wireless communication in FIG. 1 may be characterized by a variety of performance metrics, such as: a data rate for successful communication (which is sometimes referred to as ‘throughput’), an error rate (such as a retry or resend rate), a mean-squared error of equalized signals relative to an equalization target, intersymbol interference, multipath interference, a signal-to-noise ratio, a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’).

In some embodiments, wireless communication between components in FIG. 1 uses one or more bands of frequencies, such as, but not limited to: 900 MHZ, 2.4 GHz, 5 GHZ, 6 GHZ, 7 GHz, 60 GHz, the Citizens Broadband Radio Spectrum or CBRS (e.g., a frequency band near 3.5 GHZ), and/or a band of frequencies used by LTE or another cellular-telephone communication protocol or a data communication protocol. Note that the communication between electronic devices may use multi-user transmission (such as orthogonal frequency division multiple access or OFDMA) r multiple input, multiple output (MIMO) communication.

Although we describe the network environment shown in FIG. 1 as an example, in alternative embodiments, different numbers or types of electronic devices may be present. For example, some embodiments comprise more or fewer electronic devices. As another example, in another embodiment, different electronic devices are transmitting and/or receiving packets or frames.

As discussed previously, it can be difficult to present and/or large amounts of data, such as data acquired using system 100. In order to address this problem, computer system 136 may performing the computing techniques.

Notably, computer system 136 may archive or store measured data acquired using system 100. In some embodiments, computer system 136 may store additional information, such as medical records (such as a medical history of one or more individuals), other measurement or test results (such as blood test results, genetics test results, vital signs, and/or results of other non-invasive characterization techniques for the one or more individuals), etc. Moreover, computer system 136 may provide analytical services. While or after data is acquired using system 100, a user (such as a physician or a healthcare provider) of computer 138 may access the measured data and/or additional data stored by computer system 136.

For example, the user may interact with a user interface displayed on computer 138 using a user-interface device, such as: a keyboard, a mouse, a touchscreen, a trackpad, a stylus, a voice-recognition engine, etc. This user-interface activity information may specify content selected by the user.

Then, computer 138 may provide information specifying the selected content (such as the user-interface activity information) to computer 136. In response to receiving the user-interface activity information or the information specifying the selected content, computer system 136 may dynamically determine related content based at least in part on one or more prior expert selections that are associated with the selected content and/or one or more predefined or predetermined relationships with the selected content.

Note that the selected content may include an image associated with a non-invasive characterization technique. Notably, the non-invasive characterization technique may include: MRI, computed tomography, x-ray imaging and/or ultrasound. In some embodiments, the image may correspond to a portion of a human body. For example, the selected content may include images associated with a scan of an individual. Moreover, the related content may include a measurement associated with a different measurement technique than the non-invasive characterization technique. However, in other embodiments, the related content may include one or more additional measurements associated with the non-invasive characterization technique, such as one or more additional measurements of the portion or another portion of the human body.

Furthermore, the selected content may include or may be associated with a biomarker, and the one or more predefined or predetermined relationships may be between the biomarker and one or more additional biomarkers. Additionally, the one or more predefined or predetermined relationships may be based at least in part on: genetics information, chemistry, a behavior, an environmental factor, a disease (such as cholesterol and heart disease), a comorbidity and/or a biological pathway (e.g., the selected content and the related content may share the same or a similar biological pathway). In some embodiments, the one or more predefined or predetermined relationships may be based at least in part on medical literature.

Moreover, the content may be associated with a patient, and the one or more prior expert selections may be by a physician and may be associated with one or more different patients from the patient. Note that the patient and the one or more different patients may share one or more characteristics or attributes. For example, the patient and the one or more different patients may have a disease (such as the patient and the one or more different patients may have been diagnosed as having the disease), a risk of having the disease (which may be based at least in part on a medical history of an individual or a potential diagnosis by a physician or healthcare provider), and/or may be members of a demographic group or a common population.

Next, computer system 136 may provide the selected content and the determined related content. For example, computer system 136 may provide the selected content and the determined related content to computer 138, which displays the selected content and the determined related content on a display. Moreover, providing the determined related content may include providing a user interface with user-interface features (such as one or more virtual icons) corresponding to the determined related content. Note that the user may interact with the user interface using the user-interface device to select or modify the presentation of the content and/or the related content (such as to change a presented image, a magnification, a viewing angle, a slice in a sequence of slices, etc.).

In some embodiments, the dynamic determining may be performed using a pretrained predictive model. For example, the selected content and one or more of the aforementioned features (such as the image, information specifying the portion of the human body, the genetics information, the chemistry, the behavior, the environmental factor, the disease, the risk of having the disease, the comorbidity, the biological pathway, the medical literature, information specifying the demographic group, information specifying the population, the one or more characteristics or attributes, etc.) may be input to the pretrained predictive model, and information specifying the related content may be output by the pretrained predictive model.

Moreover, the pretrained predictive model may include a machine-learning model, such as a machine-learning model trained using a supervised-learning technique or an unsupervised learning technique (such as clustering). In some embodiments, a machine-learning model may include: a support vector machine (SVM), a classification and regression tree, logistic regression, LASSO, LASSO logistic regression, linear regression, nonlinear regression, pattern recognition, a Bayesian technique, and/or another (linear or nonlinear) supervised-learning technique. Alternatively or additionally, the pretrained predictive model may include a neural network, such as a convolutional neural network, a long short term memory (LTSM) neural network, an autoencoder, etc. Note that the neural network may include multiple layers with nodes or neurons. A given node may have an associated weight and one or more activation functions (such as a rectified linear activation function or ReLU, a leaky ReLU, an exponential linear unit or ELU activation function, a parametric ReLU, a tanh activation function, or a sigmoid activation function) for each parameter input to the given node.

The pretrained predictive model may have been trained using a training dataset, such as a training dataset of sequences of content and related content viewed or used by experts (such as physicians or healthcare providers) when reviewing measured data (such as results of one or more non-invasive characterization techniques) for a group of individuals or patients (which may be different from a current individual or patient associated with the content, or which may include the current individual or patient). For example, the training dataset may include at least 10-1,000 ‘encounters’ during which one or more experts select content and related content. Consequently, in some embodiments, the pretrained predictive model may implement collaborative filtering.

In some embodiments, the pretrained predictive model may be dynamically retrained over time using user selections (or the lack of user selections) in the user interface that presents the related content as feedback about the accuracy of the dynamically determined related content. This dynamic retraining may be performed periodically (such as daily, weekly, etc.), on-demand (such as when requested by a user) or as-needed (such as based at least in part on the accuracy or a confidence interval of the dynamically determined related content). For example, when the accuracy is less than a predefined threshold (such as 1, 5, 10 or 25%) or the confidence interval is greater than a second predefined threshold (such as 1, 5, 10 or 25%), the pretrained predictive model may be retrained.

While the preceding discussion illustrated the computing techniques using a centralized approach, in other embodiments the computing techniques may be, at least in part, implemented in a decentralized manner. For example, at least some of the operations in the computing techniques may be performed by computer system 116, computer system 136 and/or computer 138. Notably, the computing techniques may be performed by a stand-alone program or a Web-browser plugin on computer 138, instead of using a client-server architecture or a cloud-based architecture. Thus, the computing techniques may, at least in part, be implemented in a centralized manner and/or distributed manner.

In these ways, the computing techniques may allow the user to quickly, efficiently and accurately access, view and/or use relevant content that is related to selected content in large datasets. This capability may provide useful information to the user that improves monitoring of an individual's health, diagnosing of disorders or diseases, selecting treatment(s) and/or assessing outcomes of the treatment(s). Consequently, the computing techniques may improve the user experience when using computer system 136 and may improve patient health.

Moreover, while system 100 is illustrated as having particular components, in other embodiments system 100 may have fewer or more components, two or more components may be combined into a single component, and/or positions of one or more components may be changed.

We now describe embodiments of a method. FIG. 2 presents a flow diagram illustrating an example of a method 200 for determining model parameters associated with a sample. This method may be performed by a system (such as system 100 in FIG. 1), or one or more components in a system (such as source 110, measurement device 114 and/or computer system 116).

During operation, a source in the system may apply, to the sample, an excitation (operation 210), where the excitation has at least a wavelength and an intensity or a flux. For example, the excitation may include one of: electromagnetic radiation, a radio-frequency wave, a particle beam, a sound wave, a magnetic field, and/or an electric field. Therefore, the excitation may include at least one of: an electromagnetic beam in an x-ray band of wavelengths, a neutron beam, an electron beam, an electromagnetic beam in an optical band of wavelengths, an electromagnetic beam in an infrared band of wavelengths, a sound wave in an ultrasound band of wavelengths, a proton beam, an electric field associated with an impedance measurement device, a radio-frequency wave associated with a magnetic-resonance apparatus, and/or a magnetic field associated with a susceptibility measurement device.

Then, a measurement device in the system may measure a response (operation 212) associated with the sample to the excitation. For example, the measurement device may include at least one of: an x-ray detector, a neutron detector, an electron detector, an optical detector, an infrared detector, an ultrasound detector, a proton detector, the magnetic-resonance apparatus, the impedance measurement device and/or the susceptibility measurement device. Note that the measured response may include a time-domain response of the sample and may be other than or different from an image.

Moreover, the system may compute, using the measured response and information specifying the excitation as inputs to a predetermined predictive model, model parameters (operation 214) on a voxel-by-voxel basis in a forward model with multiple voxels that represent the sample. The forward model may simulate response physics occurring within the sample to a given excitation with a given wavelength and a given intensity or a given flux, that are selected from a range of measurement conditions that includes the excitation, the wavelength and the intensity or the flux, and at least a different wavelength and a at least a different intensity or a different flux. Furthermore, the forward model may be a function of the excitation, the model parameters of the multiple voxels, and differential or phenomenological equations that approximates the response physics.

Note that the predetermined predictive model may include a machine-learning model and/or a neural network. In some embodiments, the predetermined predictive model includes a personalized predictive model that corresponds to an individual.

Next, the system may determine an accuracy of the model parameters (operation 216) by comparing at least the measured response and a calculated predicted value of the response using the forward model, the model parameters and the excitation.

Additionally, when the accuracy exceeds a predefined value (operation 218), the system may provide the model parameters (operation 220) as, e.g., an output to a user, to another electronic device, to a display and/or to the memory.

Thus, when the accuracy exceeds the predefined value (operation 218), the model parameters may be computed in a single pass without further iteration. Consequently, the model parameters having an accuracy exceeding the predefined value may be computed using fewer iterations with the predetermined predictive model than in the iterative approach without the predetermined predictive model.

Alternatively, when the accuracy is less than the predefined value (operation 218), the system may: calculate, using information specifying the excitation and the accuracy as inputs to a second predetermined predictive model, a revised excitation (operation 222) that has at least a revised wavelength, a revised intensity or a revised flux; and repeat (operation 224) the applying, measuring, computing and determining operations with the revised excitation instead of the excitation. Note that the second predetermined predictive model may include a machine-learning model and/or a neural network.

In some embodiments, the system optionally performs one or more optional additional or alternative operations. For example, when the accuracy is less than the predefined value (operation 218), the system may: add the excitation and the measured response to a training dataset; and determine, using the training dataset, a revised instance of the predictive model.

Additionally, the system may classify or segment one or more anatomical structures in the sample using the model parameters and a third predictive model. For example, the third predetermined predictive model may include a machine-learning model and/or a neural network.

Moreover, the system may train the predictive model using a simulated dataset computed using the forward model, a range of model parameters and a range of excitations.

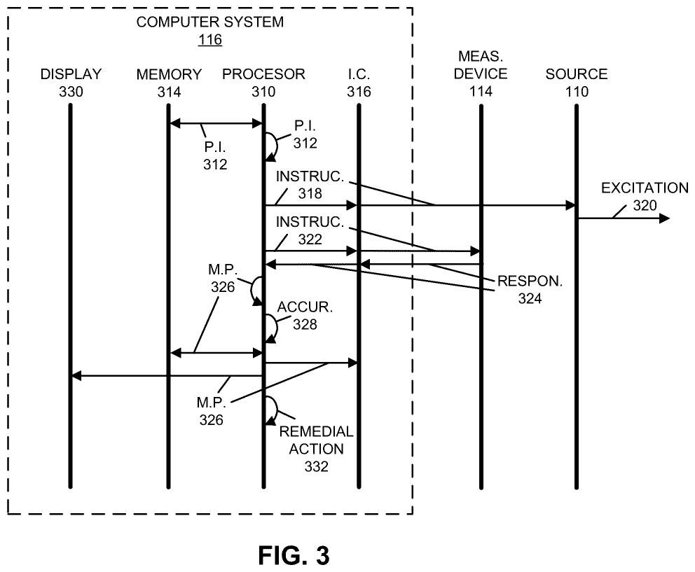

FIG. 3 presents a drawing illustrating an example of communication among components in system 100 (FIG. 1). Notably, processor 310 in computer system 116 may execute program instructions (P.I.) 312 stored in memory 314. When processor 310 executes program instructions 312, processor 310 may perform at least some of the operations in the computation techniques.

During the computation techniques, processor 310 may provide instruction 318 to interface circuit (I.C.) 316. In response, interface circuit 316 may provide instruction 318 to source 110, e.g., in one or more packets or frames. Moreover, after receiving instructions 318, source 110 may apply, to the sample, an excitation 320.

Then, processor 310 may provide instruction 322 to interface circuit 316. In response, interface circuit 316 may provide instruction 322 to measurement device 114, e.g., in one or more packets or frames. Furthermore, after receiving instructions 322, measurement device 114 may measure a response 324 associated with the sample to excitation 320. Next, measurement device 114 may provide measured response 324 to computer system 116, e.g., in one or more packets or frames.

After receiving measured response 324, interface circuit 316 may provide measured response 324 to processor 310. Then, using measured response 324 and information specifying excitation 320 as inputs to a predetermined predictive model, processor 310 may compute model parameters (M.P.) 326 on a voxel-by-voxel basis in a forward model with multiple voxels that represent the sample.

Additionally, processor 310 may determine an accuracy 328 of the model parameters by comparing at least measured response 324 and a calculated predicted value of the response using the forward model, model parameters 326 and excitation 320. When accuracy 328 exceeds a predefined value, processor 310 may provide the model parameters 326 as, e.g., an output to a user, to another electronic device (via interface circuit 316), to a display 330 and/or memory 314.

Otherwise, when the accuracy is less than the predefined value, processor 310 may perform a remedial action 332. For example, processor 310 may: calculate, using information specifying excitation 320 and accuracy 328 as inputs to a second predetermined predictive model, a revised excitation; and repeat the applying, measuring, computing and determining operations with the revised excitation instead of excitation 320. Alternatively or additionally, processor 310 may: add excitation 320 and measured response 324 to a training dataset; and determine, using the training dataset, a revised instance of the predictive model.

We now describe embodiments of predictive models. For example, a predictive model may include a machine-learning model, such as a supervised-learning model or an unsupervised learning technique (such as clustering). In some embodiments, a machine-learning model may include: a support vector machine (SVM), a classification and regression tree, logistic regression, LASSO, LASSO logistic regression, linear regression, nonlinear regression, pattern recognition, a Bayesian technique, and/or another (linear or nonlinear) supervised-learning technique.

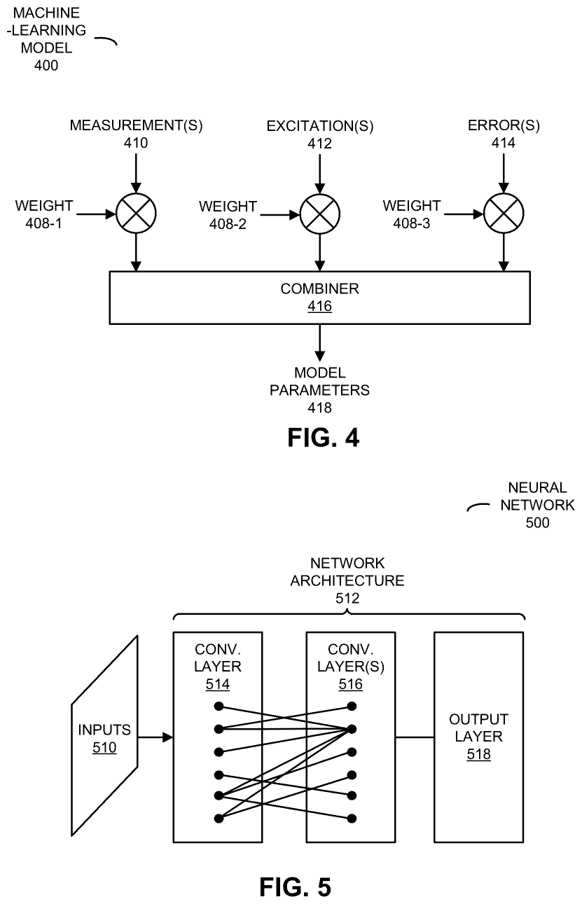

FIG. 4 presents a drawing illustrating an example of a machine-learning model 400. In this machine-learning model, a weighted (using weights 408) linear or nonlinear combination 416 of measurements 410, one or more corresponding excitations 412 and one or more errors 414 between the one or more measurements 410 and one or more predicted responses determined using a forward model, a current instance of the model parameters of voxels in the forward model, and the one or more excitations 412 is used to compute a revised instance of model parameters 418. Thus, in some embodiments, predictive model 400 is used in conjunction with forward model to iteratively modify instances of the model parameters until an accuracy of the predicted response is less than a predefined value (i.e., a convergence criterion is achieved). However, in some embodiments, a machine-learning model may be used to determine the model parameters in one pass, i.e., in an open-loop manner.

Alternatively or additionally, a predictive model may include a neural network. Neural networks are generalized function approximators. For example, techniques such as deep learning typically use previous examples as inputs. In general, it is not possible for these machine-learning models to determine the actual function they are trying to approximate because there is no reference point for them to use to estimate the error in their predictions. In particular, it can be difficult for a neural network to make predictions based on an input that is very different from the examples it was trained on. In this regard, a neural network can be thought of as a lossy compute compression engine.

However, by training a neural network using a wide variety of excitations, measured responses and corresponding model parameters, the neural network can provide the model parameters (or initial estimates of the model parameters) for a forward model that simulates the physics of a response of a sample to an excitation. Because neural networks are effective approximations/compressions, they may execute faster on the same inputs with less computational power required. Moreover, because the functions are known in the forward model, the responses can be computed and the accuracy of the predictions can be assessed (as opposed to using an approximation). Therefore, the computation techniques can be used to determine when its predictions are unreliable. In particular, as discussed previously for FIG. 4, a neural network may be used in conjunction with forward model to iteratively modify instances of the model parameters until an accuracy of the predicted response is less than a predefined value (i.e., a convergence criterion is achieved). In some embodiments, however, a neural network may be used to determine the model parameters in one pass, i.e., in an open-loop manner.

FIG. 5 presents a drawing illustrating an example of a neural network 500. This neural network may be implemented using a convolutional neural network or a recurrent neural network. For example, neural network 500 may include a network architecture 512 that includes: an initial convolutional layer 514 that provides filtering of inputs 510 (such as one or more measurements and a difference or an error between the one or more measurements and one or more predicted responses determined using a forward model, a current instance of model parameters and an excitation); an additional convolutional layer(s) 516 that apply weights; and an output layer 518 (such as a rectified linear layer) that performs selection (e.g., selecting a revised instance of the model parameters). Note that the details with the different layers in neural network 500, as well as their interconnections, may define network architecture 512 (such as a directed acyclic graph). These details may be specified by the instructions for neural network 500. In some embodiments, neural network 500 is reformulated as a series of matrix multiplication operations. Neural network 500 may be able to handle the real-world variance in 1 million inputs or more. Note that neural network 500 may be trained using a deep-learning technique or a GAN. In some embodiments of machine-learning model 400 (FIG. 4) and/or neural network 500, a current instance of the model parameters is used as an input.

In some embodiments, a large convolutional neural network may include 60 M parameters and 650,000 neurons. The convolutional neural network may include eight learned layers with weights, including five convolutional layers and three fully connected layers with a final 1000-way softmax or normalized exponential function that produces a distribution over the 1000 class labels for different possible model parameters. Some of the convolution layers may be followed by max-pooling layers. In order to make training faster, the convolutional neural network may use non-saturating neurons (such as a local response normalization) and an efficient dual parallelized GPU implementation of the convolution operation. In addition, in order to reduce overfitting in the fully-connected layers, a regularization technique (which is sometimes referred to as ‘dropout’) may be used. In dropout, the predictions of different models are efficiently combined to reduce test errors. In particular, the output of each hidden neuron is set to zero with a probability of 0.5. The neurons that are ‘dropped out’ in this way do not contribute to the forward pass and do not participate in backpropagation. Note that the convolutional neural network may maximize the multinomial logistic regression objective, which may be equivalent to maximizing the average across training cases of the log-probability of the correct label under the prediction distribution.

In some embodiments, the kernels of the second, fourth, and fifth convolutional layers are coupled to those kernel maps in the previous layer that reside on the same GPU. The kernels of the third convolutional layer may be coupled to all kernel maps in the second layer. Moreover, the neurons in the fully connected layers may be coupled to all neurons in the previous layer. Furthermore, response-normalization layers may follow the first and second convolutional layers, and max-pooling layers may follow both response-normalization layers as well as the fifth convolutional layer. A nonlinear model of neurons, such as Rectified Linear Units, may be applied to the output of every convolutional and fully-connected layer.

In some embodiments, the first convolutional layer filters a 224×224×3 input image with 96 kernels of size 11×11×3 with a stride of four pixels (this is the distance between the receptive field centers of neighboring neurons in a kernel map). Note that the second convolutional layer may take as input the (response-normalized and pooled) output of the first convolutional layer and may filter it with 256 kernels of size 5×5×48. Furthermore, the third, fourth, and fifth convolutional layers may be coupled to one another without any intervening pooling or normalization layers. The third convolutional layer may have 384 kernels of size 3×3×256 coupled to the (normalized, pooled) outputs of the second convolutional layer. Additionally, the fourth convolutional layer may have 384 kernels of size 3×3×192, and the fifth convolutional layer may have 256 kernels of size 3×3×192. The fully-connected layers may have 4096 neurons each. Note that the numerical values in the preceding and the remaining discussion below are for purposes of illustration only, and different values may be used in other embodiments.

In some embodiments, the convolutional neural network is implemented using at least two GPUs. One GPU may run some of the layer parts while the other runs the remaining layer parts, and the GPUs may communicate at certain layers. The input of the convolutional neural network may be 150,528-dimensional, and the number of neurons in the remaining layers in the convolutional neural network may be given by 253, 440-186, 624-64, 896-64, 896-43, and 264-4096-4096-1000.

We now describe embodiments of a forward model. This forward model may be a 3D model of voxels in a portion of a sample (such as an individual), and may include model parameters in the Bloch equations for each of the voxels. In particular, with a quasi-static magnetic field B₀ along the z axis, the Bloch equations are

$$\\ \frac{{dM}_{x} ( t )}{dt} = γ \cdot {( \vec{M} ( t ) ⊗ \vec{B} ( t ) )}_{x} - \frac{{M}_{x} ( t )}{{T}_{2}} ,$$

$$\\ \frac{{dM}_{y} ( t )}{dt} = γ \cdot {( \vec{M} ( t ) ⊗ \vec{B} ( t ) )}_{y} - \frac{{M}_{y} ( t )}{{T}_{2}} , and$$

$$\\ \frac{{dM}_{𝓏} ( t )}{dt} = γ \cdot {( \vec{M} ( t ) ⊗ \vec{B} ( t ) )}_{𝓏} - \frac{{M}_{𝓏} ( t ) - {M}_{0}}{{T}_{1}} ,$$

where γ is the gyromagnetic ratio, ⊗ denotes a vector cross product and \vec{B}(t)=(Bₓ(t), B_{y}(t), B₀+ΔB_{z}(t)) is the magnetic field experienced by a type of nuclei in the sample. The model parameters in the Bloch equations may include T₁, T₂, a density of a type of nuclei, diffusion, velocity/flow, temperature, magnetic susceptibility, etc. Note that there may be different model parameters for different types of nuclei for each of the voxels. Moreover, note that the Bloch equations are a semi-classical, macroscopic approximation to the dynamic response of the magnetic moments of the type of nuclei in the sample to a time-varying magnetic field. For example, there may be 67 M cells in a 1 mm³ voxel.

In principle, the solution space for the model parameters in the Bloch equations for the sample may be underdetermined, i.e., there may be significantly more model parameters to be determined than there are observations with which to specify or constrain the model parameters. Therefore, when training a predictive model or determining the model parameters using the predictive model (such as using computations in a machine-learning model or in a layer in a neural network), the computation techniques may leverage additional information to constrain or reduce the dimensionality of the problem. For example, an aspect of the anatomy of the sample may be determined using other imaging techniques, such as computed tomography, x-ray, ultrasound, etc. Moreover, regions that do not look like (i.e., that has very different measurements, e.g., different measured MR signals) than a targeted type of tissue (such as heart tissue) may be excluded from the forward model (such as by setting the model parameters to zero in these regions). In this way, e.g., regions that include air may be excluded. Other constraints in the forward model may include: thermodynamic constraints on heat flow (from hot to cold) for perfusion or MRT to quantify metabolism. In addition, the predictive model may be trained using measurements at different magnetic-field strengths B₀ (which may provide similar information to pseudorandom RF pulse sequences) using different RF pulse sequences and/or different MR techniques, which may reduce the ratio of model parameters to observations, thereby simplifying the training of the predictive model.

Alternatively or additionally, tissue that deviates significantly from a predicted or simulated response (such as predicted MR signals) based on previous MR measurements or scans (e.g., anomalies or changes) may become the focus of the forward model, such as by using a contour map (e.g., a cubic spline) to bound the regions (or specify a boundary of the regions) where there are significant differences. In some embodiments, when training the predictive model or determining the model parameters using the predictive model (such as using computations in a machine-learning model or in a layer in a neural network), the difference or error between measurements and simulated or predicted responses may be represented using one or more level-set functions, and the boundaries of regions with errors exceeding a threshold value may be determined based on the intersection of a plane corresponding to the threshold value and the one or more level-set functions.

In some embodiments, a layer in a neural network may compute first and second derivatives along a surface(s) of model-parameter solutions in the sample. (In order to facilitate calculation of a derivative, the model parameters may be represented using one or more level-set functions.) A set of voxels along the line where the first derivative is zero may be identified. This set of voxels may be fit using a cubic spline with a minimum error between the voxel positions and the cubic spline. This fitting operation may be repeated at all the boundaries in the model-parameter-solution space. Moreover, the largest continuous surface within the boundary defined by the cubic splines may be determined and the model-parameter-solution calculation may be repeated to determine a new continuous surface that is within the previous continuous surface. This generalized framework may minimize the error across intra-voxel volumes, thereby improving the agreement between the measurements and the simulated or predicted responses based on the forward model.

For example, a neural network may solve the inverse problem using a Jacobian matrix of the model parameters for the voxels in the forward model and Newton's method to modify the model parameters for the voxels in successive layers based on how perturbations in the model parameters affect the difference or error between the measurements and the predicted responses.

In some embodiments, if a portion of the sample included one voxel, there may be 4-10 model parameters (which specify a forward model) that need to be determined for a particular type of tissue. If the voxel includes M types of tissue, there may be 4M-10M the model parameters that need to be determined for the particular type of tissue. As the number of voxels increases, this can appear to be a daunting problem.

However, because different types of nuclei have different Larmor frequencies, the spatial distribution of the types of nuclei and their local concentrations may be determined from the measurements. Then, a predefined anatomical template for the human body (or a portion of the human body), with associated initial model parameters for the forward model, may be scaled to match the spatial distribution of the types of nuclei and their local concentrations. For example, predetermined or predefined ranges for the model parameters in different types of tissue may be used to determine for the initial model parameters. In some embodiments, the initial model parameters are based on model parameters associated with previous measurements or MR scans.

Next, a look-up table with simulated or predicted responses (generated using one or more forward models) as a function of associated model parameters and excitations may be used modify the initial model parameters or to compute model parameters for voxels in the sample. For example, simulated or predicted responses that are similar to measurements may be identified, and the differences or errors between these simulated or predicted responses and the measurements may be used to guide interpolation between the model parameters in the look-up table.

In some embodiments, for a type of tissue (such as a particular organ), the model parameters determined using different layers in a neural network may be iteratively refined as the size of the voxels is progressively decreased (and, thus, the number of voxels is increased) in the different layers. This analysis may be driven by the error between the measurements and simulated or predicted responses using the forward model. Progressing through successive layers in a neural network, the focus may be on the residual regions with errors that are larger than a convergence or an accuracy criterion. For example, the model parameters for the forward model in a layer in a neural network may be based on measurements at one magnetic-field strength and then the error may be determined based on the predicted response of the forward model at another magnetic-field strength. Furthermore, note that initially the predictive model or the forward model may assume that there is no contribution or interaction between different voxels. However, as the error and the voxel size are reduced, such contributions and/or interactions may be included in subsequent layers in a neural network. In some embodiments, when there are multiple candidate model-parameter solutions (having similar errors) to the inverse problem for a layer in a neural network, at least some of these candidates may be kept for use in a subsequent layer (i.e., a unique model-parameter solution may not be identified at this point). Alternatively, if there is no unique parameter solution within a desired error range (such as less than 50, 25, 10, 5 or 1%), the best (least-error) model-parameter solution may be kept. In addition, when there is no model-parameter solution within the desired error range, the second predictive model may be used to modify the excitation and additional measurement(s) may be performed.

Thus, the inverse problem of determining the model parameters based on measurements may be ‘solved’ using a predictive model that provides model parameters that minimize the error or difference between the measurements and simulated or predicted responses that are generated based on the forward model, the model parameters and an excitation. In some embodiments, the inverse problem is solved using one or more analysis techniques, including: a least-squares technique, a convex quadratic minimization technique, a steepest descents technique, a quasi-Newton technique, a simplex technique, a Levenberg-Marquardt technique, simulated annealing, a genetic technique, a graph-based technique, another optimization technique and/or Kalman filtering (or linear quadratic estimation).

Note that the training of a predictive model may use dynamic programming. In particular, the training problem may be divided up and performed by multiple computers in parallel, e.g., in a cloud-based computing system. For example, a particular thread may attempt to solve the inverse problem for particular measurement conditions. Multiple potential model-parameter solutions generated by the computers (or processors) may be combined (e.g., using linear superposition) to determine an error metric that is minimized using the one or more analysis techniques.

Moreover, as described previously, the inverse problem may be solved iteratively by a predictive model (such as machine-learning model or a neural network) by first attempting to find suitable model parameters (e.g., model parameters that minimize the error between measurements and simulated or predicted responses) for the forward model using a coarse voxel size and then progressively finding suitable parameters with smaller voxel sizes in subsequent layers or stages of the calculation. Note that the final voxel size used in this iterative procedure (or a suitable range of voxel sizes, because the voxel size may not be fixed in some embodiments) may be determined based on the gyromagnetic ratio of a type of nuclei being scanned. Furthermore, the voxel size or locations may also be chosen so that a voxel is evenly portioned into a set of subvoxels, or so that there is certain amount of overlap with preview voxel sizes to effectively ‘oversample’ the overlapping region and potentially further localize where an MR signal originates. This last technique may be akin to shifting the entire gradient system in one or more dimensions by a distance dx that is less than a characteristic length of the voxels (such as a length, a width or a height of the voxels). In some embodiments, the voxel size in the predictive model or the forward model is smaller than that used in the measurements (i.e., the predictive model or the forward model may use a super-resolution technique). For example, there may be 512×512 voxels or 1024×1024 voxels at a magnetic-field strength of 3T. Note that the voxel size may be less than 0.253 mm³.

We now describe embodiments of a technique for segmenting different types of tissue, which may be used by the third predictive model (such as a neural network). Define a dictionary Dmr of measured time-sampled MR trajectories (or vectors) in a multi-dimensional parameter space for different types of tissue dj (for j=1 to n) such that a measured MR signal y_{obv} for a voxel can be expressed as

$$\\ {y}_{obv} = {\sum }_{j = 1}^{n} {α}_{j} \cdot {d}_{j} + ε ,$$

where αⱼ are normalized weights (i.e.,

$$\\ {\sum }_{j = 1}^{n} {α}_{j} = 1 )$$

and ε is an error (i.e., ε=(yⱼ, αⱼ), for j=1 to n. This may define an intra-voxel linear equation problem. A generalized inter-voxel problem may model a set of voxels (such as a cube with 27 voxels) as a graph G. Note that each voxel in the set may have 26 edges to eight adjacent voxels. A parameter solution to the inverse problem may be defined as one that minimizes the error.

Consider the case of two adjacent voxels u and v. The intra-voxel linear equations U_{y} and V_{y} need to be solved at both u and v. There are several possible outcomes. First, U_{y} and V_{y} may have unique model-parameter solutions (where a ‘unique model-parameter solution’ may be a best fit to an existing forward model, i.e., with an error or difference vector that is less than a convergence or an accuracy criterion) and the analysis may be finished. Alternatively, U_{y} may have a unique model-parameter solution but not V_{y}. It may be possible that the model-parameter solution for U_{y} imposes a constraint on V_{y} such that V_{y} has a single model-parameter solution, in which case the analysis may be finished. However, neither U_{y} and V_{y} may have unique model-parameter solutions, in which case combining the systems of equations (i.e., effectively increasing the voxel size) may yield a unique model-parameter solution. Moreover, neither U_{y} and V_{y} may have any model-parameter solutions, in which case the intra-voxel problem cannot be solved without further constraints.

In the last case, it may be possible to look at an adjacent voxel w, i.e., series voxels u, v and w, with the corresponding intra-voxel linear equations U_{y}, V_{y} and W_{y} need to be solved at u, v and w. Note that the intra-voxel linear equations V_{y} and W_{y} reduce to the previous case. When the intra-voxel linear equations do not reduce to the previous case, this paring operation can be applied recursively until it does and then the intra-voxel linear equations can be solved as described previously.

In general, this analysis technique may be isomorphic to the problem of fitting a 3D surface (or volume) to minimize the error. One challenge in this regard is that it assumes that all adjacent volumes have an equal effect on the model-parameter solution αⱼ that minimizes the error.

The minimization of the error may initially assume that there is no inter-voxel contribution (i.e., that the voxels are independent). Subsequently, inter-voxel contributions may be included. In particular, considering adjacent voxel volumes, there are two distinct classes. Volumes that share a surface and volumes that only share a 1D edge. The minimization function can be improved by weighting the error contribution at voxel u at the center of the relative co-ordinate system. If the effect on the error is proportional to r⁻² (where r is the distance between center points of voxels) and assuming 1 mm isotropic voxels in the weightings, the minimization or fitting problem with inter-voxel contributions can be expressed as

$$\\ min ( error ( y ( 0 , 0 , 0 ) , α ( 0 , 0 , 0 ) + \frac{1}{{( 1 )}^{2}} {\sum }_{k = 1}^{m} error ( {y}_{k} , {α}_{k} ) + \frac{1}{{( \sqrt{2} )}^{2}} {\sum }_{l = 1}^{p} error ( {y}_{l} , {α}_{l} ) ,$$

where the summation over k is for adjacent voxels sharing a common surface (i.e., (−1, 0, 0), (1, 0, 0), (0, −1, 0), (0, 1, 0), (0, 0, −1) and (0, 0, 1)) and the summation over 1 is for a remainder of adjacent voxels sharing a common edge. The assumption in the analysis is that the most difficult place to fit or determine model-parameter solutions is at discontinuities or interfaces between different tissues. Consequently, during the computation techniques, analysis engine 122 (FIG. 1) may solve these locations first and then may solve the remaining locations.

Alternatively, because the magnetic contribution from neighboring voxels is proportional to r², given a sphere of radius R from the center of a primary or central voxel in the minimization problem, surrounding voxels may be weighted based on the how much the sphere expands into the volume of the adjacent voxels (and, thus, based on how strong their inter-voxel contribution is estimated to be). For example, there may be three different weights that need to be assigned, including: a weight for voxels that share a 2D surface, a weight for voxels that share a 1D line, and a weight for voxels that share a 0D point. Because there may not be a uniform tissue distribution within each voxel, the weights may be dynamically adjusted to model different kinds of distributions inside each voxel in order find the distributions that minimize the error. This may provide the ability to identify multiple MR signatures within a single voxel for different types of tissue. Note that, as computational power increases, the accuracy of the third predictive model may increase and the analysis technique used to solve the minimization problem (and, thus, the inverse problem) may be modified.

Thus, in embodiments where the forward model of a voxel depends on the forward models of surrounding or neighboring voxels, the forward model of a voxel may be computed using 2^{nd} or N^{th}-order effects. For example, if there are N 1^{st}-order forward models (where N is an integer), there may be as many as N!/(N−27)! 2^{nd}-order forward models (if all the voxels interact with each other). In some embodiments, locality is used to simplify the inverse problem. In this way, a forward model may be generated by incorporating how the forward models in adjacent voxels effect the forward model in a primary (central) or 1^{st}-order voxel.

In some embodiments, a dithering technique is used to overcome the arbitrary locations of the voxels relative to the distribution of types of tissue in the body. In particular, there may be two or more types of tissue in a voxel because of the arbitrary voxel placement or the current voxel size. This may significantly change the forward model parameters for this voxel. This may suggest that there is more than one forward model needed for the voxel. In order to confirm this, the voxels may be displaced by a distance dx (which is a fraction of the voxel length, width or height) and the forward model parameters may be determined again (e.g., using the predictive model). In the processes, the tissue distribution may be determined. Consequently, this approach may effectively increase the spatial resolution in the analysis without changing the voxel size.

FIG. 6 presents a drawing illustrating an example of classification or segmentation of one or more anatomical structures 600. Notably, FIG. 6 illustrates identifying or segmenting an organ 610 based at least in part on discontinuous changes in T₁ and T₂ at voxel boundaries.

While the preceding discussion illustrated the computation techniques using MR techniques, these approaches may be generalized to a measurement system that is able to physically model and measure a sample in real-time using a wide variety of characterization techniques. In general, the computation techniques can use a combination of mechanical and/or electromagnetic waves to ‘perturb’ or ‘excite’ the volume being scanned in order to evaluate the correctness of a prediction in terms of how the volume responds to the perturbation. This also includes the ability for the system to simulate itself and any part of the environment in which the system is located that could affect the correctness or accuracy of the forward model the system is trying to generate to describe the volume being scanned or measured.

Note that the different characterization techniques may provide tensor-field mapping and the ability to detect anomalies in tensor fields. These maps can be images or quantitative tensor field maps, and each of the characterization techniques may provide a visualization of a different type of tensor field map captured with different type of measurements. By looking at or considering two or more of these maps, of the system may have access to orthogonal information.

Thus, the system may provide a way to capture, in real-time or near real-time, higher-order or hyper-dimensional pseudo or hybrid tensors or matrices at each voxel in 3D space. Using electromagnetic and/or mechanical perturbations or excitations, the system may use different characterization techniques to measure disturbances and responses, and then to simulate the responses.

The result of this characterization may be a (4+N)D (three spatial dimensions, one time dimension, and up to N measurement dimensions at each point in space) quantitative model of the volume being scanned. Note that the (4+N)D quantitative model may be projected onto an arbitrary subset of the full (4+N)D space, including 2D or 3D images.

In some embodiments, the use of multidimensional data and models provides enhanced diagnostic accuracy (i.e., a lower false-positive rate) relative to conventional MRI approaches, even if a larger voxel size is used. Thus, the computation techniques may allow improved diagnostic accuracy with a larger voxel size (or a weaker external magnetic field) than would be needed in conventional MRI. However, as noted previously, the computation techniques may be used with a wide variety of measurement techniques separately from or in addition to MRI.

In some existing MR scanners, multiple receive channels (with receivers and associated antennas) are used to accelerate or reduce the time needed to perform an MR scan. These approaches are sometimes referred to as ‘MRI parallel imaging.’

Notably, the gradient coils in an MR scanner phase encode (temporally) MR signals, which allows the output MR signals to be distinguished from each other. Moreover, when there are multiple receive channels, there is redundancy in the collected phase-encoded MR signals. In principle, by exploiting the different phase profiles, the redundancy allows some of the phase-encoded MR signals (such as some of the MR scan lines) to be skipped and subsequently reconstructed from the other phase-encoded MR signals, thereby accelerating the MR scan time.

For example, for a 2D space, during an MR scan an RF pulse may be applied, and then the gradient coils in x and y may be opened and an MR scan line in k-space may be read out. These operations (applying an RF pulse and reading out an MR scan line) may then be repeated multiple times for additional MR scan lines (which have different phase encodings) until, e.g., 256 MR scan lines are read out. By using, e.g., 32 receive channels and skipping the measurement of some of these MR scan lines, the MR scan time can be reduced by, e.g., a factor of 2 or 3×.

Note, however, that the reduction in the MR scan time is not a linear function of the number of receive channels. This is because in many MRI parallel imaging techniques additional information is needed to reconstruct the skipped MR scan lines. Consequently, the reduction in the number of MR scan lines is either less than the number of receive channels or a separate pre-scan is used to acquire the additional information.

Notably, there are two principal classes of existing MRI parallel imaging techniques. A first class of approaches (which is referred to as ‘SENSE’, ‘ASSET’, ‘RAPID’ or ‘SPEEDER’) is image domain based after reconstruction of MR signals from individual RF pickup coils or antennas in receive channels (which are sometimes referred to as ‘coils’). In this approach, the number of dropped or skipped MR scan lines may equal the number of receive channels. However, a separate pre-scan is used to determine the coil sensitivities (or coil sensitivity maps) of the receive channels. This is because the measured MR signal using a given receive channel during an MR scan corresponds to a volume integral of the product of a coil sensitivity for the given receiver channel and the time-dependent magnetization of the sample. Moreover, because the polarized magnetic field received by a coil or antenna in the given receive channel depends on its position and orientation, in general each of the coils or antennas in the receive channels has a different coil sensitivity. By performing a pre-scan, the coils sensitivities can be predetermined. Then, in the image domain, sample properties (such as the spatially varying proton density) can be illustrated or presented.

Thus, in existing MR scanners, the first class of approaches may involve the operations of: generating coil sensitivity maps, acquire partial k-space MR data, reconstruct partial field-of-view images from each coil, and unfold/combine partial field-of-view images using matrix inversion. Note, therefore, that the first class of approaches is recast as a linear problem, and which may, in part, be solved using a Fourier transform and an inverse Fourier transform.

A second class of approaches (which is referred to as ‘GRAPPA’) is k-space based. This class of approaches may not use a pre-scan to determine the coil sensitivities. Instead, extra or additional MR scan lines may be acquired near k equal to zero in k-space. By leveraging the smoothness of these so-called ‘auto-calibration lines’ near k equal to zero, the missing (skipped) MR scan lines may be calculated (e.g., by interpolation using the auto-calibration lines).

Thus, in existing MR scanners, the second class of approaches may involve reconstructing the Fourier plane of an image from the frequency signals of each coil (i.e., reconstruction in the frequency domain). Note, once again, that the second class of approaches is recast as a linear problem, and which may, in part, be solved using a Fourier transform and an inverse Fourier transform.

In addition, there are some other (less common) approaches for MRI parallel imaging. Notably, the coil sensitivities and the sample properties (such as the spatially varying proton density) can be determined concurrently (instead of, e.g., using a pre-scan) in a joint reconstruction. For example, in principle, the coil sensitivities and the spatially varying proton density can be calculated from MR signals by solving a nonlinear inversion or inverse problem. However, this nonlinear optimization problem is typically ill defined (e.g., there is no unique solution because it is underdetermined, with more unknowns than can be specified by the measured MR signals).

One approach to solving the nonlinear optimization problem is to use an assumed regularizer to constrain the optimization. For example, the coil sensitivities may be assumed to be smooth. This constraint may allow solutions to be obtained, but in general the analysis time is often very long. Another approach to solving the nonlinear optimization problem is to assume that the coil sensitivities can be represented as a linear superposition of polynomial functions. However, this assumed expansion is often ill-conditioned. Notably, it can be difficult to solve the nonlinear optimization problem with polynomial functions that are higher order than quadratic.

In embodiments of the disclosed computation techniques, the nonlinear optimization problem may be solved without assuming that the coil sensitivities are smooth, are a linear superposition of polynomial functions, or have any predefined closed-form functional representations. Instead, the coil sensitivities may be solutions to Maxwell's equations (i.e., may satisfy Maxwell's equations and, thus, may not be approximations) in the field-of-view of an MR apparatus at a given external magnetic field strength. In addition to being physically accurate, the resulting coil sensitivities may allow the nonlinear optimization problem to be solved much more rapidly than existing nonlinear optimization approaches. Separately or in conjunction with skipped MR scan lines, this capability may significantly reduce an MR scan time.

Furthermore, because embodiments of the disclosed computation techniques (which is sometimes referred to as ‘Maxwell parallel imaging’) do not involve the use of a pre-scan to determine the coil sensitivities or the measurement of auto-calibration lines, Maxwell parallel imaging may be significantly faster than the first class of approaches and/or the second class of approaches described previously for MRI parallel imaging. For example, the MR scan time with Maxwell parallel imaging may be, e.g., at least 2-4× faster than these existing classes of approaches. Indeed, Maxwell parallel imaging may achieve a theoretical limit for the possible acceleration in the MR scan time for a given set of coils, a field-of-view, an external magnetic field strength (or resolution), and for a 2D or a 3D measurement.

Note that Maxwell parallel imaging may be used to accelerate the MR scan time with qualitative or quantitative MR measurements. Thus, Maxwell parallel imaging may be used with MRI, MR fingerprinting, tensor field mapping and/or another MR measurement technique.

In general, the solutions to Maxwell's equations for the coil sensitivities are circularly polarized magnetic fields. These coil magnetic fields may be generated in offline (i.e., not during an MR scan) using numerical simulations in the field-of-view of an MR apparatus. For example, the coil magnetic fields may be calculated by a distribution of currents (such as dipoles) on a surface surrounding the field-of-view in an MR apparatus. In some embodiments, there may be tens of thousands or more random currents on the surface.

However, because of the low frequency (the precession frequency for a proton in an external magnetic field of 1.5 T is 63.87 MHz) and the near-field condition, the currents on the surface may be similar to each other. Consequently, there may be a set of coil magnetic field basis vectors that encompasses or includes the majority of the energy or power in the different coil magnetic fields. For example, a singular value decomposition or an eigenvalue-decomposition technique may be used on the different numerically simulated coil magnetic fields to determine the set of coil magnetic field basis vectors. Then, a given coil magnetic field (and, thus, a given coil sensitivity) may be a linear superposition of the set of coil magnetic field basis vectors. In some embodiments, the set of coil magnetic field basis vectors may include, e.g., 30 coil magnetic field basis vectors. Note, once again, that the coil magnetic field basis vectors may each be a solution to Maxwell's equations. Alternatively, in some embodiments, the coil magnetic field basis vectors may each be an approximation to a solution to Maxwell's equations (such as within 85, 95 or 99% of a solution to Maxwell's equations).

By using the set of coil magnetic field basis vectors, the nonlinear optimization problem may be physically ‘regularized’ and may be solved in much less time. For example, if no regularization assumption is made, the nonlinear optimization problem for a 2D MR scan with 12 coils and having a 256-bit Fourier transform resolution may involve solving 256²+12·256² unknown parameters. The first term of unknowns corresponding to, e.g., the unknown proton density and the second term of unknowns corresponding to the unknown coil sensitivities. As noted previously, this problem is ill-posed, so there is no unique solution and various approximations or assumptions have been used in some of the existing approaches.

In contrast, in Maxwell parallel imaging, instead of solving for the unknown coil sensitivities, the nonlinear optimization problem is determining the coefficients for the different coils in weighted linear superpositions of the set of coil magnetic field basis vectors. Thus, the nonlinear optimization problem for a 2D MR scan with 12 coils, 30 coil magnetic field basis vectors and having a 256-bit Fourier transform resolution may involve solving 256²+12·30 unknown parameters. Therefore, Maxwell parallel imaging may much more rapidly (than existing approaches) solve, e.g., for the unknown proton density and the unknown coil sensitivities, because instead of solving for the unknown coil sensitivities, Maxwell parallel imaging concurrently calculates the coefficients for the set of coil magnetic field basis vectors and, e.g., the proton densities.

Note that in Maxwell parallel imaging a given coil sensitivity may be represented by or equal to a weighted superposition of the set of coil magnetic field basis vectors (i.e., a linear superposition of the products of the coefficients and the corresponding coil magnetic field basis vectors). Moreover, note that Maxwell parallel imaging may more accurately determine the coil sensitivities because, ultimately, it may involve solving Maxwell's equations for physical solutions (the set of coil magnetic field basis vectors) without assumptions. Furthermore, even though the weighted superposition of the set of coil magnetic field basis vectors may be an approximation to a given coil sensitivity, it may be a more-accurate and a physical representation.

In Maxwell parallel imaging, the nonlinear optimization problem may involve iteratively solving (e.g., minimizing) a data fidelity term (the squared absolute value of the difference of the MR signals minus estimated MR signals) subject to constraints. Note that the data fidelity term may incorporate or include a contribution from the coil sensitivities (such as the weighted superpositions of the set of coil magnetic field basis vectors). Moreover, note that the constraints may include: a structure of the spatial distribution of proton or nuclei density (and, more generally, an MR parameter, such as a nuclei density, a relaxation time, etc.), a total variation in the proton density (or an MR parameter), and/or another appropriate regularizer on the proton density (or an MR parameter). In general, the regularization term(s) on the proton density (or an MR parameter) may correspond to those used in image processing. Consequently, the regularization term(s) on the proton density (or an MR parameter) may avoid an L2 norm or a smoothing criterion.

In some embodiments, the nonlinear optimization problem may be solved using a predefined or pretrained neural network or a predefined or pretrained machine-learning model. In these embodiments, the coil sensitivities may, once again, be represented by the weighted superpositions of the set of coil magnetic field basis vectors.

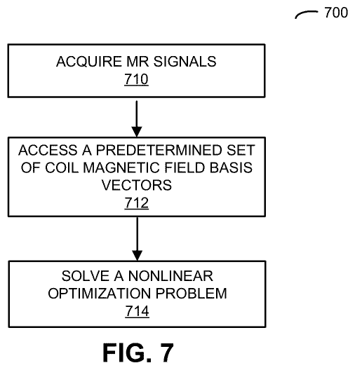

FIG. 7 presents a flow diagram illustrating an example of a method 700 for determining coefficients in a representation of coil sensitivities and MR information associated with a sample. This method may be performed by a system (such as system 100 in FIG. 1), or one or more components in a system (such as source 110, measurement device 114 and/or computer system 116 or, more generally, a computer system that includes one or more computers).

During operation, a computer system may acquire MR signals (operation 710) from or associated with a sample. This may involve having an MR apparatus applying an external magnetic field, a gradient magnetic field, and/or one or more RF pulse sequences, and measuring MR signals using receivers or receive channels. Alternatively or additionally, the computer system may access MR signals stored in memory, which were previously acquired by an MR apparatus or measurement device. Note that the MR apparatus may be located remotely from the computer system or may be proximate to the computer system (such as at a common facility).

Then, the computer system may access (e.g., in memory) a predetermined set of coil magnetic field basis vectors (operation 712), where weighted superpositions of the predetermined set of coil magnetic field basis vectors may represent coil sensitivities of coils in the MR apparatus. For example, a given coil sensitivity may be represented by a linear superposition of products of coefficients and predetermined coil magnetic field basis vectors in the predetermined set of coil magnetic field basis vectors. Note that each of the predetermined coil magnetic field basis vectors may be solutions to Maxwell's equations.

Next, the computer system may solve a nonlinear optimization problem (operation 714) for MR information associated with the sample and the coefficients using the MR signals and the predetermined set of coil magnetic field basis vectors. For example, the computer system may reduce or minimize a term corresponding to a squared absolute value of a difference between the MR signals and estimated MR signals. The term may include or may incorporate a contribution from the coil sensitivities of the coils in the MR apparatus. For example, a given coil sensitivity may be represented by a weighted superpositions of the predetermined set of coil magnetic field basis vectors, where the weights may include coefficients for each of the predetermined coil magnetic field basis vectors. Moreover, the estimated MR signals may correspond to MR information (such as a spatial distribution of one or more MR parameters in voxels, e.g., a proton or nuclei density, a relaxation time, etc.) specified by the MR signals. Furthermore, the nonlinear optimization problem may include one or more constraints on the reduction or minimization of the term, such as one or more constraints corresponding to the spatial distribution of the one or more MR parameters (e.g., a regularizer corresponding to the one or more MR parameters).

In some embodiments, the nonlinear optimization problem is solved iteratively (e.g., until a convergence criterion is achieved). However, in other embodiments, the nonlinear optimization problem is solved using a pretrained neural network or a pretrained machine-learning model that maps the MR signals and the set of coil magnetic field basis vectors to the spatial distribution of the one or more MR parameters (such as in voxels) and the coefficients. Thus, in some embodiments, the nonlinear optimization problem may be solved without iteration.

Moreover, in some embodiments, the spatial distribution of the one or more MR parameters specify a spatial distribution of nuclei density in the sample (e.g., in an image). Thus, in some embodiments, the MR signals may be determined in qualitative measurements, such as MRI or another MR measurement technique. In these embodiments, therefore, the MR apparatus may be an MR scanner.

Alternatively, in some embodiments, the spatial distribution of the one or more MR parameters may correspond to the model parameters discussed previously. Therefore, in some embodiments, the MR signals may be determined in quantitative measurements, such as determining model parameters for voxels in a sample (which is sometimes referred to as ‘tensor field mapping’ or TFM), MR fingerprinting or another quantitative MR measurement technique.

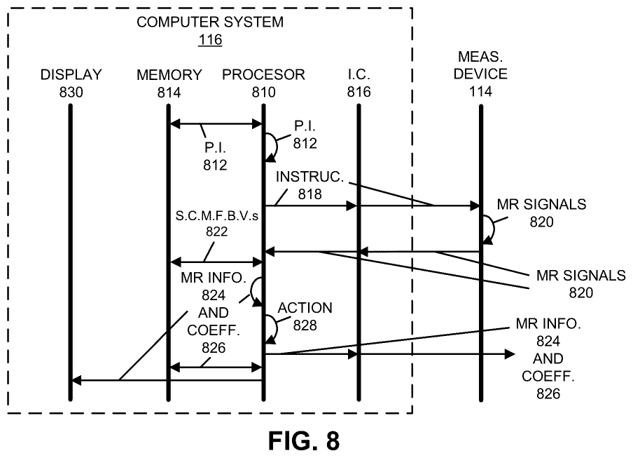

FIG. 8 presents a drawing illustrating an example of communication among components in system 100 (FIG. 1) and measurement device 114. Notably, processor 810 in computer system 116 may execute program instructions (P.I.) 812 stored in memory 814. When processor 810 executes program instructions 812, processor 810 may perform at least some of the operations in the computation techniques.

During the computation techniques, processor 810 may provide instruction 818 to interface circuit (I.C.) 816. In response, interface circuit 816 may provide instruction 818 to measurement device 114 (such as an MR apparatus) to acquire MR signals 820 associated with a sample, which are then provided to computer system 116. Note that in some embodiments measurement device 114 may include a source, such as a source that provides an external magnetic field, a gradient magnetic field and/or an RF pulse sequence to the sample.

After receiving MR signals 820, interface circuit 816 may provide MR signals 820 to processor 810. Then, processor 810 may access in memory 814 a predetermined set of coil magnetic field basis vectors (S.C.M.F.B.V.s) 822, where weighted superpositions of the predetermined set of coil magnetic field basis vectors 822 may represent coil sensitivities of coils in measurement device 114, and a given predetermined coil magnetic field basis vector may be a solution to Maxwell's equations.

Next, processor 810 may solve a nonlinear optimization problem for MR information 824 on a voxel-by-voxel basis in the sample and coefficients 826 in the weighted superpositions using MR signals 820 and the set of predetermined set of coil magnetic field basis vectors 822. Moreover, processor 810 may perform an additional action 828. For example, processor 810 may: provide MR information 824 and/or coefficients 826 to a user or another electronic device via interface circuit 816, store MR information 824 and/or coefficients 826 in memory 814, and/or may present MR information 824 and/or coefficients 826 on a display 830.

In some embodiments, the computation techniques address the problem of MRI reconstruction using multiple MR coils and under-sampled k-space measurements. By solving this problem, the computation techniques may significantly reduce the MR acquisition or scan time, but without compromising the quality of the restored or reconstructed image. This problem is known as ‘parallel imaging’ or MRI parallel imaging.

Because of the limited or reduced number of k-space measurements and the presence of noise, the problem that the computation techniques solves is ill-posed. This means that a unique solution does not exist and, in order to obtain a physically meaningful solution, additional prior knowledge about the properties of the underlying weighted proton-density (WPD) (which is sometimes referred to as the proton density or the nuclei density in the previous discussion) may need to be exploited. Furthermore, another challenge with parallel imaging is that, in addition to the WPD, which is the quantity for which an accurate estimate is desired, the MR coil sensitivities are also unknown.

In order to address this problem, the computation techniques or the Maxwell parallel imaging technique may solve a bilinear problem with respect to the WPD and the coil sensitivities using an iterative Gauss-Newton regularized technique. For example, the computation techniques may include an explicit regularizer on the WPD and an implicit regularizer on the coil sensitivities.

In some embodiments, the regularizer on the WPD can be of quadratic form and involve as a regularization operator: an identity operator, a gradient, a Hessian, a Laplacian or a non-smooth convex regularizer (such as the total variation or the structure total variation. In the case of a quadratic regularizer, because the data fidelity term is also quadratic, an iterative solution may be obtained by solving the augmented Gauss-Newton normal equations. For example, the augmented Gauss-Newton normal equations may be solved by using a conjugate gradient technique. Alternatively, when the regularizer on the WPD is a non-smooth convex functional, then the solution in each Gauss-Newton iteration may be obtained by employing an accelerated proximal gradient technique (such as FISTA).

Moreover, the implicit regularization of the coil sensitivities may be different from existing approaches. Notably, the implicit regularization of the coil sensitivities may enforce that the resulting coil sensitivities (which are essentially the circularly polarized magnetic fields that the coils receive) be smooth. In the implicit regularization of the coil sensitivities, a stronger, physics-based constraint may be imposed. More specifically, a complete (up to a numerical accuracy of, e.g., 85, 95 or 99%) basis of the circularly polarized magnetic fields may be generated. This basis may be supported in the filed-of-view of an MR scanner (or, more generally, an MR apparatus) for a given set of MR coils. For example, the basis may be determined using a randomized singular value decomposition of a matrix that maps the circularly polarized magnetic fields within the field-of-view from a set of tens of thousands or more dipole sources on a surface that encloses the field-of-view and is located close to the given MR coils. The calculation of the magnetic fields by these current sources may involve the use of a full-wave electromagnetic solver that uses of a state-of-the-art volume integral equation technique.

Consequently, in the resulting nonlinear optimization problem, the coefficients of this basis may be determined, instead of the actual coil sensitivities or magnetic fields. This approach may guarantee that the coil sensitivities are not only smooth, but that they satisfy, by construction, Maxwell equations, which is a much stronger constraint (and much closer to reality). Moreover, because of the smoothness of the coil sensitivities, only a small number of the members of this basis may be needed for high-fidelity coil sensitivity estimation. This capability may translate into orders of magnitude fewer parameters in the associated nonlinear optimization problem. Furthermore, the Maxwell parallel imaging technique may be applicable to an arbitrary (i.e., any) magnetic field strength of an MR scanner or an MR apparatus (e.g., from a few milliTesla to 11 Tesla or stronger external magnetic field strengths) without modification.

Thus, the Maxwell parallel imaging technique may provide an estimate of the WPD and an accurate estimate of the coil sensitivities. In order to further enhance the quality of the WPD image or results, in some embodiments, the WPD image may be denoised by solving a constrained optimization problem. Notably, a solution that minimizes the total variation or the structure total variation under the constraint that the norm of the difference of the input and the solution is less than or equal to a quantity that is proportional to the standard deviation of the noise. Note that the standard deviation may be computed directly from the WPD that was estimated previously in the Maxwell parallel imaging technique.

Alternatively, the estimated coil sensitivities, which were determined previously in the Maxwell parallel imaging technique, may be used to cast the original nonlinear problem into a linear one. This linear problem may still be ill-posed, because of the under-sampling of the k-space. Then, a final estimate of the WPD image may be obtained as the solution of a constrained convex optimization problem. Notably, the improved estimate of the WPD image may correspond to a minimizer of the total variation or the structure total variation subject to multiple constraints, whose number may equal to the number of MR coil measurements. Each of the constraints may enforce that the norm of the difference of the coil measurement and the corresponding observation or estimation model, which involves the solution, is less than or equal to a quantity proportional to the standard deviation of the noise effecting the specific coil measurements. These operations may provide a parameter-free denoising technique.

We now describe embodiments of the computing techniques for dynamically determining related content. FIG. 9 presents a flow diagram illustrating an example of a method 900 for dynamically determining related content, which may be performed by computer system 116 or computer system 136 (FIG. 1).

During operation, the computer system may receive information specifying selected content (operation 910). In response, the computer system may dynamically determine the related content (operation 912) based at least in part on one or more prior expert selections that are associated with the selected content and/or one or more predefined or predetermined relationships with the selected content. Then, the computer system may provide the selected content and the determined related content (operation 914).

For example, the selected content may include an image associated with a non-invasive characterization technique. Notably, the non-invasive characterization technique may include: MRI, computed tomography, x-ray imaging or ultrasound. In some embodiments, the image may correspond to a portion of a human body. Moreover, the related content may include a measurement associated with a different measurement technique than the non-invasive characterization technique.

Furthermore, the selected content may include or may be associated with a biomarker, and the one or more predefined or predetermined relationships may be between the biomarker and one or more additional biomarkers. Additionally, the one or more predefined or predetermined relationships may be based at least in part on: genetics information, chemistry, a behavior, an environmental factor, a disease, a comorbidity and/or a biological pathway. In some embodiments, the one or more predefined or predetermined relationships may be based at least in part on medical literature.

Moreover, the content may be associated with a patient, and the one or more prior expert selections may be by a physician and may be associated with one or more different patients from the patient. Note that the patient and the one or more different patients may share one or more characteristics or attributes. For example, the patient and the one or more different patients may have a disease (such as the patient and the one or more different patients may have been diagnosed as having the disease), a risk of having the disease, and/or may be members of a demographic group or a common population.

Furthermore, providing the selected content and the determined related content (operation 914) may include displaying the selected content and the determined related content on a display. Additionally, providing the determined related content (operation 914) may include providing a user interface with user-interface features (such as one or more virtual icons) corresponding to the determined related content.

In some embodiments of methods 200 (FIG. 2), 700 (FIG. 7) and 900, there may be additional or fewer operations. Furthermore, the order of the operations may be changed, and/or two or more operations may be combined into a single operation.

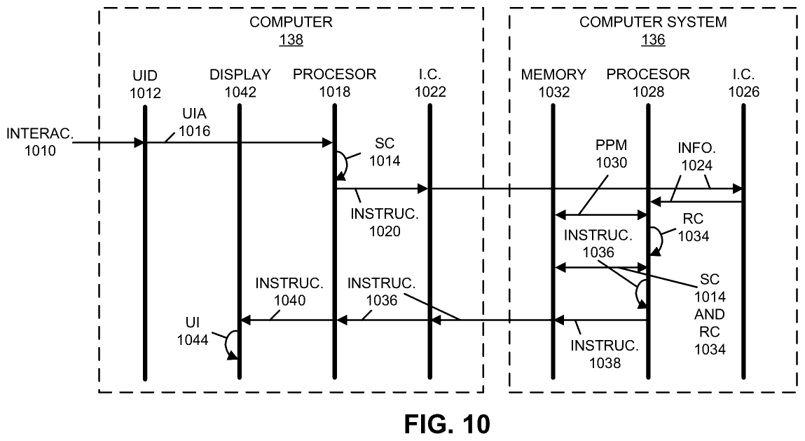

FIG. 10 presents a drawing illustrating an example of communication between computer system 136 and computer 138. Notably, a user of computer system 136 may interact 1010 with a user-interface device (UID) 1012 in computer 138 to specify selected content (SC) 1014. User-interface device 1012 may provide user-interface activity (UIA) 1016 corresponding to interaction 1010 to a processor 1018 in computer 138, which converts user-interface activity 1016 into the specified selected content 1014. Then, processor 1018 may instruct 1020 an interface circuit 1022 in computer 138 to provide information 1024 specifying selected content 1014 to computer system 136.

After receiving information 1024, an interface circuit 1026 in computer system 136 may provide information 1024 to processor 1028 in computer system 136. Based at least in part on selected content 1014, and using a pretrained predictive model (PPM) 1030, which may be access in memory 1032 in computer 138, processor 1028, may dynamically determine related content 1034. Moreover, processor 1028 may access selected content 1014 and related content 1034 in memory 1032, and may generate instructions 1036 for a user interface (UI) 1044 that includes selected content 1014, related content 1034 and/or user-interface features (such as icons or virtual icons, e.g., tabs, radio buttons, check boxes, sliders, etc.) corresponding to selected content 1014 and/or related content 1034.

Next, processor 1028 may provide instructions 1038 to interface circuit 1026, which provides instructions 1036 to computer 138. After receiving instructions 1036, interface circuit 1022 may provide instructions 1036 to processor 1018, which provides instructions 1040 to a display 1042 in computer 138 to display user interface 1044 with selected content 1014, related content 1034 and/or the user-interface features corresponding to selected content 1014 and/or related content 1034.

While FIGS. 3, 8 and 10 illustrate communication between components using unidirectional or bidirectional communication with lines having single arrows or double arrows, in general the communication in a given operation in this figure may involve unidirectional or bidirectional communication. Moreover, while FIGS. 3, 8 and 10 illustrates operations being performed sequentially or at different times, in other embodiments at least some of these operations may, at least in part, be performed concurrently or in parallel.

In the embodiments illustrated in FIGS. 9 and 10, the disclosed computing techniques may include performing statistical analysis of the one or more expert selections. Moreover, alternatively or additionally to the expert selections, one or more of the operations in the computing techniques may be based at least in part on expert behavior. For example, how a healthcare provider (such as a physician) uses or interacts with a user interface (and, more generally, a web page or website) may provide data and/or insights about what information to present. In some embodiments, the behavior may include user selections. However, in other embodiments, the data may include how long a given healthcare provide spent looking a particular content, such as longitudinal measurements of a specific biomarker.

While the present discussion illustrates the computation techniques with noninvasive measurements, in other embodiments the computation techniques may be used in conjunction with invasive measurements.

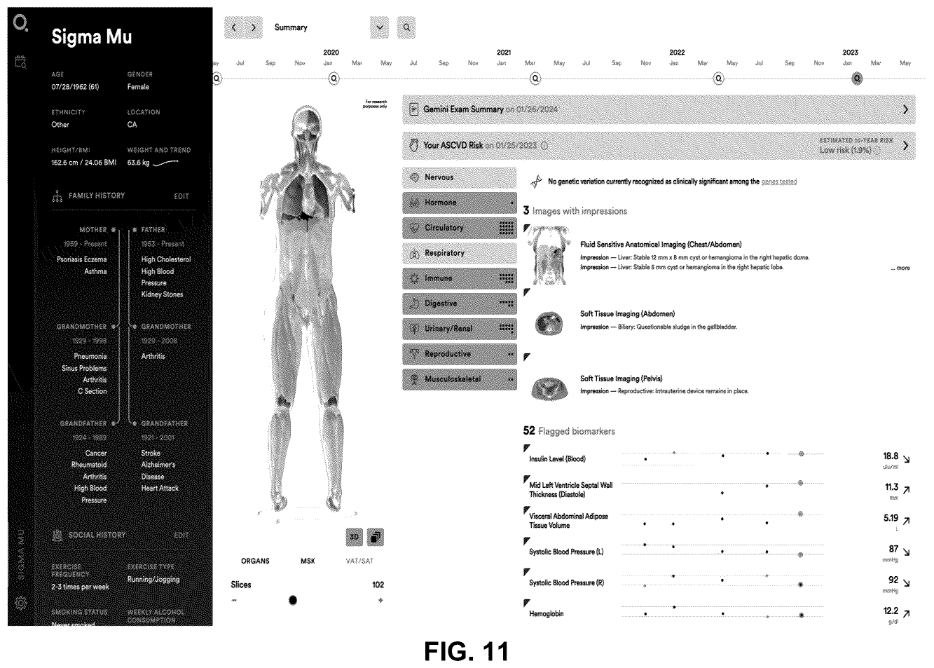

FIG. 11-46 present example of user interfaces. In FIG. 11, a user interface includes tabs for different subsystems in the human body (such as the nervous system, the endocrine or hormone system, the circulatory system, the respiratory system, the immune system, the digestive system, the urinary/renal system, the reproductive system and the musculoskeletal system). These tabs are all currently highlighted, indicating that they have been selected by a user. Adjacent to these tabs, the user interface includes summaries of MRI scans and measurement results for biomarkers (such as triglycerides, total cholesterol, etc.) at different times (as indicated by the circular dots in the longitudinal timelines) for the selected subsystems. Moreover, on the left-hand-side of the user interface, a summary of a medical history for this patient is presented. Furthermore, in the center of the user interface, an MRI image of different portions of the human body is displayed.

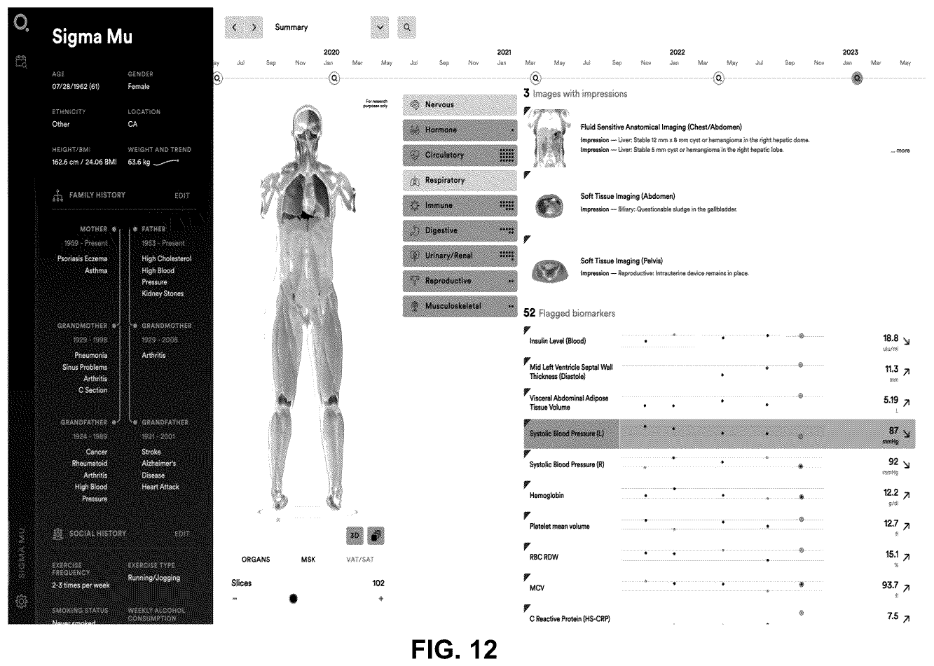

Additionally, in FIG. 12, the user scrolls down the measurement results for the biomarkers and selects eosinophils by clicking on or selecting the corresponding tab. This is an example of selected content in the computing techniques.

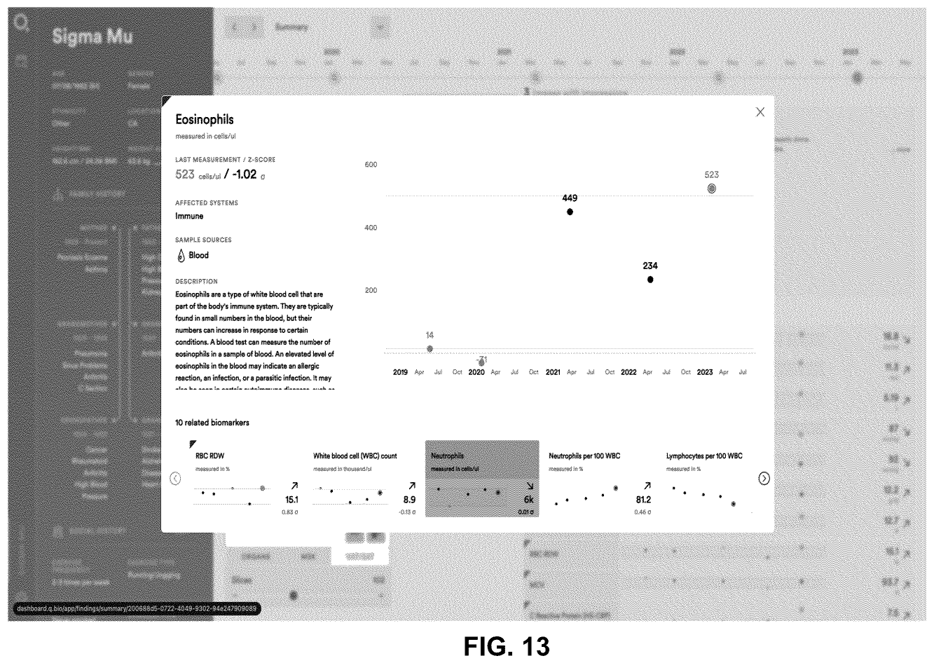

As shown in FIG. 13, in response a longitudinal history of measurement results for the selected content (eosinophils) is presented in an overlaid window in a user interface. In addition, tabs corresponding to longitudinal measurement results for dynamically determined related content is presented. Notably, FIG. 13 includes tabs for related biomarkers, such as: basophils, lymphocytes, monocytes and neutrophils.

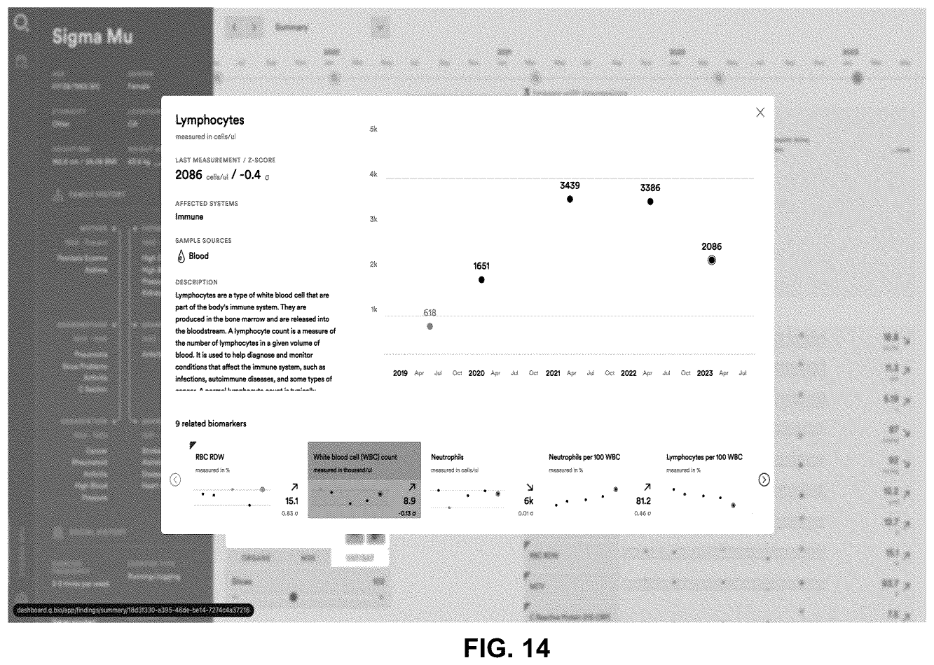

Furthermore, as shown in FIG. 14, by clicking on or selecting the tab for lymphocytes, a longitudinal history of measurement results for lymphocytes is displayed.

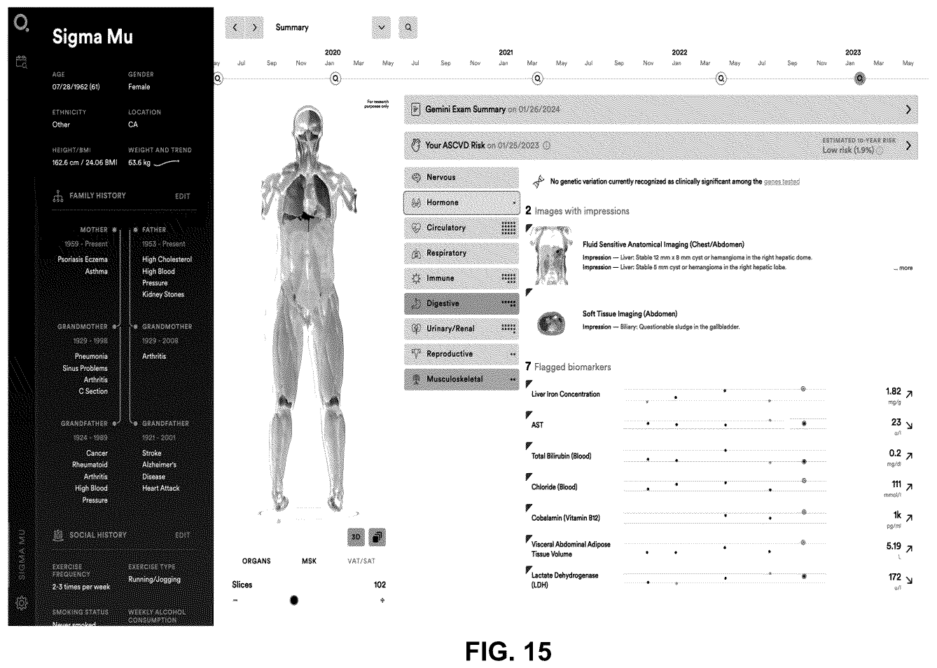

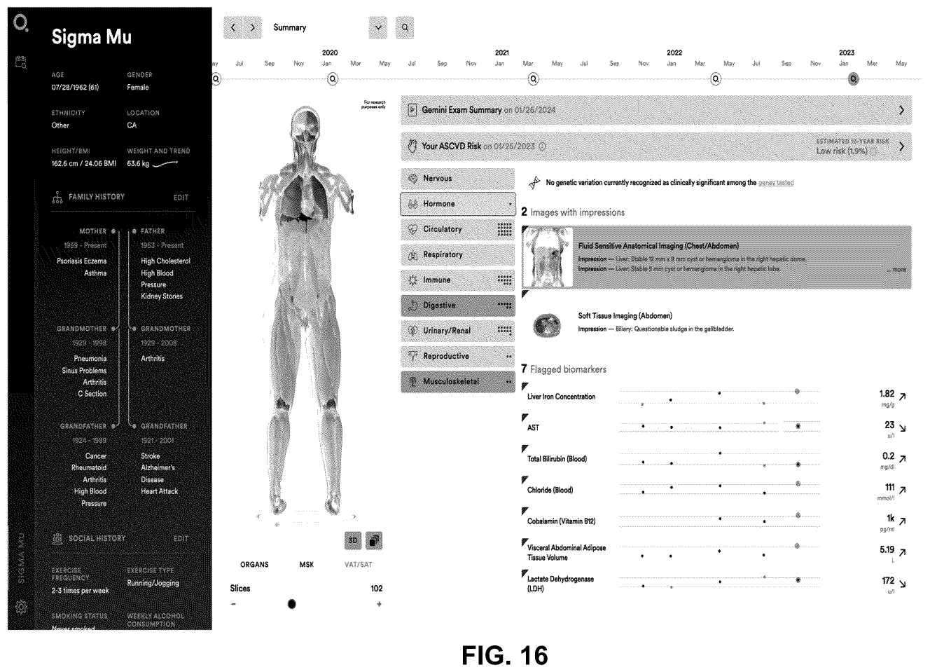

Then, as shown in FIG. 15, by clicking on or selecting the back icon, the overlaid window is removed and a user interface with the summary is presented again. Note that by a clicking on or selecting a given tab, the user can deselect (or select) a corresponding subsystem, thereby removing (or adding) any associated summaries of MRI scans and measurement results for biomarkers that are displayed in the user interface. As shown in FIG. 15, only the nervous system and the musculoskeletal system are currently selected. Additionally, as shown in FIG. 16, the user may select content from the displayed measurement results, e.g., MRI scans of a developmental C3-C4 segmentation/fusion anomaly.

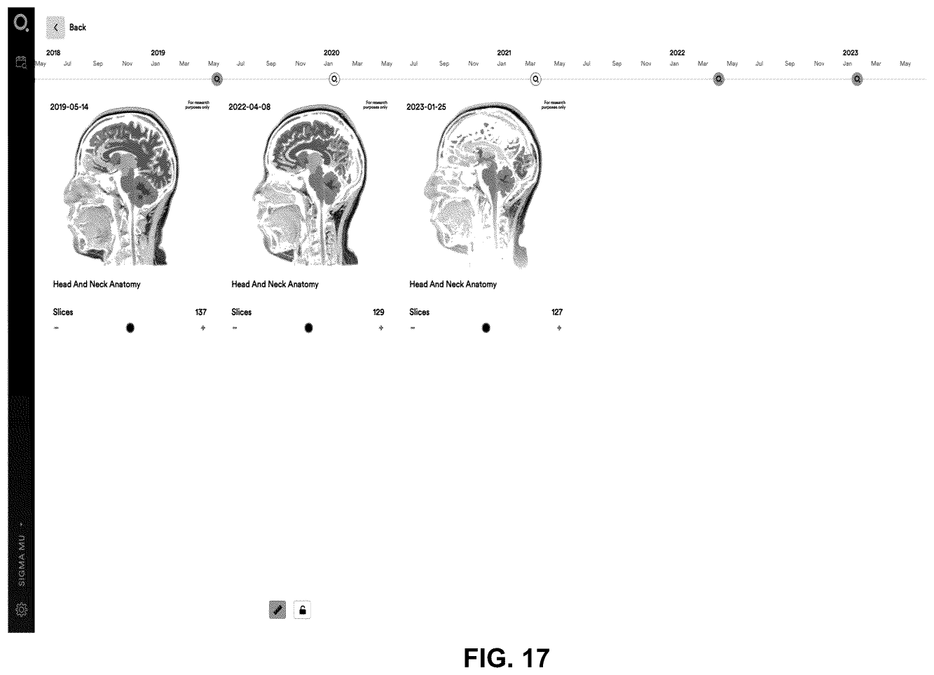

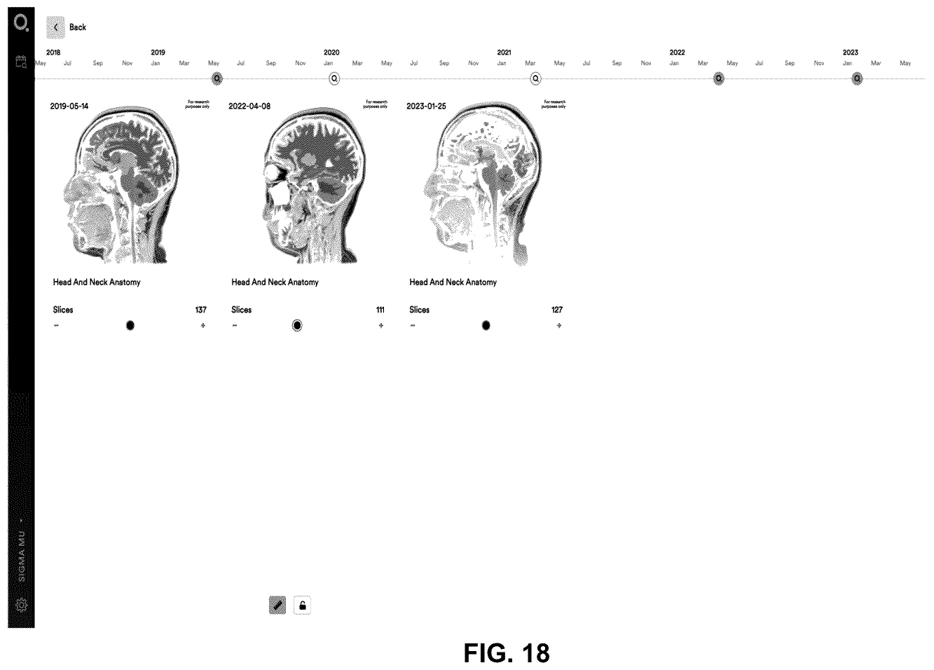

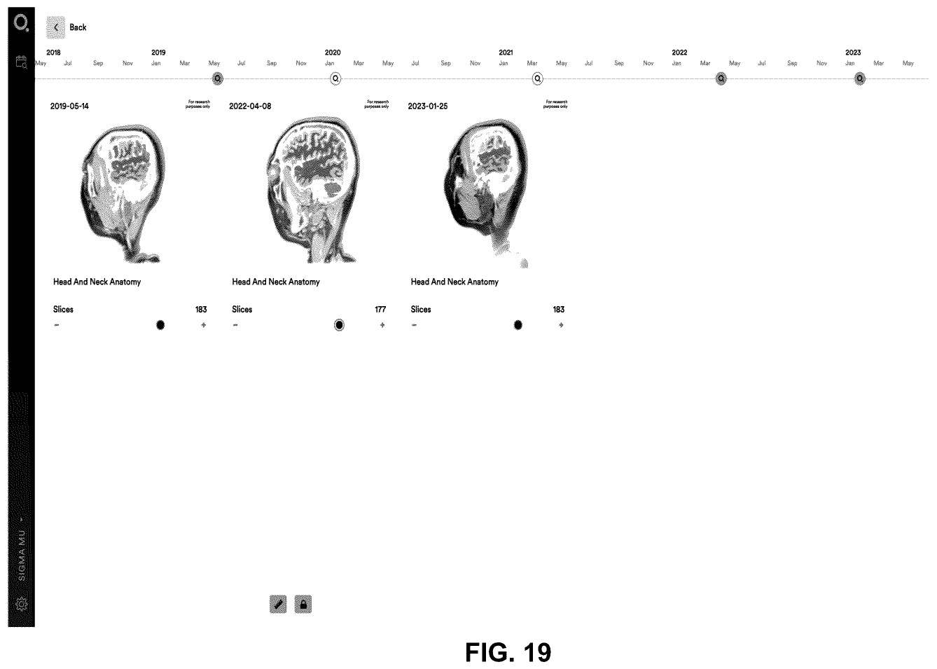

FIG. 17 presents a user interface with a longitudinal series of MRI scans. In this example, there is no related content (such as any related biomarkers). By clicking on or selecting and dragging or moving the slider associated with a given MRI scan (such as the Nov. 17, 2019 MRI scan), the displayed slice in the given MRI scan can be changed. This is shown in FIGS. 18 and 19.

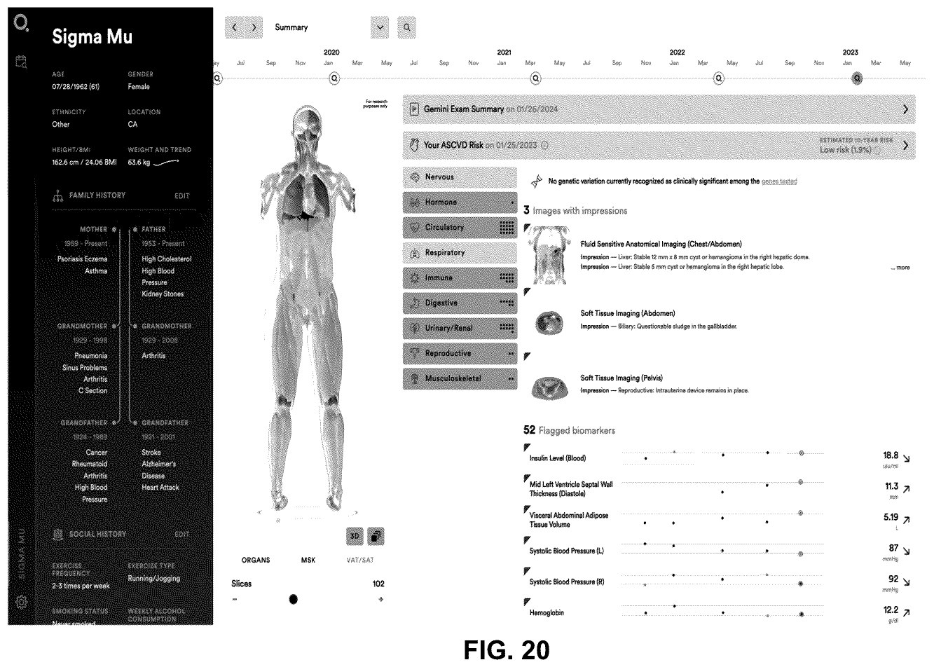

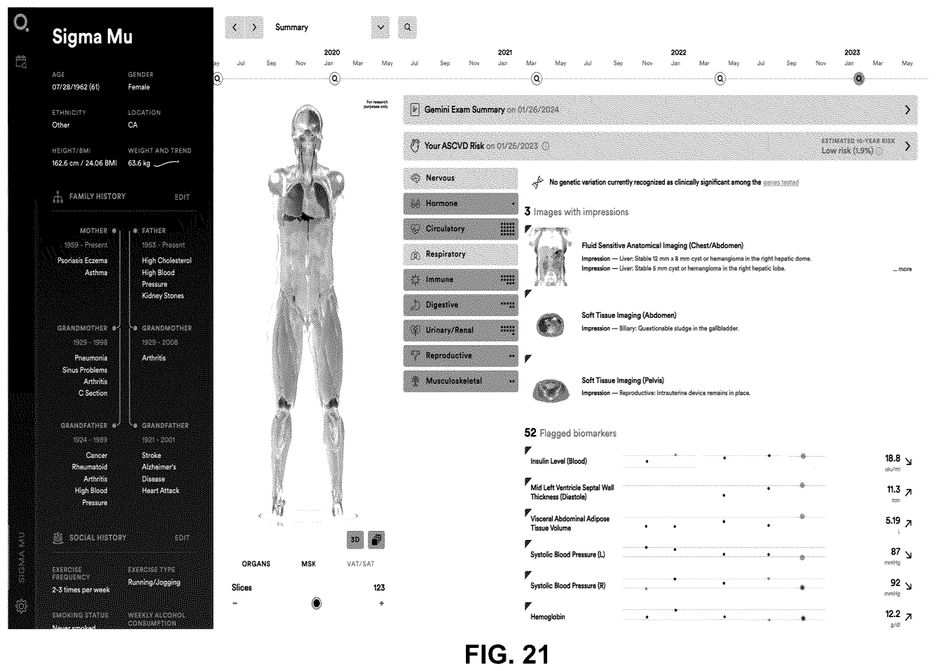

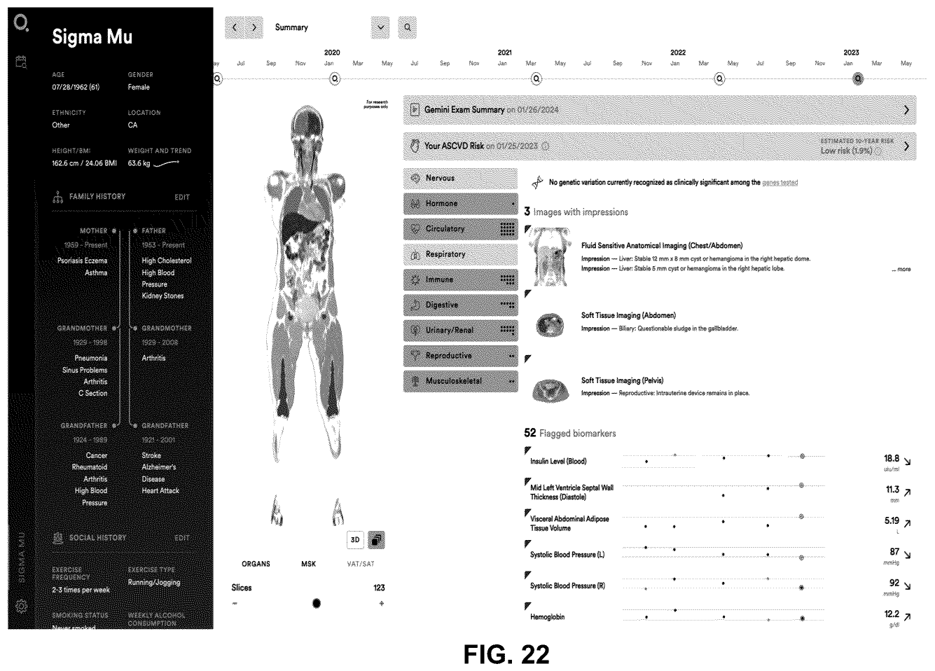

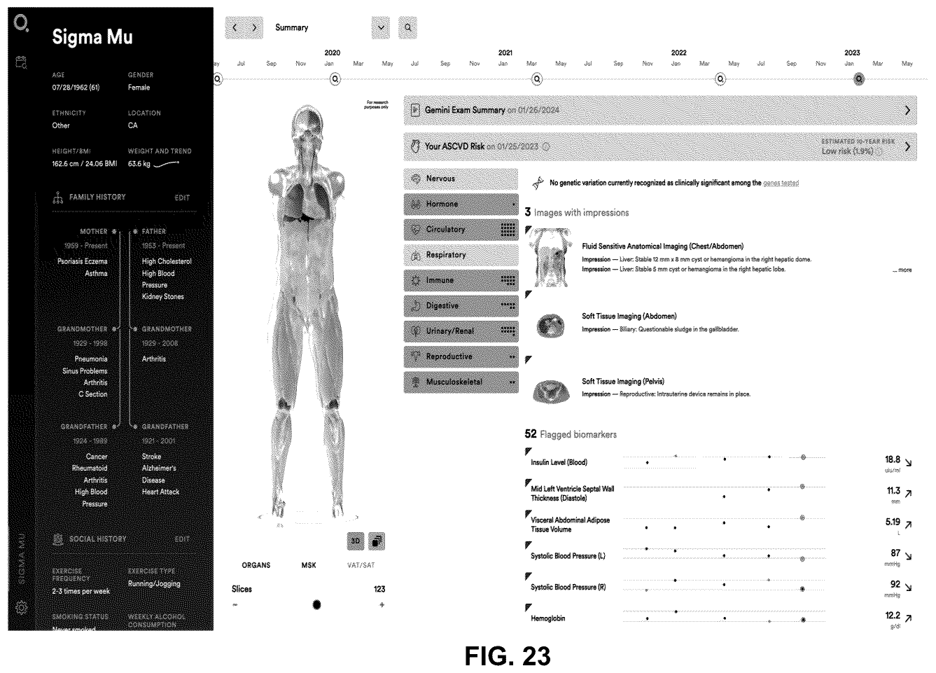

As shown in FIG. 20, by clicking on or selecting the back icon, a user interface with the summary is presented again. Moreover, as shown in FIG. 21, by clicking on or selecting and dragging or moving the slider in the organ tab for the central MRI image, the displayed slice can be changed. Alternatively, as shown in FIG. 22, by clicking on or selecting the visceral adipose tissue/subcutaneous adipose tissue (VAT/SAT) tab, fat is highlighted in the MRI image. Furthermore, as shown in FIG. 23, by clicking on or selecting the MSK tab, the musculoskeletal subsystem is highlighted in the MRI image.

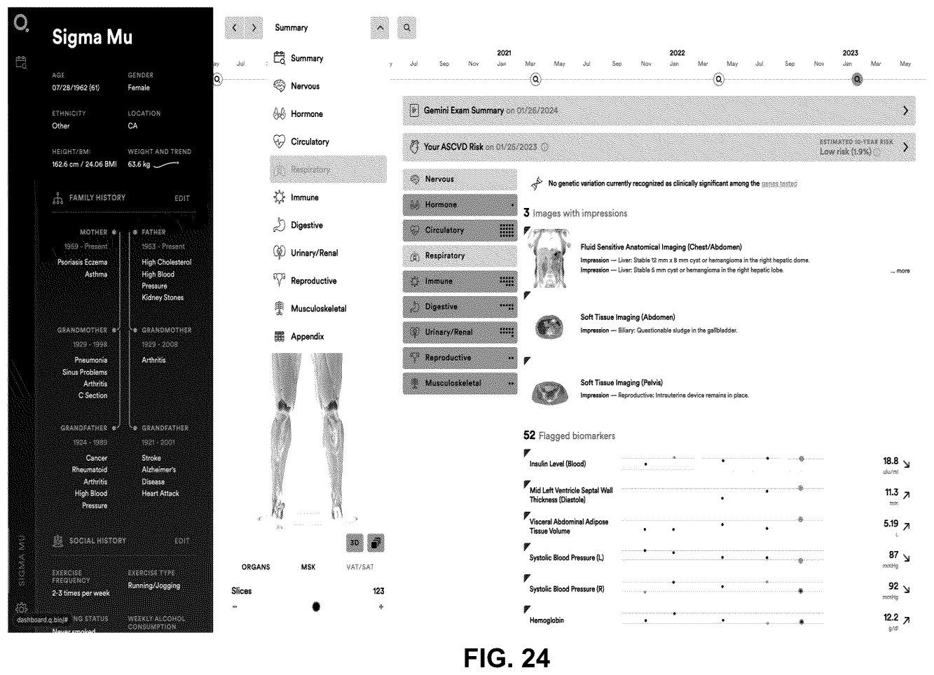

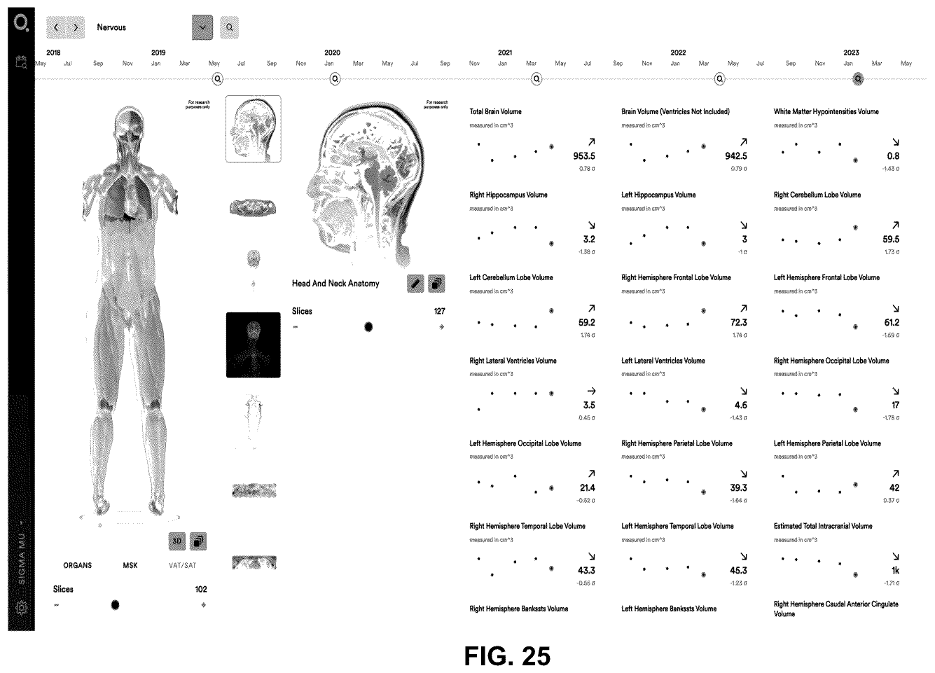

As shown in FIG. 24, the user may also select MRI scans for different subsystems in the body by clicking on or selecting an arrow icon for a dropdown list. For example, as shown in FIG. 25, by selecting the nervous system, a longitudinal history of MRI scans of the nervous system is presented. For a given MRI scan, summary metrics may be displayed.

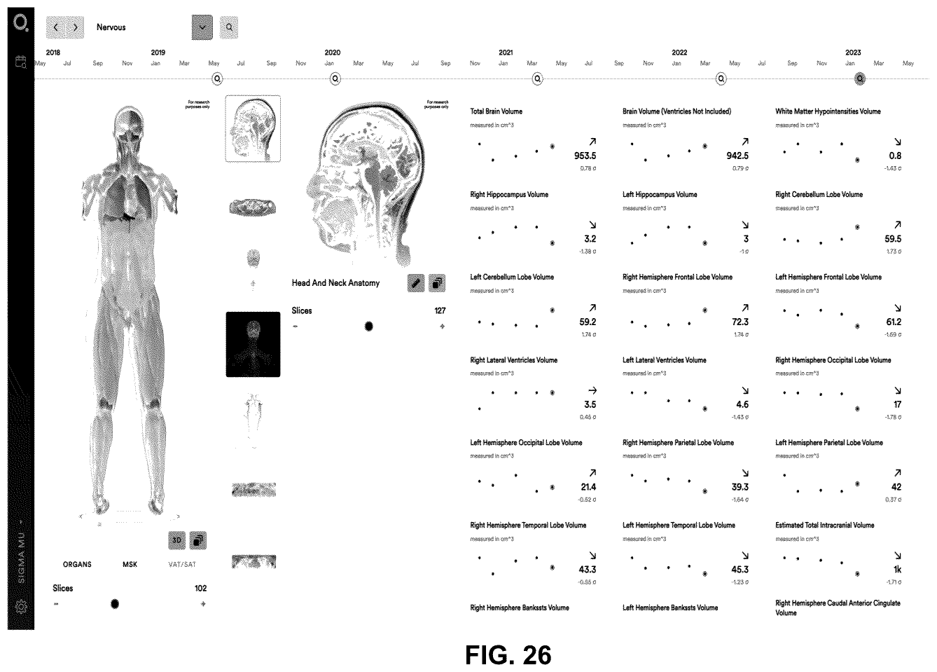

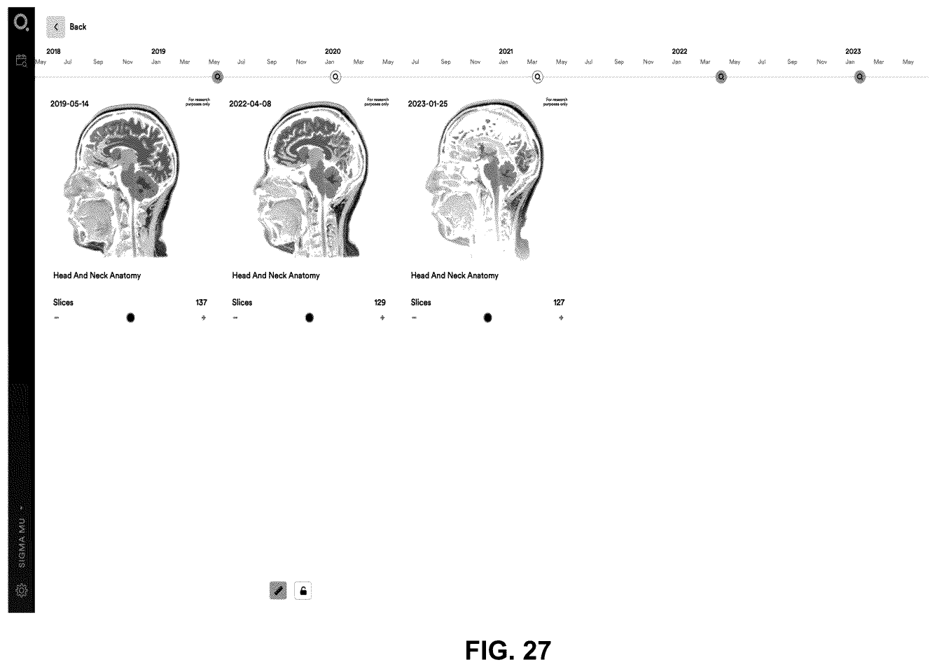

Additionally, as shown in FIG. 26, the user may click on or select a sequence icon. As shown in FIG. 27, a longitudinal series of MRI scans is presented.

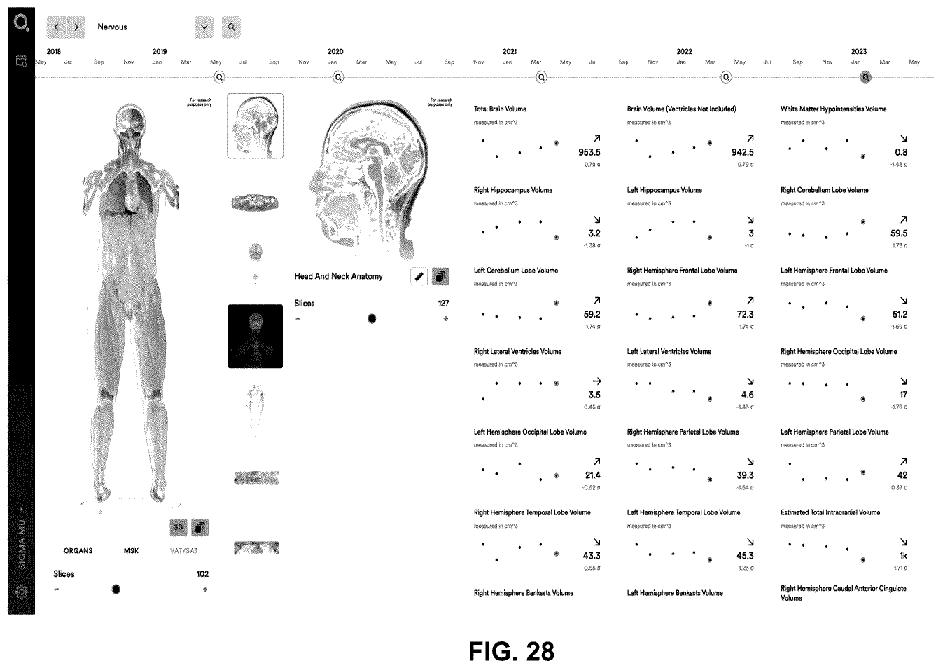

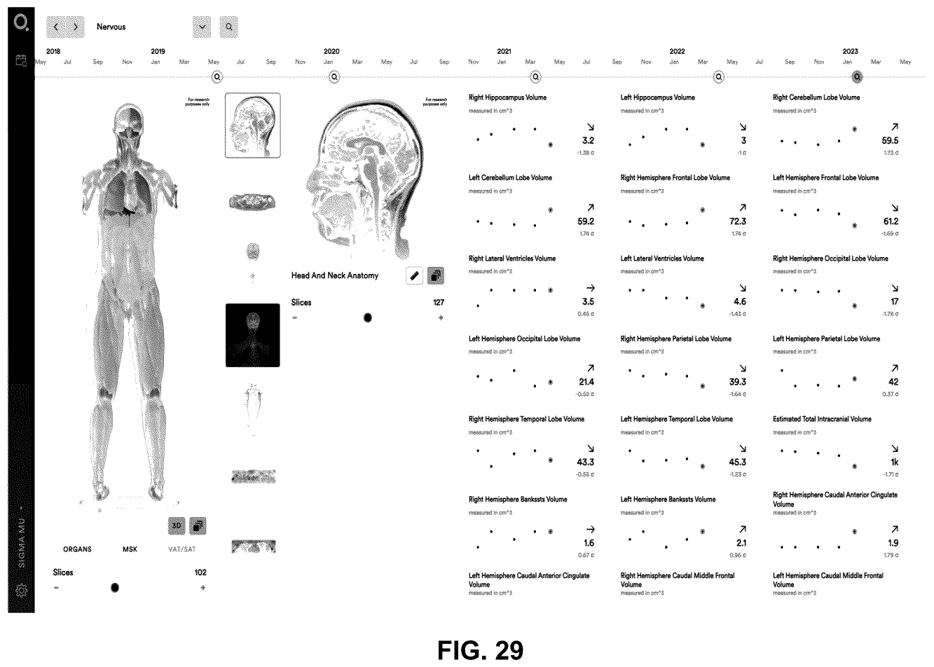

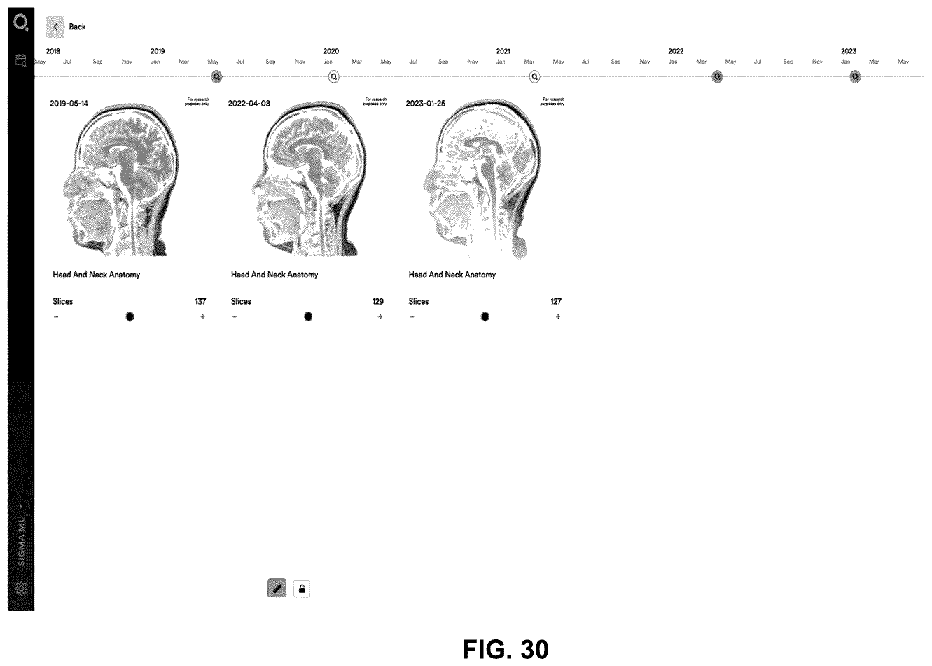

Alternatively, as shown in FIGS. 28 and 29, the user may switch select a greyscale icon or a colorization icon. For example, when the colorization icon is selected, different portions of the central nervous system may be highlighted using associated colors. As shown in FIG. 30, when the user clicks on or selects the sequence icon again, a longitudinal series of MRI scans with the color highlighting (shown as greyscale) is presented.

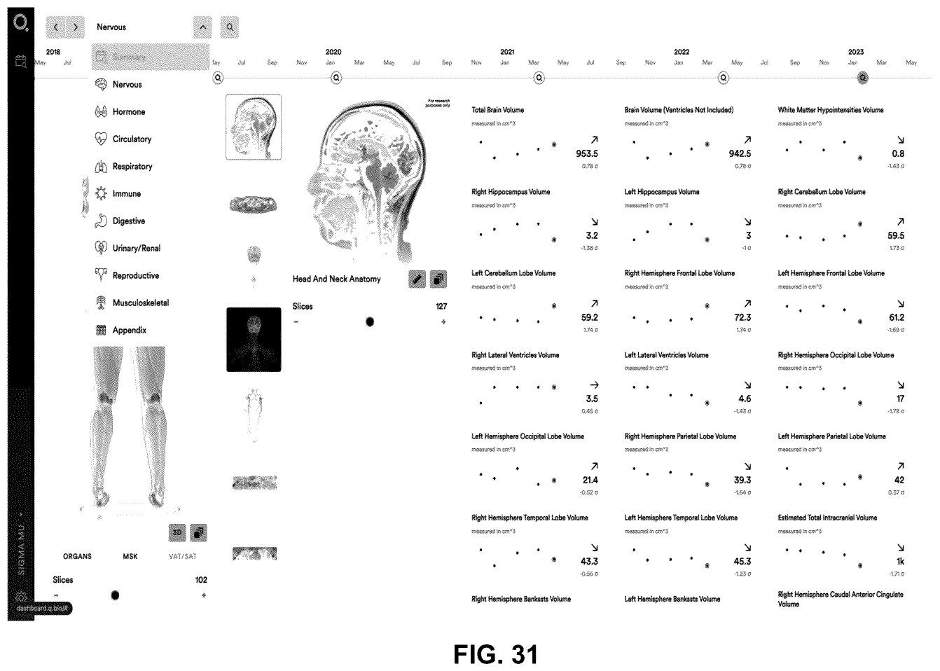

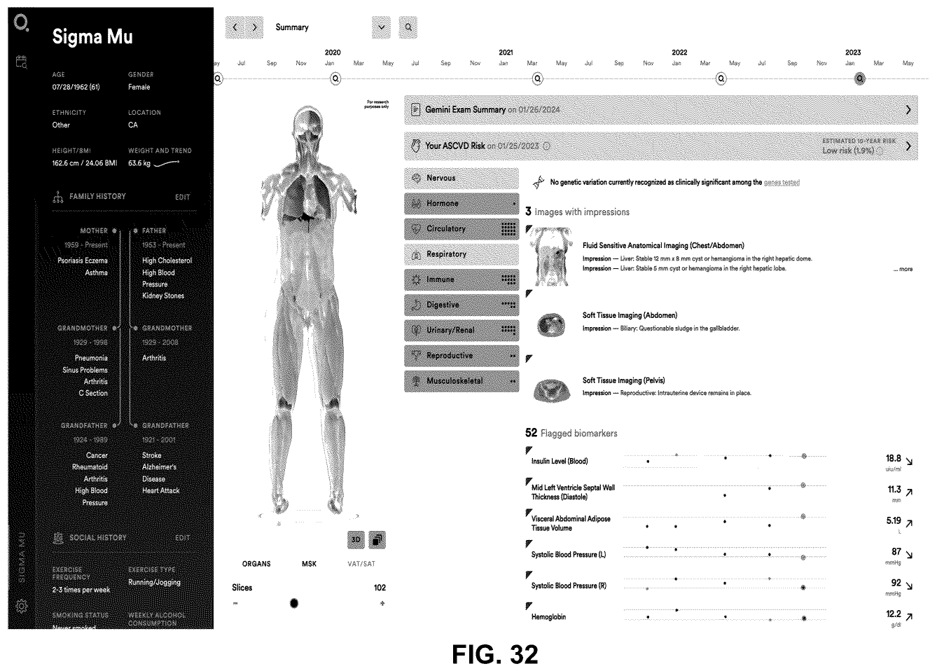

After clicking on or selecting a back icon, as shown in FIG. 31, the user may select MRI scans for different subsystems in the body by clicking on or selecting the arrow icon for the dropdown list. Alternatively, by after clicking on or selecting a back icon again, as shown in FIG. 32 a user interface with the summary is presented.

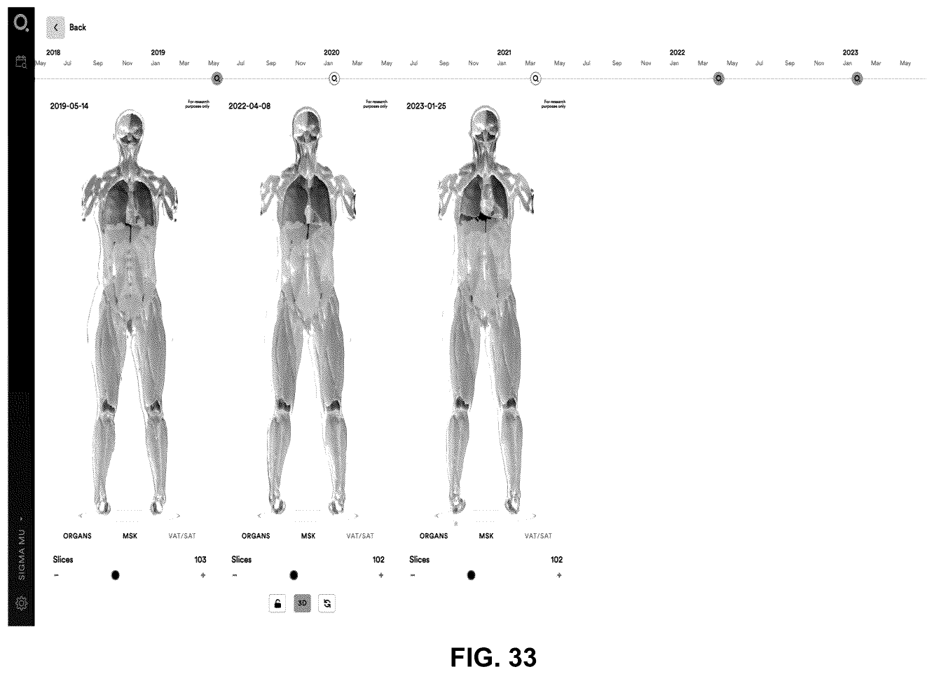

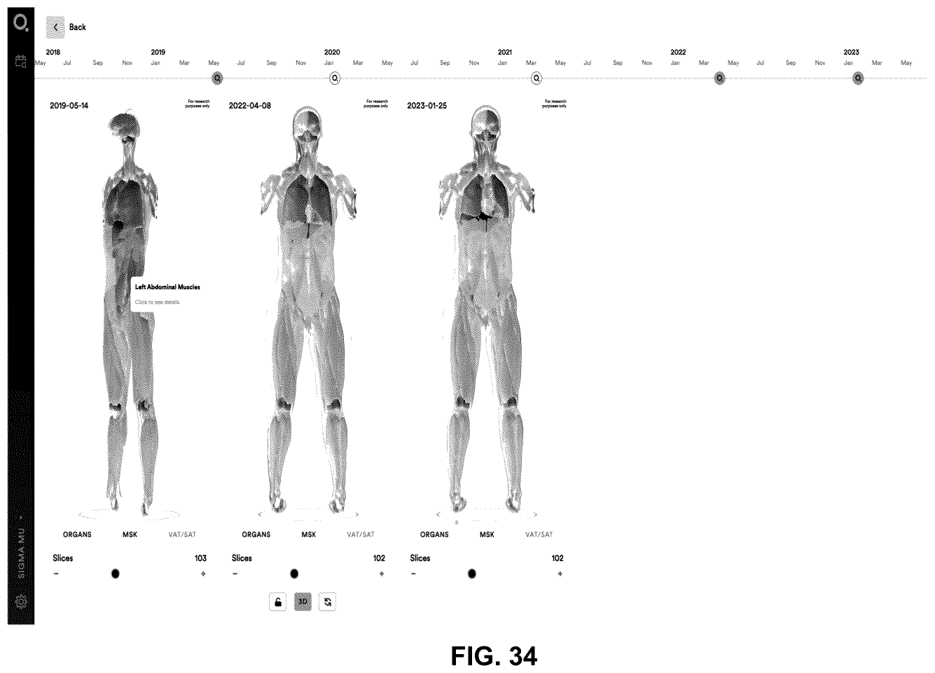

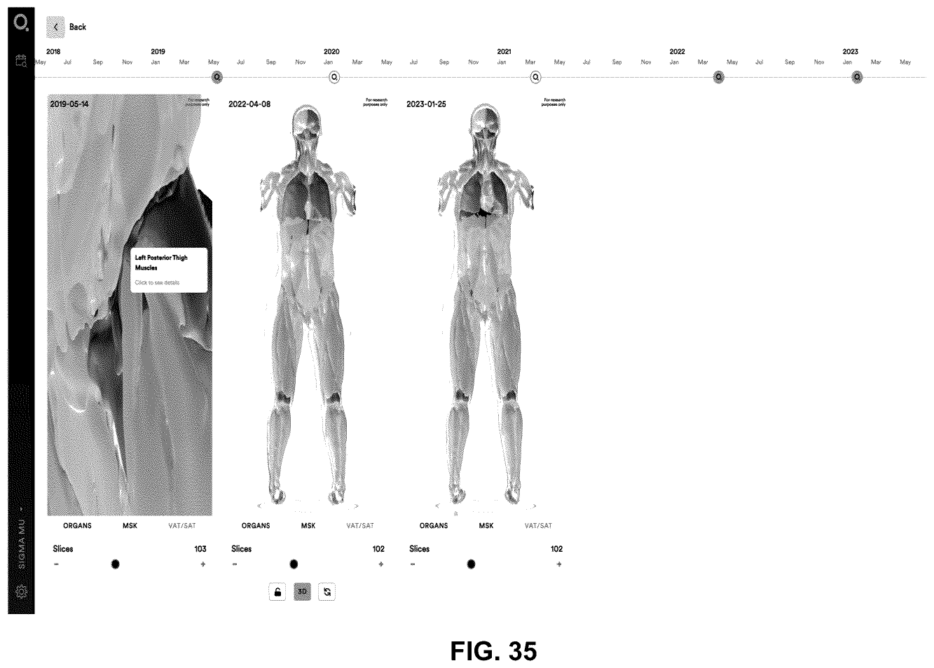

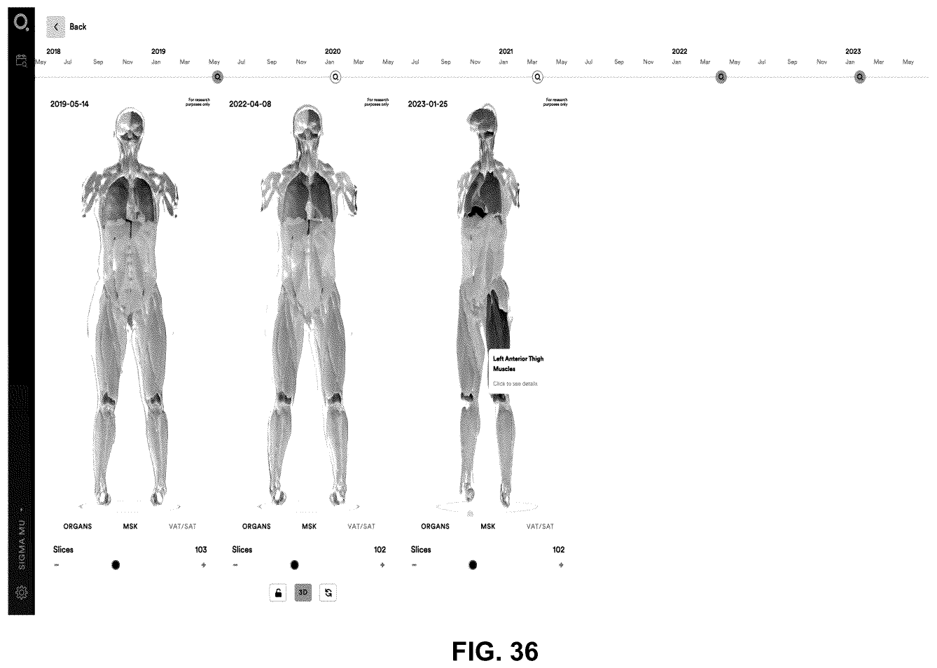

When the user clicks on or selects a sequence icon in the central MRI scan for the organ tab, a user interface in FIG. 33 is displayed. This user interface includes three instances of an MRI image of the human body, which highlight a current selected subsystem (such as the digestive system). As shown in FIG. 34, the user may click on or select the highlighted portion of the human body (such as the fat in the digestive system corresponding to the VAT/SAT tab). Moreover, as shown in FIGS. 35 and 36, by rotating their wrist while clicking on or selecting the highlighted portion of the human body, the user may independently rotate the highlighted portion of the human body in any of the three displayed instances. Alternatively or additionally, by repeatedly clicking on or selecting the highlighted portion of the human body in any of the three displayed instances, the user may independently increase (or decrease) the magnification of any of the three displayed instances.

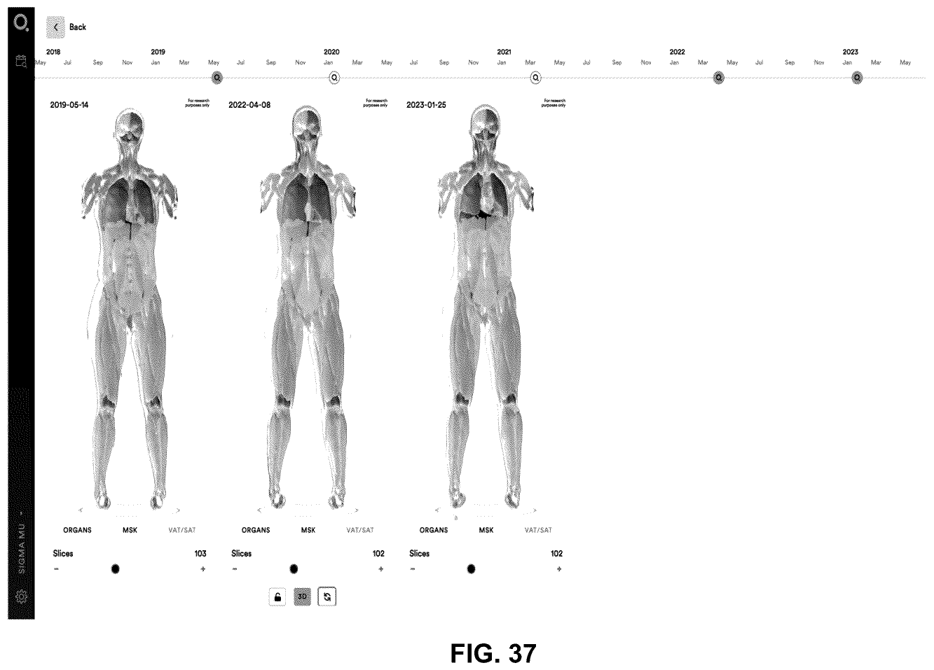

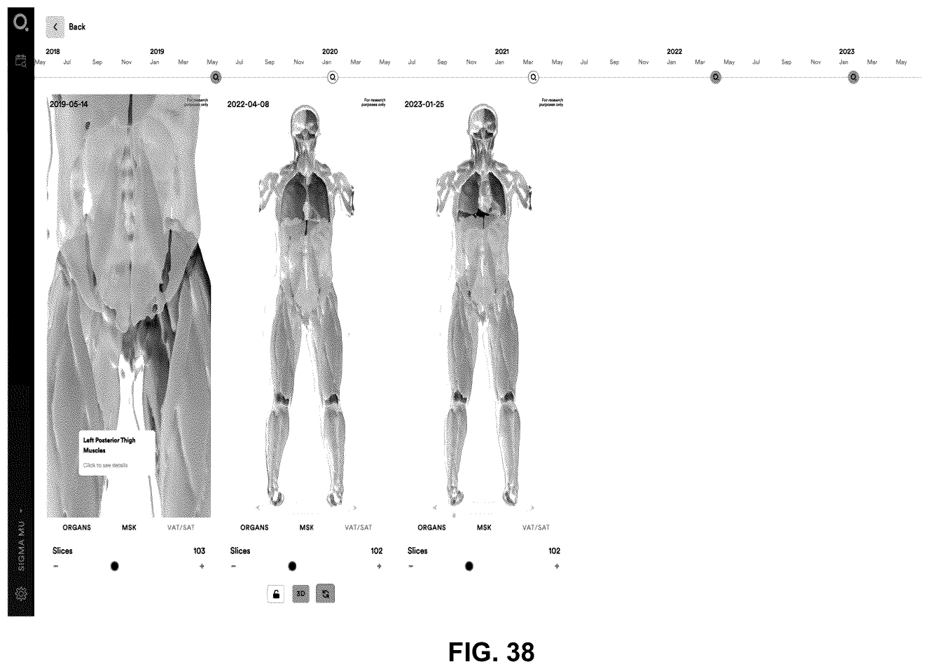

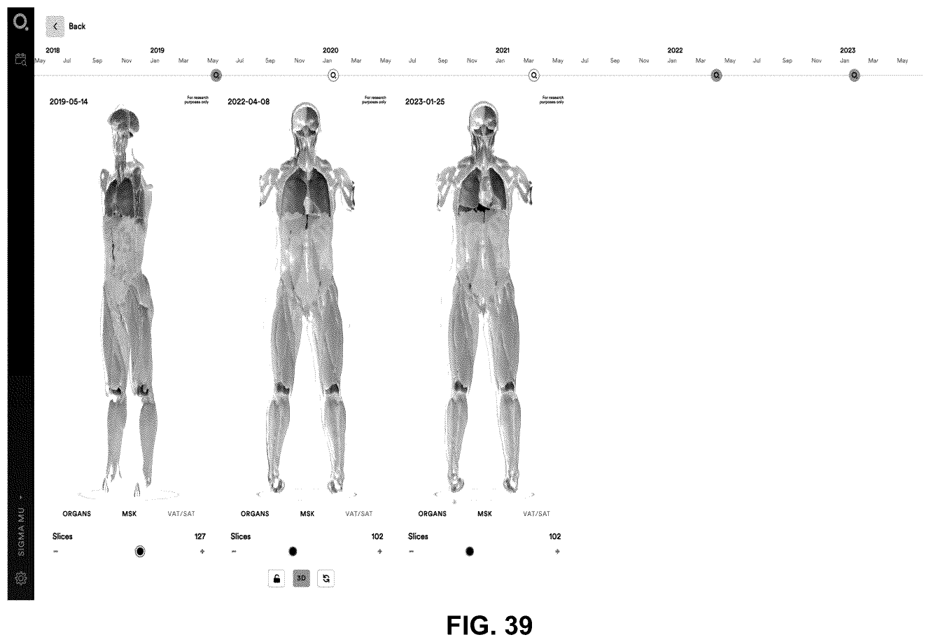

Moreover, as shown in FIG. 37, by clicking on or selecting the organ tab, the three instances of the MRI image of the human body may highlight organs in a current selected subsystem (such as the musculoskeletal system). As shown in FIGS. 38 and 39, the user may independently change the orientation and/or the magnification of any of the three displayed instances.

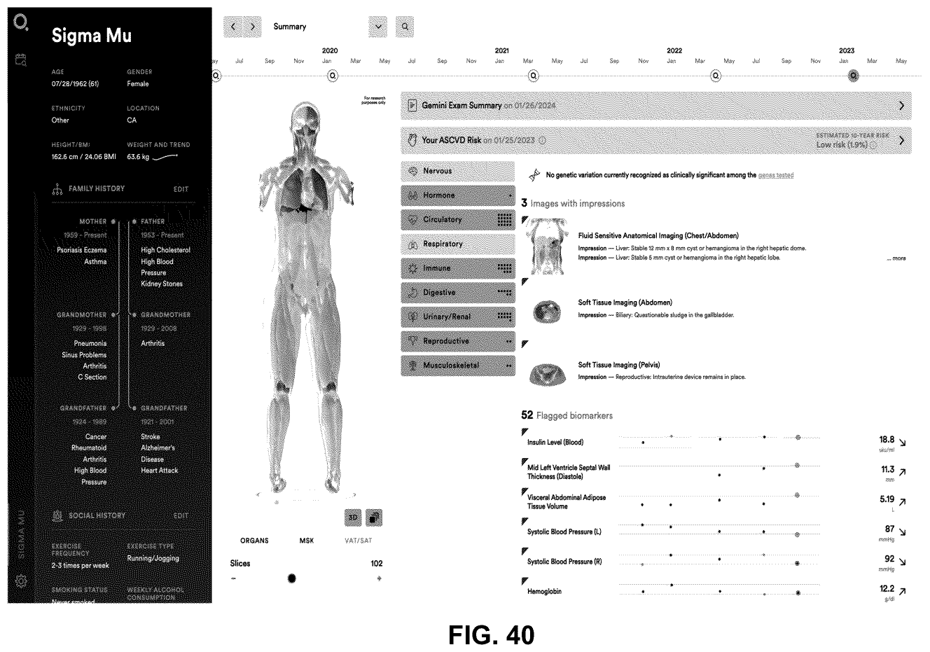

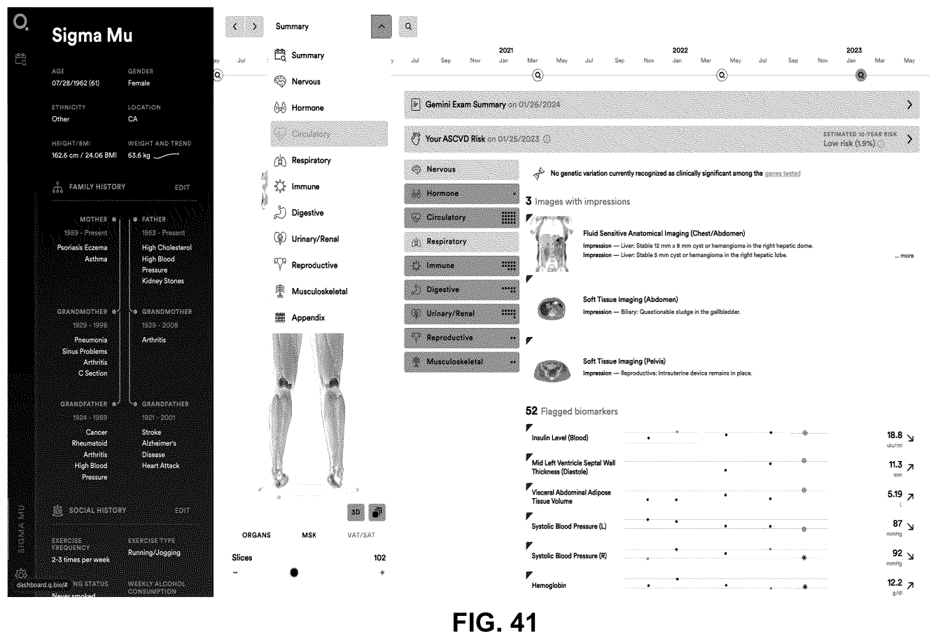

After clicking on or selecting a back icon, as shown in FIG. 40 the user interface with the summary may be presented. Moreover, as shown in FIG. 41, the user may select the circulatory system. This is another example of selected content.

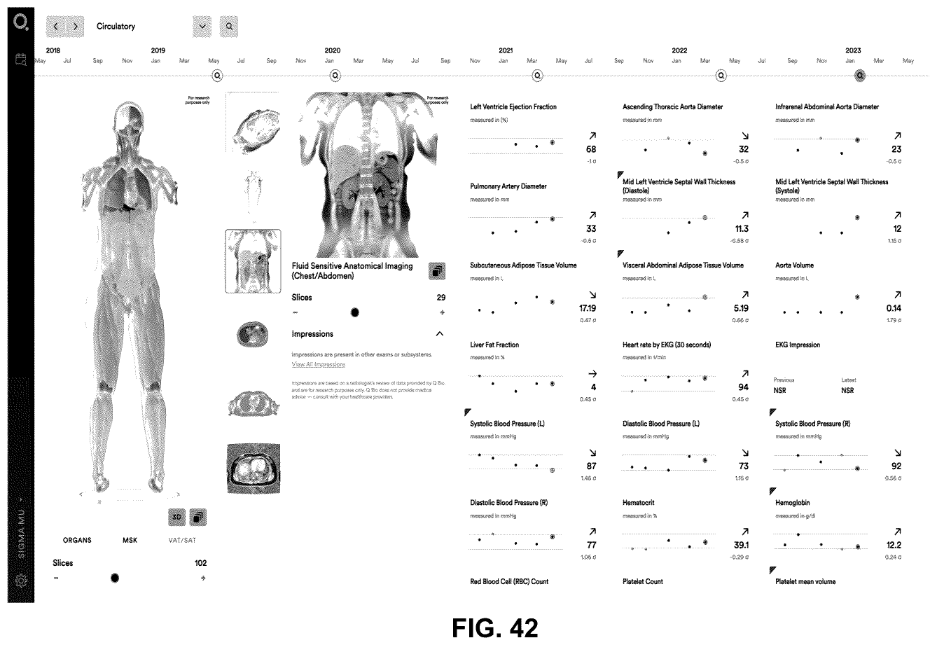

In response, as shown in FIG. 42, a longitudinal history of MRI scans of the circulatory system is presented. In addition, measurement results for related content (such as biomarkers related to the MRI scans) are presented. For example, when a given MRI scan at a timestamp is selected, the related content (such as measurements results for triglycerides) in a time interval that includes the timestamp is displayed.

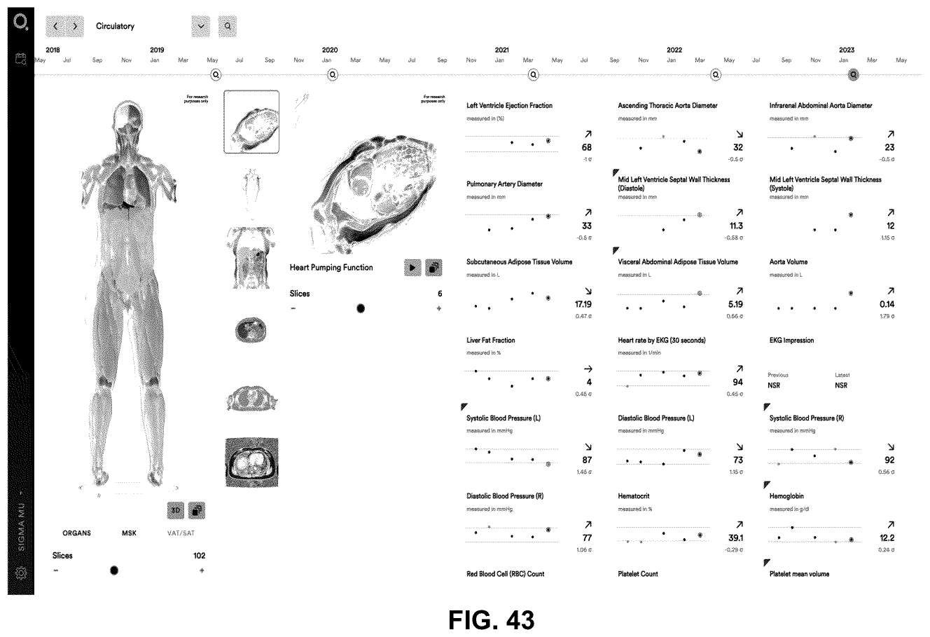

Moreover, as shown in FIG. 43, after selecting a tab corresponding to an MRI scan of a portion of the human body, the user may click on or select a play icon associated with a central MRI image, a video of measured cardiac motion and circulatory flow may be displayed.

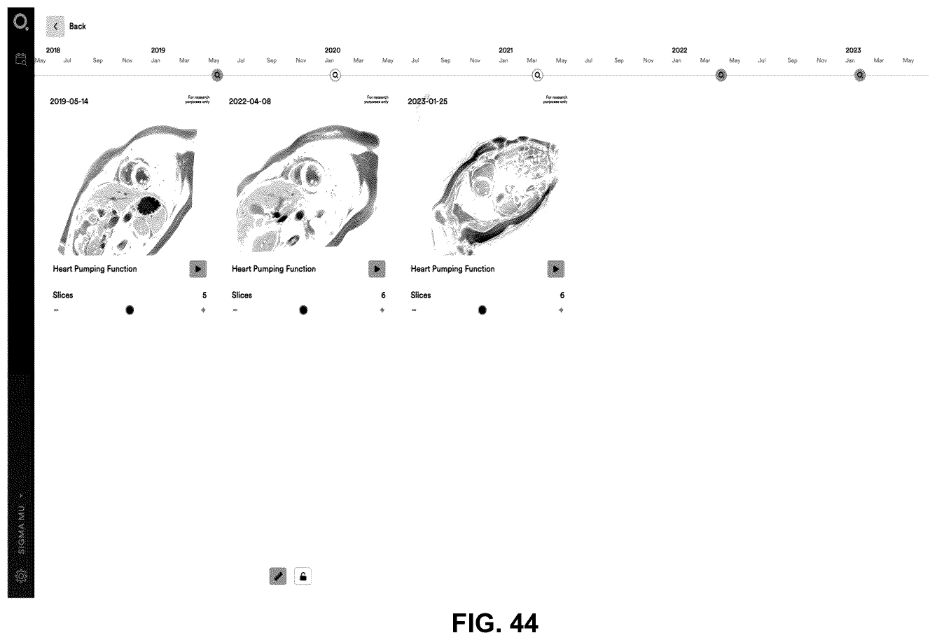

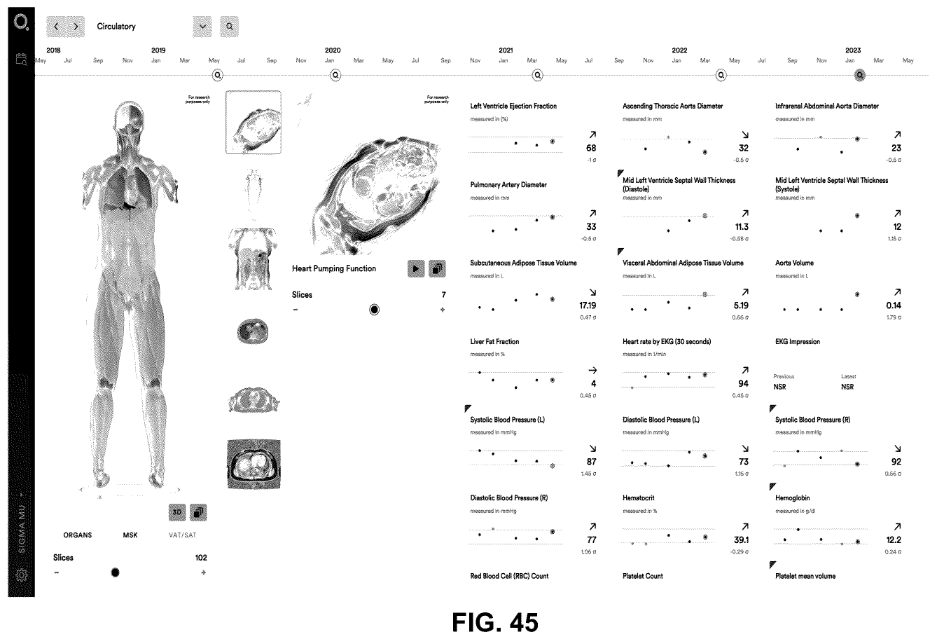

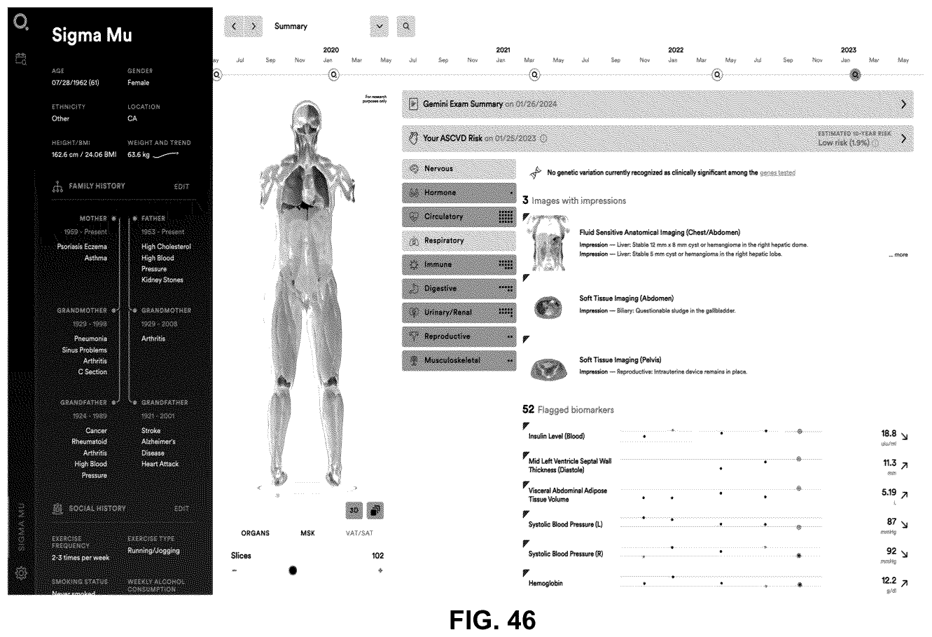

Furthermore, as shown in FIG. 44, when the user clicks on or selects a sequence icon, a longitudinal series of MRI scans is presented. Additionally, when the user clicks on or selects a medical history icon on the left-hand-side of the user interface, as shown in FIG. 45, a summary of the medical history of the patient may be displayed. In some embodiments, when the user clicks on or selects a back icon, as shown in FIG. 46, the user interface with the summary is presented.

In some embodiments, the user interfaces may include fewer or additional user-interface features, a position of a user-interface feature may be changed, two user-interface features may be combined into a single user-interface feature, and/or a single user-interface feature may be divided into multiple user-interface features.

We now describe techniques for a data-driven physical, which may facilitate preventive healthcare. Notably, at a given time t, for a person pi in a population P, there may exist a set of measurements m; in M that can be used to reduce or minimize a total cost of healthcare Cₚ for pᵢ over a time interval [t, dt]. Note that mi may not necessarily be the same for all pᵢ in P (there may be personalization). Moreover, mi may not necessary be the same for a given pᵢ across all time intervals (there may be changes in time). Furthermore, as cost(mᵢ) decreases, there may exist mᵢ′ such that Cₚ′ is less than or equal to Cₚ-cost(mᵢ) (it may not be zero sum).

A trend driving the data-driven physical is that the price of measuring one thing and the price of measuring everything are converging asymptotically. Multi-scale biological digitization is occurring. This may include: meso-scale measurements (e.g., intra-cellular), micro-scale measurements (inter-cellular), macro-scale measurements (intra-organ) and whole-body measurements (inter-organ). For example, meso-scale measurements may include: genomics, epigenomics, transciptomics, proteomics and/or metabolomics. Moreover, micro-scale measurements may include: cytomics and/or microbiomics/metagenomics. Furthermore, macro-scale measurements may include: non-invasive radiomics (e.g., one or more non-invasive characterization techniques) and/or kinesiomics (wearables).

In may eventually get to a point where measuring everything about everyone will make sense. Until then, there may be a tradeoff in terms of the cost of a set of measurements on an individual and the return on investment of those measurements in terms of the quality of care and the cost of care for that individual. In order to show a return on investment of a set of longitudinal measurements on an individual, objective metrics may be used, such as: a total cost of care in a time interval; a cost adjusted for improved outcomes; and/or a total cost of care per year of life adjusted for quality of life.

In some embodiments, it may be advantageous to work backwards from pathologies that have properties, including: may be common in an asymptomatic population and may pose existential risk; improved outcomes may be a function of how early the pathology is caught; the cost of treatment may be inversely proportional to the stage a pathology is caught; and/or there may exist two or more biomarkers that can be gathered non-invasively with clinically accepted sensitivity and specificity to diagnosing a pathology. By building a data structure of known clinical biomarkers, their cost, their sensitivity and specificity to disease, the cost of care depending on stage of detection, and the outcome depending on the stage of detection, the problem may be solved, e.g., by performing Monte Carlo simulations of populations based on the prevalence of various pathologies in a given population based at least in part on: the data in the data structure, existing medical knowledge, and simulating the measurements of a set of biomarkers at a given frequency to determine a likelihood of catching pathologies in the population at various stages. This may provide understanding about how the frequency of measurements and the set of measurements affect the total cost of care and outcomes of a population.

There may be two parameters that have a major impact on these simulations that are not well understood, which may need to be defined or which may require reasonable assumptions about. The first is how the sensitivity/specificity of a specific biomarker improves when it is measured as a time series rather than a point measurement. Another way to think of this is what is the improvement in the signal-to-noise ratio by averaging many measurements. Borrowing from information theory, it may be reasonable to argue that the signal-to-noise ration improves proportional to the square root of N, where N is the number of measurements. This implies that the more something is measured in an individual, the better we will be able to detect anomalous change.

The second parameter is the information orthogonality between biomarker A and B that both have some accepted clinical sensitivity and specificity for a given pathology. It can be argued this is a dominant unknown and, thus, may be important to understand. Because it is unknown, we may simulated IV_{P}(A,B) (information orthogonality of A and B for predicting pathology P) for many different values to determine what this value would have to be for a given protocol to break even from a return-on-investment perspective. Then, we can assess if the breakeven point is based on a believable information orthogonality.

As an example, consider prostate cancer. Assume that the prostate-specific antigen (PSA) as a point measurement has a sensitivity/specificity of X/Y and a specific prostate MRI protocol as having sensitivity X′/Y′. If these two biomarkers had completely independent information (total information orthogonality), then the best we could hope is the sum of their predictive power. This is not very useful because not only is it highly unlikely they are completely uncorrelated, but if they are used in medicine today, they probably have a predictive power that's better than flipping a coin (which is 50%), in which case their combined predictive power would be over 100% (which is not possible). Consequently, the information orthogonality really is a constant we can define as XX′/YY′. This specifies how much extra predictive power we get by combining these two biomarkers, as opposed to looking at them independently. Note that, if these biomarkers each have independent sensitivity/specificity, it is highly likely that each has some independent predictive power. The computational model may just have to answer how much better combining them would have to be in order to justify the cost of the measurement on some time interval or frequency.

This example is, in some ways, an oversimplification, because many biomarkers have sensitivity/specificity to more than one pathology, but the cost to measure a single biomarker is only incurred once at a point in time. Therefore, this is a non-linear problem that may not be reduced to analyzing single sets of biomarkers for single diseases. This means that to fully understand the return on investment you may need to simulate the sensitivity/specificity for the entire set of measurements to all targeted pathologies.

Note that existing diagnostic techniques may be broken, which may further emphasize information value and orthogonality. Notably, existing techniques for developing diagnostics may be fundamentally broken because they do not accept the reality that they ignore: human health has a long tail-distribution; and human health may be based on non-stationary patterns.

The current diagnostic approach is, at a given time, to classify an individual as healthy or not based on comparison to a population. In principle, improved performance may be achieved by segmenting the population into subpopulations and assigning an individual to a particular sub-population for comparison.

However, a comparison to a population reference may be inherently flawed, because what is normal/healthy for one person may be significantly different than for another and because these metrics even averaged over the population may change over time. This is because of changes in our behavior, technology, diet, environment, etc. For example, the average height and weight of a baby has changed dramatically over the last 50 years. Consequently, if these variables are used as inputs into a risk score, then based on averages from 50-years ago, most present-day babies are outliers.

Clinical studies often try to isolate the information value of a single biomarker for predicting a single disease in order to develop a population reference. In principle, this may be useful for understanding a single biomarker and how it relates to disease. However, in practice, if you are trying to build predictive models for diseases with improved accuracy, the aforementioned approach may not only be a gross oversimplification of complicated disease processes, it may handicap the predictive models. It would be impossible to perform sufficient clinical studies to measure the information orthogonality for all biomarkers and all pathologies. If there are N biomarkers and P pathologies, it may require (N/2)·P clinical studies to determine the overlapping information value of combining any two biomarkers for predicting a disease. To make matters worse, the cardinality of N and P may be changing. This implies that in order to develop robust multivariate diagnostics, we may need to make it cheap enough to measure and monitor many things in the general population and to continuously be able to update the predictive models as we gather more information and the population changes. This approach (which is sometimes referred to as a ‘data-driven physical’) may address the shortcomings in existing approaches.

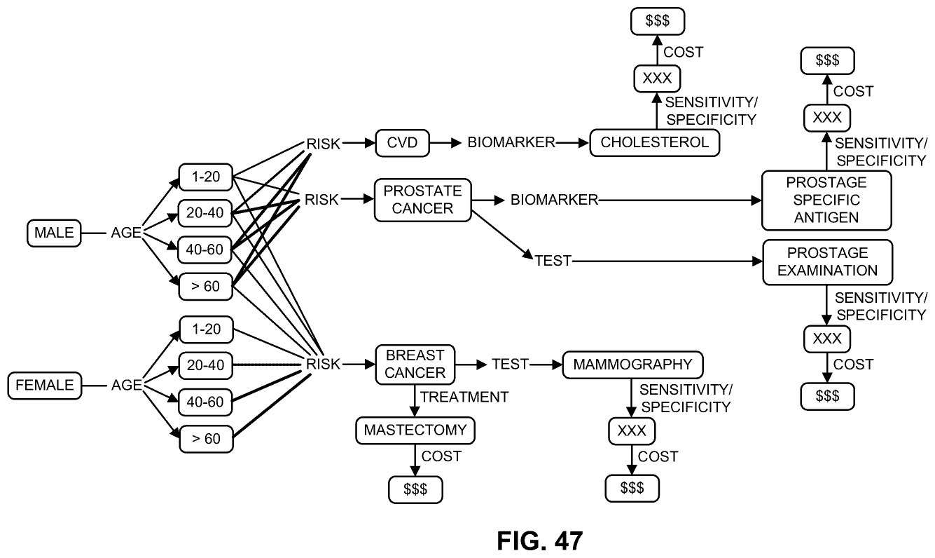

In some embodiments, a custom diagnostic tool may allow modeling of different protocols on different populations. This diagnostic tool may provide a current protocol and may allow the integration of additional biomarkers or changes to the protocol to be assessed. The diagnostic tool may be used to personalize the set of measurements on an individual basis. Note that the diagnostic tool may also allow us to understand what the coverage of a given protocol is in terms of known diseases it is sensitive to, etc. FIG. 47 presents a drawing illustrating an example of a protocol diagnostic tool.

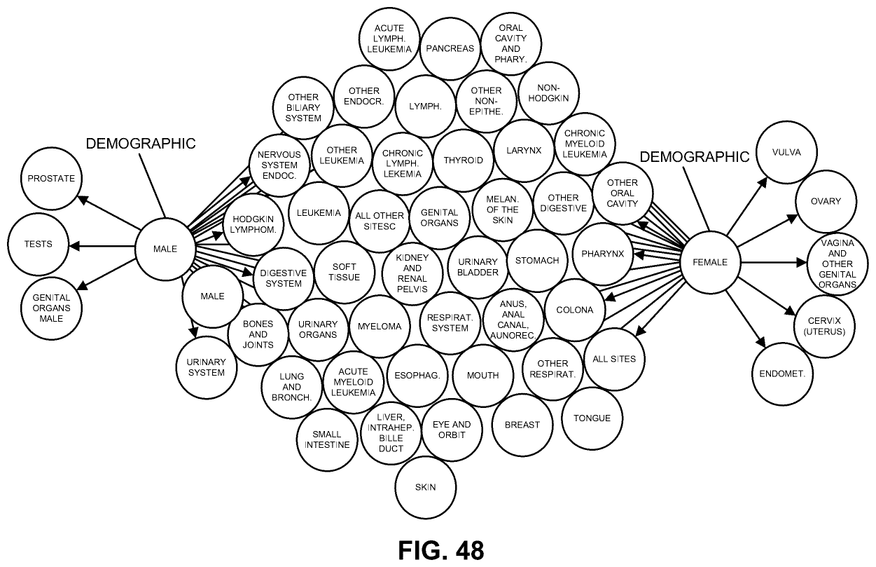

Moreover, there may be a relationship between demographics and pathology. Notably, incidence (e.g., cases and deaths) may connect a demographic to a pathology. FIG. 48 presents a drawing illustrating an example of a graph relating demographics (male/female) with pathologies of different organs or organ systems. This graph structure may reveal demographic-specific pathologies. The arrows in FIG. 48 illustrate incidences of different pathologies. Note that FIG. 48 may summarize 220,800 cases and 27,540 deaths.

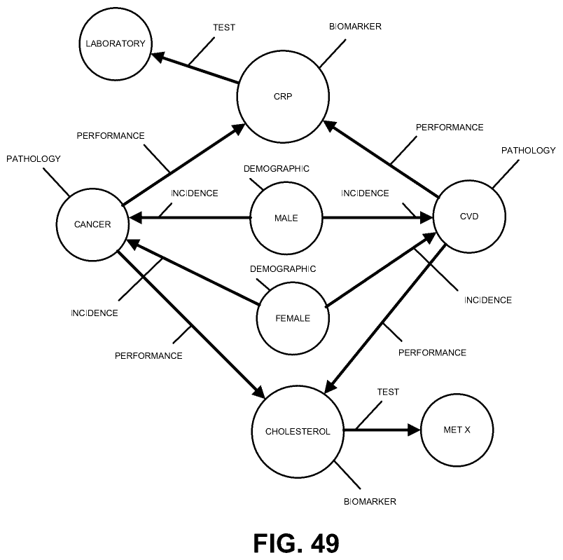

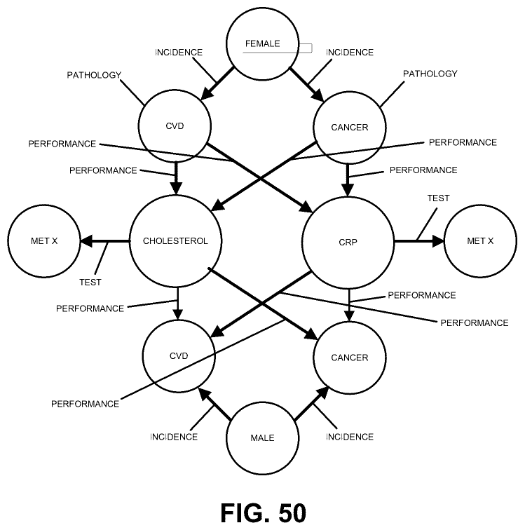

Furthermore, a demographic may have a pathology, and pathology may have a biomarker. FIG. 49 presents a drawing illustrating an example of a graph relating a pathology to a biomarker. Note that this graph may consider that a biomarker is independent of a demographic.

FIG. 50 presents a drawing illustrating another example of a graph relating a pathology to a biomarker. Note that duplication of pathology may allow modification of the performance of a biomarker for a given demographic. Alternatively, a pathology may be defined based at least in part on a range of values of a biomarker.

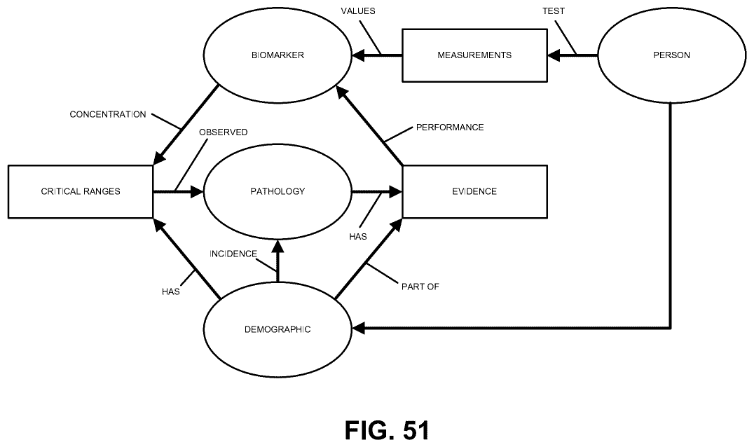

In some embodiments, a combination of a demographic and a pathology may provide evidence. In conjunction with a biomarker (such as a measurement), this may provide relevant information for an individual. FIG. 51 presents a drawing illustrating an example of an evidence relationship. Notably, evidence (e.g., a study or analysis) may support a link between a demographic and a pathology. Evidence may also describe the predictive performance of a biomarker to identify a pathology given a demographic. Moreover, an individual may have a measurement of one or more biomarkers that have critical ranges of values for pathology. Note that there may be multiple types of evidence for a biomarker given a pathology and a demographic. Evidence supporting a link between multiple biomarkers and a pathology may include biomarker mutual information (e.g., partial correlation). Mutual information may be used to calculate compound predictive sensitivity and specificity metrics of biomarkers for a pathology. Furthermore, an individual may have a demographic that has a high incidence of a pathology for which there are optimal biomarkers.

While the preceding discussion illustrated the data-driven physical with one or more operations that are performed based at least in part on expert input (such as decision thresholds), in other embodiments any of the operations in these techniques may be based at least in part on statistical analysis. For example, an operation may be performed (or not) using a statistically determined threshold in a population or group of individuals of 400. Moreover, while the preceding discussion of user-interface techniques used tracking of doctor or medical-professional selections, more generally tracking of user actions or lack of action may be used. For example, the training data may include: hovering in an instance of a web page or website, lack of a selection in the instance of the web page or website, repeated navigation to an image in the instance of the web page or website, etc. Thus, the user action(s) and/or lack of action(s) in the instance of the web page or website may provide relevance information that is used for training and, therefore, to revise a future instance of the web page or website.

Furthermore, while the preceding discussion illustrated the data-driving physical and user-interface techniques with MR images, more generally these techniques may be used with arbitrary measurements and, thus, are not restricted to images. For example, the data in the techniques may include: wearable device data (such as one or more vital signs, accelerometer data, location data, etc.), anatomical data, genetics data, medical record data, etc. Moreover, the data in the techniques may include invasive data (in addition to or instead of noninvasive data). For example, the data may include: biopsy data, medical notes (such as subjective observations from a physician or healthcare provider), etc. Thus, the computation techniques may be used with an arbitrary type of measurements (and are not restricted to images and/or biomarkers).

We now further describe an electronic device that performs at least some of the operations in, e.g., the embodiments of the computing techniques, computation techniques and/or the analysis techniques. FIG. 52 presents a block diagram illustrating an electronic device 5200 in system 100 (FIG. 1), such as computer system 116 (FIG. 1) or another of the computer-controlled components in system 100, such as source 110 or measurement device 114 (FIG. 1). This electronic device includes a processing subsystem 5210, memory subsystem 5212, and networking subsystem 5214. Processing subsystem 5210 may include one or more devices configured to perform computational operations and to control components in system 100 (FIG. 1). For example, processing subsystem 5210 may include one or more microprocessors or central processing units (CPUs), one or more graphics processing units (GPUs), application-specific integrated circuits (ASICs), microcontrollers, programmable-logic devices (such as a field programmable logic array or FPGA), and/or one or more digital signal processors (DSPs).

Memory subsystem 5212 may include one or more devices for storing data and/or instructions for processing subsystem 5210 and networking subsystem 5214. For example, memory subsystem 5212 may include dynamic random access memory (DRAM), static random access memory (SRAM), and/or other types of memory. In some embodiments, instructions for processing subsystem 5210 in memory subsystem 5212 include one or more program modules or sets of instructions (such as program instructions 5224), which may be executed in an operating environment (such as operating system 5222) by processing subsystem 5210. Note that the one or more computer programs may constitute a computer-program mechanism or a program module (i.e., software). Moreover, instructions in the various modules in memory subsystem 5212 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 5210.

In addition, memory subsystem 5212 may include mechanisms for controlling access to the memory. In some embodiments, memory subsystem 5212 includes a memory hierarchy that comprises one or more caches coupled to a memory in electronic device 5200. In some of these embodiments, one or more of the caches is located in processing subsystem 5210.

In some embodiments, memory subsystem 5212 is coupled to one or more high-capacity mass-storage devices (not shown). For example, memory subsystem 5212 may be coupled to a magnetic or optical drive, a solid-state drive, or another type of mass-storage device. In these embodiments, memory subsystem 5212 may be used by electronic device 5200 as fast-access storage for often-used data, while the mass-storage device is used to store less frequently used data.

In some embodiments, memory subsystem 5212 includes a remotely located archive device. This archive device can be a high-capacity network attached mass-storage device, such as: network attached storage (NAS), an external hard drive, a storage server, a cluster of servers, a cloud-storage provider, a cloud-computing provider, a magnetic-tape backup system, a medical records archive service, and/or another type of archive device. Moreover, processing subsystem 5210 may interact with the archive device via an application programming interface to store and/or access information from the archive device. Note that memory subsystem 5212 and/or electronic device 5200 may be compliant with the Health Insurance Portability and Accountability Act.

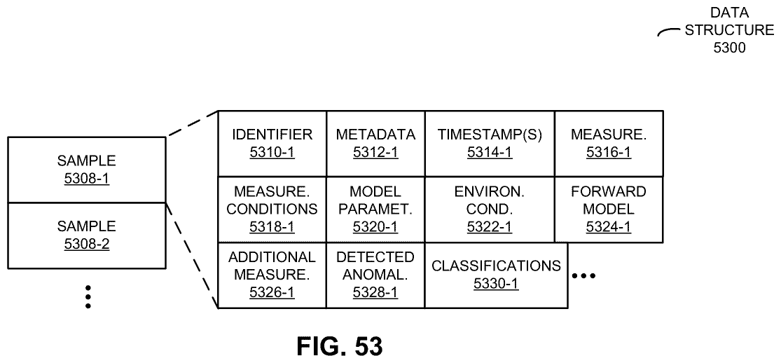

An example of the data stored (locally and/or remotely) in memory subsystem 5212 is shown in FIG. 53, which presents a drawing illustrating an example of a data structure 5300 that is used by electronic device 5200 (FIG. 52). This data structure may include: an identifier 5310-1 of sample 5308-1 (such as an individual), metadata 5312 (such as age, gender, biopsy results and diagnosis if one has already been made, other sample information, demographic information, family history, etc.), timestamps 5314 when data was acquired, received measurements 5316 (such as MR signals and, more generally, raw data), excitation and measurement conditions 5318 (such as an external magnetic field, an optional gradient, an RF pulse sequence, an MR apparatus, a location, machine-specific characteristics such as magnetic-field inhomogeneity, RF noise and one or more other system imperfections, signal-processing techniques, registration information, synchronization information such between measurements and a heartbeat or breathing pattern of an individual, etc.), and/or determined model parameters 5320 (including voxel sizes, speed, resonant frequency or a type of nuclei, T₁ and T₂ relaxation times, segmentation information, classification information, etc.), environmental conditions 5322 (such as the temperature, humidity and/or barometric pressure in the room or the chamber in which sample 5308-1 was measured), forward model 5324, one or more additional measurements 5326 of physical properties of sample 5308-1 (such as weight, dimensions, images, etc.), optional detected anomalies 5328 (which may include particular voxel(es) associated with the one or more of detected anomalies 5328), and/or optional classifications 5330 of the one or more detected anomalies 5328. Note that data structure 5300 may include multiple entries for different measurements.

In one embodiment, data in data structure 5300 is encrypted using a block-chain or a similar cryptographic hash technique to detect unauthorized modification or corruption of records. Moreover, the data can be anonymized prior to storage so that the identity of an individual associated with a sample is anonymous unless the individual gives permission or authorization to access or release the individual's identity.

Referring back to FIG. 52, networking subsystem 5214 may include one or more devices configured to couple to and communicate on a wired, optical and/or wireless network (i.e., to perform network operations and, more generally, communication), including: control logic 5216, an interface circuit 5218, one or more antennas 5220 and/or input/output (I/O) port 5228. (While FIG. 52 includes one or more antennas 5220, in some embodiments electronic device 5200 includes one or more nodes 5208, e.g., a pad or connector, which can be coupled to one or more antennas 5220. Thus, electronic device 5200 may or may not include one or more antennas 5220.) For example, networking subsystem 5214 can include a Bluetooth networking system (which can include Bluetooth Low Energy, BLE or Bluetooth LE), a cellular networking system (e.g., a 3G/4G/5G network such as UMTS, LTE, etc.), a universal serial bus (USB) networking system, a networking system based on the standards described in IEEE 802.11 (e.g., a Wi-Fi networking system), an Ethernet networking system, and/or another networking system.

Moreover, networking subsystem 5214 may include processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for network subsystem 5214. Moreover, in some embodiments a ‘network’ between components in system 100 (FIG. 1) does not yet exist. Therefore, electronic device 5200 may use the mechanisms in networking subsystem 5214 for performing simple wireless communication between the components, e.g., transmitting advertising or beacon frames and/or scanning for advertising frames transmitted by other components.

Within electronic device 5200, processing subsystem 5210, memory subsystem 5212, networking subsystem 5214 may be coupled using one or more interconnects, such as bus 5226. These interconnects may include an electrical, optical, and/or electro-optical connection that the subsystems can use to communicate commands and data among one another. Although only one bus 5226 is shown for clarity, different embodiments can include a different number or configuration of electrical, optical, and/or electro-optical connections among the subsystems.

Electronic device 5200 may be (or can be) included in a wide variety of electronic devices. For example, electronic device 5200 may be included in: a tablet computer, a smartphone, a smartwatch, a portable computing device, a wearable device, test equipment, a digital signal processor, a cluster of computing devices, a laptop computer, a desktop computer, a server, a subnotebook/netbook and/or another computing device.

Although specific components are used to describe electronic device 5200, in alternative embodiments, different components and/or subsystems may be present in electronic device 5200. For example, electronic device 5200 may include one or more additional processing subsystems, memory subsystems, and/or networking subsystems. Additionally, one or more of the subsystems may not be present in electronic device 5200. Moreover, in some embodiments, electronic device 5200 may include one or more additional subsystems that are not shown in FIG. 52.

Although separate subsystems are shown in FIG. 52, in some embodiments, some or all of a given subsystem or component can be integrated into one or more of the other subsystems or components in electronic device 5200. For example, in some embodiments program instructions 5224 are included in operating system 5222. In some embodiments, a component in a given subsystem is included in a different subsystem. Furthermore, in some embodiments electronic device 5200 is located at a single geographic location or is distributed over multiple different geographic locations.

Moreover, the circuits and components in electronic device 5200 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit may implement some or all of the functionality of networking subsystem 5214 (such as a radio) and, more generally, some or all of the functionality of electronic device 5200. Moreover, the integrated circuit may include hardware and/or software mechanisms that are used for transmitting wireless signals from electronic device 5200 and receiving signals at electronic device 5200 from other components in system 100 (FIG. 1) and/or from electronic devices outside of system 100 (FIG. 1). Aside from the mechanisms herein described, radios are generally known in the art and hence are not described in detail. In general, networking subsystem 5214 and/or the integrated circuit can include any number of radios. Note that the radios in multiple-radio embodiments function in a similar way to the radios described in single-radio embodiments.

While some of the operations in the preceding embodiments were implemented in hardware or software, in general the operations in the preceding embodiments can be implemented in a wide variety of configurations and architectures. Therefore, some or all of the operations in the preceding embodiments may be performed in hardware and/or in software.

In addition, in some of the preceding embodiments there are fewer components, more components, a position of a component is changed and/or two or more components are combined.

While the preceding discussion illustrated the use of the computation techniques to solve a vector wave equation, in other embodiments the computation techniques may be used to solve a scalar equation. For example, an acoustic wave equation may be solved in an arbitrary inhomogeneous media based on ultrasound measurements using a forward model. (Thus, in some embodiments the excitation may be mechanical.) Note that the acoustic coupling in ultrasound measurements can dependent on the operator (i.e., the ultrasound measurements may be pressure dependent). Nonetheless, a similar approach may be used to: improve ultrasound imaging, determine 3D structure, facilitate improved presentation, etc.

In the preceding description, we refer to ‘some embodiments.’ Note that ‘some embodiments’ describes a subset of all of the possible embodiments, but does not always specify the same subset of embodiments. Moreover, note that numerical values in the preceding embodiments are illustrative examples of some embodiments. In other embodiments of the computation techniques and/or the analysis techniques, different numerical values may be used.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. A computer system, comprising:
a processor configured to execute program instructions; and
memory storing the program instructions, wherein, when executed by the processor, the program instructions cause the computer system to perform operations comprising:
receiving information specifying selected content;
dynamically determining related content based at least in part on one or more prior expert selections that are associated with the selected content, one or more predefined or predetermined relationships with the selected content, or both; and
providing the selected content and the determined related content.

2. The computer system of claim 1, wherein the selected content comprises an image associated with a non-invasive characterization technique.

3. The computer system of claim 2, wherein the non-invasive characterization technique comprises: magnetic resonance imaging (MRI), computed tomography, x-ray imaging or ultrasound.

4. The computer system of claim 2, wherein the image corresponds to a portion of a human body.

5. The computer system of claim 2, wherein the related content comprises a measurement associated with a different measurement technique than the non-invasive characterization technique.

6. The computer system of claim 1, wherein the selected content comprises or is associated with a biomarker, and the one or more predefined or predetermined relationships are between the biomarker and one or more additional biomarkers.

7. The computer system of claim 1, wherein the one or more predefined or predetermined relationships are based at least in part on: genetics information, chemistry, a behavior, an environmental factor, a disease, a comorbidity or a biological pathway.

8. The computer system of claim 1, wherein the one or more predefined or predetermined relationships may be based at least in part on medical literature.

9. The computer system of claim 1, wherein the content is associated with a patient, and the one or more prior expert selections are made by a physician and are associated with one or more different patients from the patient.

10. The computer system of claim 9, wherein the patient and the one or more different patients share one or more characteristics or attributes.

11. The computer system of claim 9, wherein the patient and the one or more different patients have a disease, a risk of having the disease, or members of a demographic group or a common population.

12. The computer system of claim 1, wherein providing the selected content and the determined related content comprises displaying the selected content and the determined related content on a display.

13. The computer system of claim 1, wherein providing the determined related content comprises providing a user interface with user-interface features corresponding to the determined related content.

14. A non-transitory computer-readable storage medium for use in conjunction with a computer system, the computer-readable storage medium configured to store a program module that, when executed by the computer system, causes the computer system to perform operations comprising:
receiving information specifying selected content;
dynamically determining related content based at least in part on one or more prior expert selections that are associated with the selected content, one or more predefined or predetermined relationships with the selected content, or both; and
providing the selected content and the determined related content.

15. The non-transitory computer-readable storage medium of claim 14, wherein the selected content comprises an image associated with a non-invasive characterization technique; and
wherein the related content comprises a measurement associated with a different measurement technique than the non-invasive characterization technique.

16. The non-transitory computer-readable storage medium of claim 14, wherein the content is associated with a patient, and the one or more prior expert selections are made by a physician and are associated with one or more different patients from the patient.

17. A method for dynamically determining related content, comprising:
by a computer system:
receiving information specifying selected content;
dynamically determining the related content based at least in part on one or more prior expert selections that are associated with the selected content, one or more predefined or predetermined relationships with the selected content, or both; and
providing the selected content and the determined related content.

18. The method of claim 17, wherein the selected content comprises an image associated with a non-invasive characterization technique; and
wherein the related content comprises a measurement associated with a different measurement technique than the non-invasive characterization technique.

19. The method of claim 17, wherein the content is associated with a patient, and the one or more prior expert selections are made by a physician and are associated with one or more different patients from the patient.

20. The method of claim 17, wherein providing the selected content and the determined related content comprises displaying the selected content and the determined related content on a display; and
wherein providing the determined related content comprises providing a user interface with user-interface features corresponding to the determined related content.
